(19) United States
(12) Patent Application Publication
(10) Pub. No.: US 2026/0062703 A1
(43) Pub. Date: Mar. 5, 2026

(54) Delivery of RNA Therapeutics Using Circular Prodrug Nucleic Acids

(71) Applicant: ARNAY Sciences, LLC, Shrewsbury, MA (US)

(72) Inventor: Sudhir AGRAWAL, Shrewsbury, MA (US)

(21) Appl. No.: 19/332,120

(22) Filed: Sep. 18, 2025

Related U.S. Application Data
(63) Continuation of application No. PCT/US24/20897, filed on Mar. 21, 2024.
(60) Provisional application No. 63/453,909, filed on Mar. 22, 2023.

(51) Int. Cl. C12N 15/113

(52) U.S. Cl. CPC C12N 15/113; C12N 2310/11; C12N 2310/315; C12N 2310/532

(57) ABSTRACT

The present invention provides oligonucleotides referred to as circular prodrug nucleic acid (“CPN”) as described herein, compositions comprising same, and methods of using same. This design of circular prodrug nucleic acids maintains a circular form until the circularizing domain is cleaved in situ by RNase H or Dicer or other intracellular factors.

### RELATED APPLICATIONS

This application is a continuation of International Application No. PCT/US24/20897, which designated the United States and was filed on Mar. 21, 2024, published in English, which claims the benefit of U.S. Provisional Application No. 63/453,909, filed on Mar. 22, 2023. The entire teachings of the above applications are incorporated herein by reference.

### SEQUENCE LISTING

The sequence listing submitted via EFS, in compliance with 37 CFR § 1.52(e)(5), is incorporated herein by reference. The sequence listing XML file submitted via EFS contains the file 42503003US1SEQLISTING.xml, created on Sep. 18, 2025, which is 323,106 bytes in size.

### BACKGROUND

The processing and translation of targeted RNA can be modulated by multiple mechanisms using antisense oligonucleotides. These include cleaving the targeted RNA by RNase-H, modulating aberrant splicing, processing of targeted RNA and increased translation, inhibiting the translation by steric hindrance etc. Targeted RNA could be mRNA or noncoding RNA. In other mechanisms, the antisense strand of a duplex siRNA could be incorporated in AGO and inhibit translation by the siRNA mechanism. In other mechanisms, like adenosine deaminase acting on RNA (ADAR) based or CRISPR-based models, antisense could edit RNA or DNA and thereby modulate translation and processing.

Over the years it has been learned that antisense hybridization and affinity influence selectivity for the targeted RNA. In addition, to use antisense nucleic acids as drugs, nuclease stability is important which has been provided by the modification of internucleotide linkages, for example, phosphorothioate.

It was postulated that nuclease stability is key for survival in intracellular compartments and potency and since the degradation of antisense was shown to be from the 3′-end, the focus was to modify the 3′-end to slow down degradation. These designs include capping on the 3′-end, and a hairpin loop on the 3′-end, creating oligos having secondary structures comprising 3′-3′ linkages or attaching two (2) antisense oligonucleotides at their 3′ ends. These type of antisense showed increased nuclease stability but antisense potency was not improved. Unfortunately, these modifications also increased inflammatory responses thereby limiting the therapeutic index.

Both DNA and RNA and 2′-substituted RNA containing phosphorothioate have been studied as antisense agents and provide different characteristics. DNA phosphorothioate antisense, when hybridized to RNA, activates RNase H, whereas RNA or 2′-substituted RNA antisense binds to RNA with higher affinity and does not activate RNase H. To further improve antisense characteristics, a mixture of these two modifications has been employed in antisense, generally referred to as hybrid or gapmer antisense. In most studied gapmer antisense, modified RNA segment is placed on both the 3′- and 5′-end whereas DNA is placed in the middle. Gapmer antisense is the most widely studied antisense and drugs employing this chemistry are approved and are in clinical development.

One of the side-effects of both DNA and RNA phosphorothioate is due to the interaction with proteins and more specifically with the family of Pattern Recognition Receptors (PRRs). These interactions result in induction of an immune cascade thereby causing an off-target mechanism of action and related safety signals. Detailed structure-activity relationship studies have shown that the accessibility of the 5′-end of DNA and RNA phosphorothioate antisense is required for immune activation. DNA and RNA phosphorothioate containing two 5′-ends have shown increased immuno-stimulatory activity. In contrast, it has been previously shown that DNA and RNA phosphorothioate which contain two 3′-ends (and lack a free 5′-end) show minimal inflammatory responses.

In continuing efforts to improve the properties of DNA and modified RNA phosphorothioate antisense as therapeutic agents, structural changes in oligonucleotides were considered. For example, in earlier studies, self-stabilized oligonucleotides were reported wherein oligodeoxynucleotide phosphorothioates (“PS-oligonucleotide”) containing a hairpin loop region at the 3′-end provided increased in vivo nuclease stability and limited biological activity. To date focus has been to improve stability of antisense by modifying 3′-end by various modifications including in gapmer antisense.

Despite the advances that have been made, there is still a desire to develop antisense oligonucleotides and RNA therapeutics having improved properties for use as therapeutic agents and in diagnostic applications.

### SUMMARY OF THE INVENTION

The present invention provides a structural class of oligonucleotides referred to herein as “circular prodrug nucleic acid” (CPN) or, equivalently, “circular prodrug oligos” (CPO). A CPN comprises a “functional domain” and a “circularizing domain”.

The “functional domain” comprises an oligonucleotide that provides a function to the CPN.

In embodiments, the “circularizing domain” comprises a first nucleic acid molecule and a second nucleic acid molecule, wherein the 5′ end of the oligonucleotide of the functional domain is linked (directly or through a linker segment) to the first nucleic acid molecule and the 3′ end of the oligonucleotide of the functional domain is linked to the second nucleic acid molecule; wherein the first nucleic acid molecule and the second nucleic acid molecule are independently 6 to 30 nucleotides in length, wherein the nucleotides of the first nucleic acid molecule and the second nucleic acid molecule are independently selected from RNA or DNA or a combination of RNA and DNA, and wherein the first nucleic acid molecule and the second nucleic acid molecule are complementary to each other and of opposite polarity to each other and hybridize to form a double-stranded section. (e.g., FIG. 1)

In embodiments, the “circularizing domain” comprises a first nucleic acid molecule and a second nucleic acid molecule, wherein the 5′ end of the oligonucleotide of the functional domain is linked (directly or through a linker segment) to the first nucleic acid molecule and the 3′ end of the oligonucleotide of the functional domain is linked to the second nucleic acid molecule; wherein the first nucleic acid molecule and the second nucleic acid molecule are independently 6 to 30 nucleotides in length, wherein the nucleotides of the first nucleic acid molecule and the second nucleic acid molecule are independently selected from RNA or DNA or a combination of RNA and DNA, provided that the first nucleic acid molecule and the second nucleic acid molecule are not both DNA, and wherein the first nucleic acid molecule and the second nucleic acid molecule are complementary to each other and of opposite polarity to each other and hybridize to form a double-stranded section. (e.g., FIG. 1)

The oligonucleotide of the functional domain and the first and second nucleic acid molecules of the circularizing domain are further described herein.

CPNs of the invention adopts an intramolecular circular structure due to the complementarity between and the first nucleic acid molecule and second nucleic acid molecule of the circularizing domain, which form an intramolecular duplex. This intramolecular duplex formation changes the functional domain's shape and accessibility to the 5′ and 3′ ends of oligonucleotide of the functional domain. This structure combines key attributes to create optimal nucleic acid-based therapeutics. In the CPN, the nucleotides of the oligonucleotide of the functional domain do not participate in forming the intramolecular duplex.

Improving RNA therapeutics is to improve the delivery of nucleic acids to cytoplasm and nucleus in a stable form can improve the usefulness of RNA therapeutics. The 3′- and 5′-ends of nucleic acids bind to pattern recognition receptors and exonucleases thereby impact RNA therapeutics' availability. Therefore, delivery of RNA therapeutic agents to the appropriate compartment by making the ends inaccessible to these factors would help improve RNA therapeutics. The conditional release of RNA therapeutics intracellularly by factors present in nature would permit the agent to be available in the desired compartment.

In gene and RNA expression modulation, this structure masks the 5′-end of the oligonucleotide functional domain thereby reducing the interaction with PRRs and permitting endosomal escape. The structure of CPN also masks 3′-end to provide nuclease stability. Once in the cytoplasm or nucleus, the double-stranded section formed by the first nucleic acid molecule and the second nucleic acid molecule will be cleaved by RNase H, Dicer, restriction enzymes, or other intracellular factors and the circular structure will open, thereby presenting the oligonucleotide of the functional domain and allowing it to perform its function. This process of cleaving the circularizing domain of the CPN to present the oligonucleotide of the functional domain is referred to herein as “in situ activation”.

In embodiments, once cleaved, the first nucleic acid molecule and second nucleic acid molecule of the circularizing domain cannot hybridize, thereby preventing the re-circularization of the oligonucleotide of the functional domain.

When the CPN is in the circular form, it may exhibit fewer of the polyanionic-related side effects (e.g., complement activation and prolongation of partial thromboplastin time) known to occur with PS-oligonucleotides, because there are fewer exposed phosphorothioate linkages or reduced number of PS linkages. Also, CPNs in circular form would have reduced protein binding, reduced off-target interactions with non-targeted RNAs, and increased nuclease stability and endosomal escape.

CPNs according to the invention can be made using standard techniques for synthesis of the constituent oligonucleotides and are useful for all purposes for which the functional oligonucleotide and nucleic acid is useful.

The foregoing merely summarizes certain aspects of the invention and is not intended, nor should it be construed, as limiting the invention in any manner. All patents, patent applications, and other publications recited in this specification are hereby incorporated by reference in their entirety.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1A through FIG. 1H depicts various embodiments of the circular prodrug oligonucleotide (100) according to the invention. The solid line (101) represents the functional domain. The dashed lines (102) represent a first nucleic acid molecule of the circularizing domain. The hashed lines (103) represent the second nucleic acid molecule of the circularizing domain. L represents the linking of the functional domain and circularizing domains directly or via a linker. the circular prodrug oligonucleotide maintains a circular form until the double-stranded region formed by the first nucleic acid molecule and the second nucleic acid molecule is cleaved in situ by RNase H or Dicer, restriction enzymes, or other intracellular factors, releasing the functional domain. An optional linker, L, can be present in either, or both of, the first or second nucleic acid molecules of the circularizing domain as long as the linker does not interfere with the ability of the first or second nucleic acid molecules to hybridize to each or and to be cleaved in situ.

FIG. 2A through FIG. 2J depicts a comparison of antisense oligonucleotides in circularized pro-drug formats targeting apolipoprotein C-III (APOC3). Data is plotted as percent of control cells (cells reverse-transfected with vehicle alone). Gapmer: 5′-GCTTCTTGTCCAGCTTTATT-3′ (SEQ ID NO: 33). Gapmer non-target control: 5′-CCAAATCTTATAATAACTAC-3′ (SEQ ID NO 36).

FIG. 3A through FIG. 3D depicts a comparison of antisense oligonucleotides in gapmer format with circularized pro-drug formats targeting microtubule-associated protein tau (MAPT). Data is plotted as percent of control cells (cells reverse-transfected with vehicle alone). Gapmer control: 5′-CCGTTTTCTTACCACCCT-3′ (SEQ ID NO: 37).

### DETAILED DESCRIPTION

It is to be understood that both the foregoing general description and the following detailed description are exemplary and explanatory only and are not restrictive.

Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated-by-reference for the portions of the document discussed herein, as well as in their entirety.

Features may be described herein as part of the same or separate aspects or embodiments of the present invention for the purpose of clarity and a concise description. It will be appreciated by the skilled person that the scope of the invention may include embodiments having combinations of all or some of the features described herein as part of the same or separate embodiments.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges and increments and/or portions therein, as well as individual numerical values within that range. For example, description of a range such as from 6 to 30 should be considered to include subranges such as from 6 to 27, from 6 to 25, from 6 to 15, from 8 to 20, from 8 to 15, from 10 to 15, etc., as well as individual numbers with that range, for example, 6, 7, 8, 9, 10, 11, 12, 13, 14, 15, 16, 17, 18, 19, 20, 21, 22, 23, 24, 25, 26, 27, 28, 29, and 30 and further, as applicable, any portions or increments between or of a number, for example, tenths, hundredths, etc. This applies regardless of the breadth of the range.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, including ±5%, ±1%, and ±0.1% from the specified value, as such variations are appropriate to perform the disclosed methods.

The present invention also provides oligonucleotides referred to as circular prodrug nucleic acids (“CPNs”). A CPN comprises a “functional domain” and a “circularizing domain”.

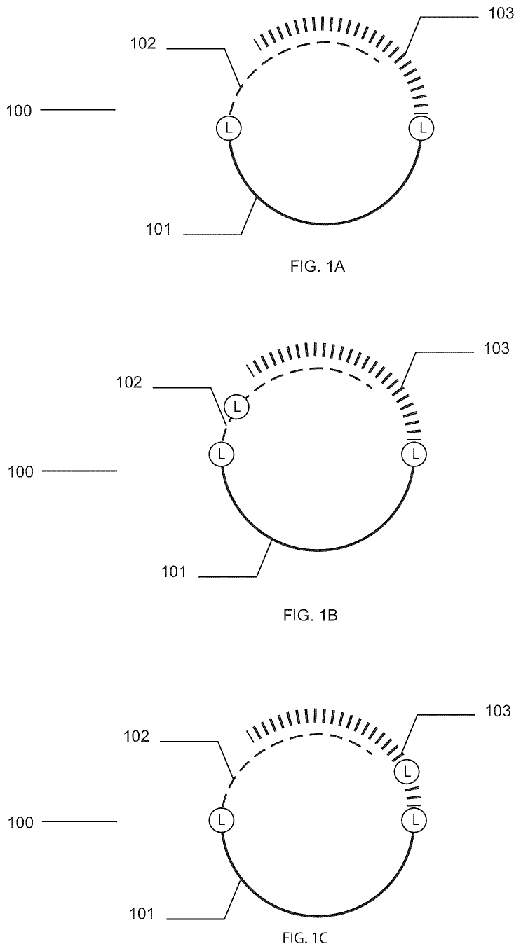

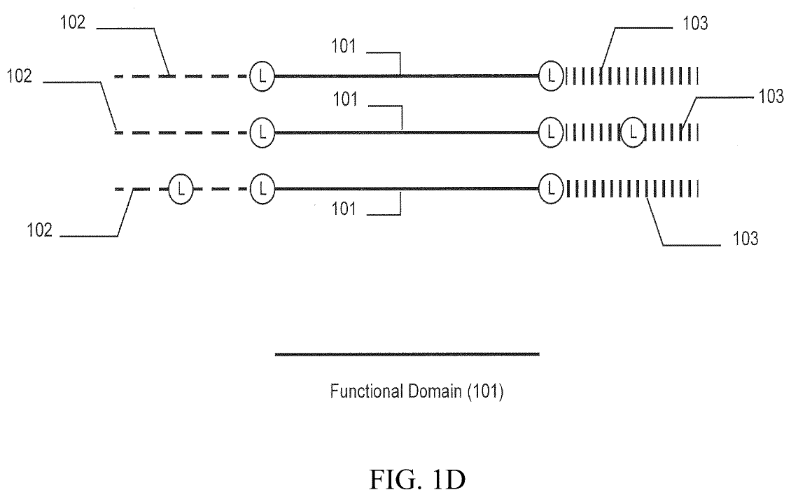

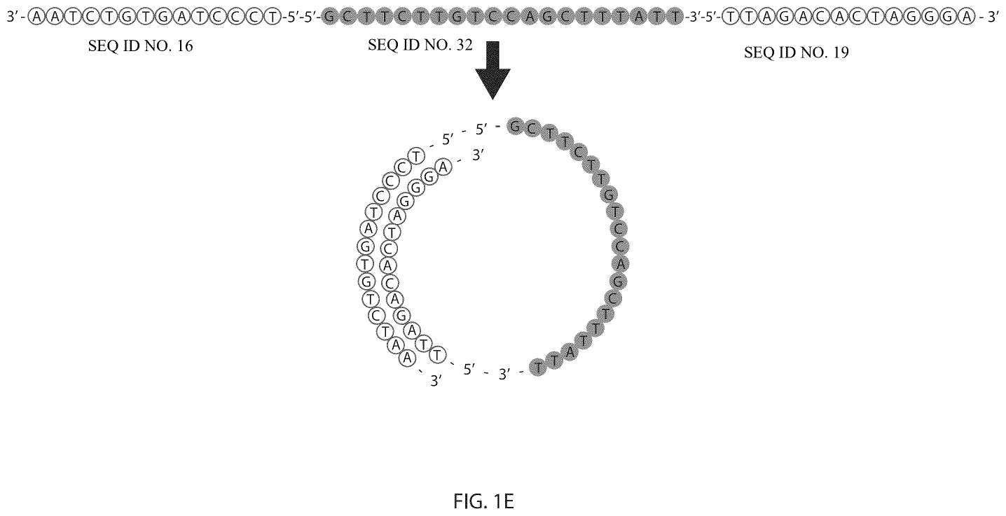

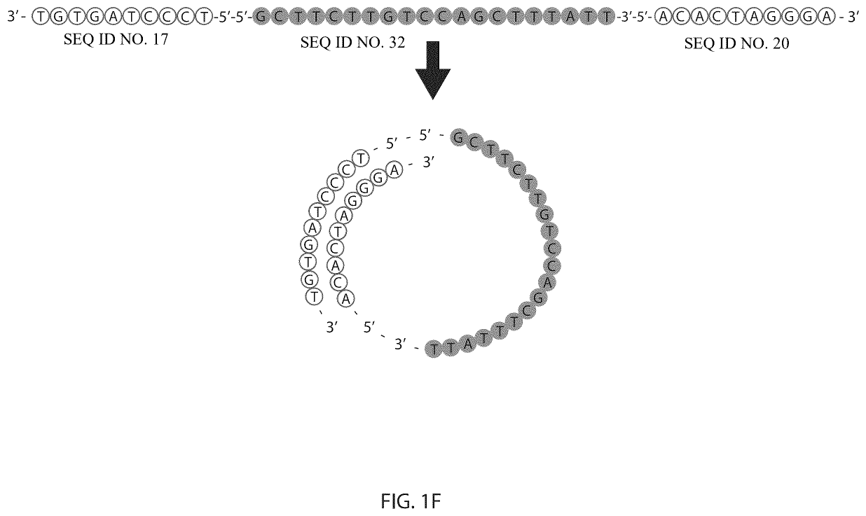

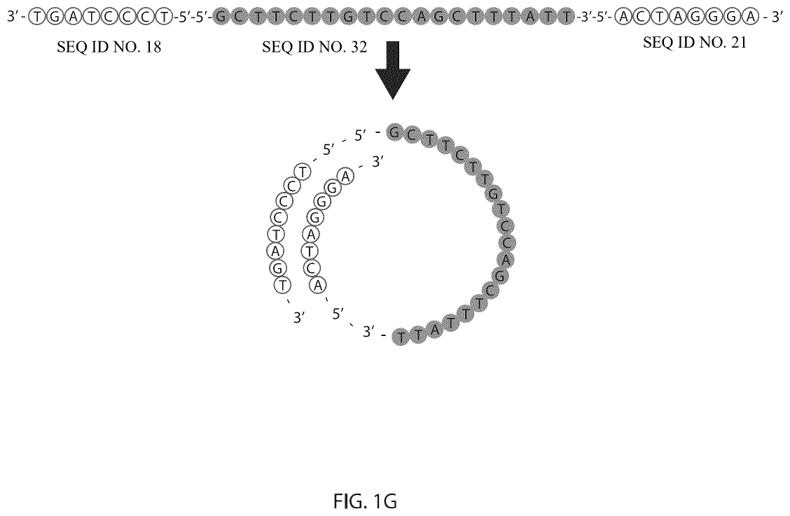

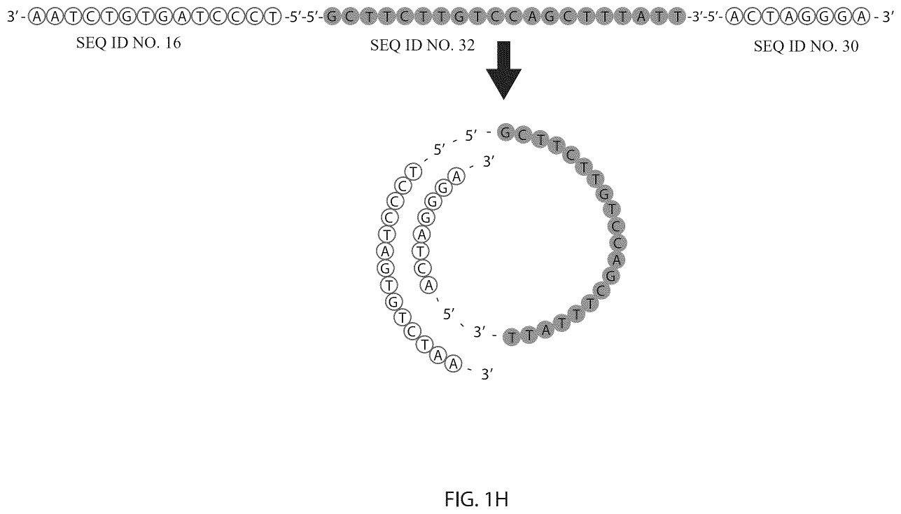

The “functional domain” comprises an oligonucleotide that provides a function to the CPN. The circularizing domain comprises a first nucleic acid molecule and a second nucleic acid molecule, wherein the 5′ end of the oligonucleotide of the functional domain is linked, directly or through a linker segment, to the first nucleic acid molecule and the 3′ end of the oligonucleotide of the functional domain is linked to the second nucleic acid molecule; wherein the first nucleic acid molecule and the second nucleic acid molecule are independently 6 to 30 nucleotides in length, wherein the nucleotides of the first nucleic acid molecule and the second nucleic acid molecule are independently selected from RNA or DNA or a combination of RNA and DNA, and wherein the first nucleic acid molecule and the second nucleic acid molecule are complementary to and of opposite polarity to each other and hybridize to form a double-stranded section (e.g., FIG. 1A-FIG. 1C). In embodiments, the first nucleic acid molecule and the second nucleic acid molecule are not both DNA.

In embodiments, the CPN of the invention comprises a structure according to Formula I or Formula II:

Formula I
3′-YₙY₆Y₅Y₄Y₂Y₂Y₁-5′-5′-functional domain oligonucleotide-3′-5′-X₁X₂X₃X₄X₅X₆Xₘ-3′
Formula II
5′-Y₁Y₂Y₃Y₄Y₅Y₆Yₙ-3′-5′-functional domain oligonucleotide-3′-3′-XₘX₆X₅X₄X₃X₂X₁-5′

wherein
Y₁-Yₙ is a first nucleic acid molecule as defined herein;
X₁-Xₘ is a second nucleic acid as defined herein;
n is 0-44; and
m is 0-44. The functional domain oligonucleotide is any oligonucleotide as defined herein.

In embodiments, the CPN of the invention comprises a structure according to Formula III, IV, V, VI, or VII:

Formula III
3′-YₙY₆Y₅Y₄Y₂Y₂Y₁-5′-L-5′-functional domain oligonucleotide-3′-5′-X₁X₂X₃X₄X₅X₆Xₘ-3′
Formula IV
5′-Y₁Y₂Y₃Y₄Y₅Y₆Yₙ-3′-L-5′-functional domain oligonucleotide-3′-3′-XₘX₆X₅X₄X₃X₂X₁-5′
Formula V
3′-YₙY₆Y₅Y₄Y₂Y₂Y₁-5′-5′-functional domain oligonucleotide-3′-L-5′-X₁X₂X₃X₄X₅X₆Xₘ-3′
Formula VI
5′-Y₁Y₂Y₃Y₄Y₅Y₆Yₙ-3′-5′-functional domain oligonucleotide-3′-L-3′-XₘX₆X₅X₄X₃X₂X₁-5′
Formula VII
5′-Y₁Y₂Y₃Y₄Y₅Y₆Yₙ-3′-L-5′-functional domain oligonucleotide-3′-L-3′-XₘX₆X₅X₄X₃X₂X₁-5′

wherein
Y₁-Yₙ is a first nucleic acid molecule as defined herein;
X₁-Xₘ is a second nucleic acid as defined herein;
L is a linker as defined herein;
n is 0-44; and
m is 0-44. The functional domain oligonucleotide is any oligonucleotide as defined herein.

In embodiments, the first nucleic acid molecule is linked to the 5′-end of the functional domain with a 5′-5′ linkage and the second nucleic acid molecule is linked to the 3′ of the oligonucleotide with a 3′-5′ linkage. In embodiments, the first nucleic acid molecule is linked to the 5′-end of the functional domain with a 5′-3′ linkage, and the second nucleic acid molecule is linked to the 3′ of the oligonucleotide with a 3′-3′ linkage.

In either configuration, the first nucleic acid molecule and the second nucleic acid molecule of the circularizing domain are complementary to each other and of opposite polarity to each other allowing the first nucleic acid molecule to hybridize to the second nucleic acid molecule, thereby forming a circular structure (e.g., FIG. 1A-FIG. 1C). This design of circular prodrug nucleic acid maintains a circular form until the double stranded region is cleaved by intracellular factors, such as RNase H and Dicer and others. This structure allows for increased stability, specificity and release of functional domain in the appropriate compartment.

The oligonucleotide of the functional domain provides a desired function to the CPN. For example, for gene expression modulation, the oligonucleotide of the functional domain is complementary to a targeted RNA. As used herein, the terms “oligonucleotide of the functional domain” or the “functional domain” are used interchangeably.

CPNs of the invention adopts an intramolecular circular structure due to the complementarity between and the first nucleic acid molecule and second nucleic acid molecule of the circularizing domain, which form an intramolecular duplex. The nucleotides of the oligonucleotide of the functional domain do not participate in the formation of the intramolecular duplex.

As the skilled artisan will recognize, despite the requirement that the nucleotides of the oligonucleotide of the functional domain do not participate in the formation of the intramolecular duplex in the CPN of the instant invention, one or more nucleotides at the 5′ and/or 3′ end of oligonucleotide of the functional domain could be designed to participate in the duplex formation leading to the circular shape so long as that, upon cleave of the duplex of the CPN, and thereby the linearization of the oligonucleotide compound, the oligonucleotide of the functional domain can still perform its role.

The nucleotides of the first nucleic acid molecule and second nucleic acid molecule of the circularizing domain are not part of the oligonucleotide of the functional domain and, therefore, do not provide the desired function to the CPN.

In embodiments, the oligonucleotide of the functional domain is DNA or RNA or combinations thereof.

In embodiments, the first nucleic acid molecule of the circularizing domain is DNA and the second nucleic acid molecule is RNA. In embodiments, the first nucleic acid molecule of the circularizing domain is RNA and the second nucleic acid molecule is DNA. In embodiments, the first nucleic acid molecule of the circularizing domain is RNA and the second nucleic acid molecule is also RNA. In embodiments, the first nucleic acid molecule of the circularizing domain is DNA and the second nucleic acid molecule is also DNA. In embodiments, the first nucleic acid molecule and the second nucleic acid molecule of the circularizing domain are not both DNA.

In embodiments, the nucleotides of the first nucleic acid molecule and/or the second nucleic acid molecule of the circularizing domain are a combination of RNA and DNA.

In embodiments, the oligonucleotide of the functional domain is unmodified.

In embodiments, the first nucleic acid molecule and the second nucleic acid molecule of the circularizing domain are unmodified.

By “unmodified” it is intended that the nucleotides of the of oligonucleotide of the functional domain and/or the first nucleic acid molecule and the second nucleic acid molecule comprise naturally occurring nucleobases, sugars, and internucleotide backbones.

In embodiments, at least one nucleotide of the oligonucleotide of the functional domain is modified. In embodiments, two or more nucleotides of the oligonucleotide of the functional domain are modified. In embodiments, at least half of the nucleotides of the oligonucleotide of the functional domain are modified. In embodiments, all of the nucleotides of the oligonucleotide of the functional domain are modified.

In embodiments, the nucleotides of the oligonucleotide of the functional domain comprises a modification of the inter-nucleotide linkage, sugar, heterocyclic base, or a combination thereof. These modifications could also be appropriately placed at specific positions within the oligonucleotide of the functional domain. Other chemistries and modification are known in the field of oligonucleotides that can be readily used in accordance with the disclosure and are encompassed within the term ‘modified’ as used in the context of an oligonucleotide herein.

Except as otherwise described herein, the functional domain comprises an oligonucleotide between 15 and 500 nucleotides in length. In embodiments, the oligonucleotide of the functional domain is 17 and 300 nucleotides in length. In embodiments, the oligonucleotide of the functional domain is 17 and 200 nucleotides in length. In embodiments, the oligonucleotide of the functional domain is 17 and 100 nucleotides in length. In embodiments, the oligonucleotide of the functional domain is 17 and 50 nucleotides in length. In embodiments, the oligonucleotide of the functional domain is 17 and 25 nucleotides in length.

In embodiments, the oligonucleotide of the functional domain is 50 and 250 nucleotides in length. In embodiments, the oligonucleotide of the functional domain is 50 and 150 nucleotides in length.

In embodiments, the functional domain comprises an oligonucleotide between 15 and 50 nucleotides in length. In embodiments, the functional domain comprises an oligonucleotide between 17 and 40 nucleotides in length. In embodiments, the functional domain comprises an oligonucleotide between 17 and 25 nucleotides in length. In embodiments, the oligonucleotide of the functional domain is 17 and 22 nucleotides in length.

In embodiments, the oligonucleotide of the functional domain is 17, 18, 19, 20, 21, 22, 23, 24, 25, 26, 27, 28, 29, 30, 31, 32, 33, 34, 35, 36, 37, 38, 39, or 40 nucleotides in length. In embodiments, the functional domain is 17 nucleotides in length. In embodiments, the functional domain is 18 nucleotides in length. In embodiments, the functional domain is 19 nucleotides in length. In embodiments, the functional domain is 20 nucleotides in length. In embodiments, the functional domain is 21 nucleotides in length. In embodiments, the functional domain is 22 nucleotides in length. In embodiments, the functional domain is 23 nucleotides in length. In embodiments, the functional domain is 24 nucleotides in length. In embodiments, the functional domain is 25 nucleotides in length. In embodiments, the functional domain is 26 nucleotides in length. In embodiments, the functional domain is 27 nucleotides in length. In embodiments, the functional domain is 28 nucleotides in length. In embodiments, the functional domain is 29 nucleotides in length. In embodiments, the functional domain is 30 nucleotides in length. In embodiments, the functional domain is 31 nucleotides in length. In embodiments, the functional domain is 32 nucleotides in length. In embodiments, the functional domain is 33 nucleotides in length. In embodiments, the functional domain is 34 nucleotides in length. In embodiments, the functional domain is 35 nucleotides in length. In embodiments, the functional domain is 36 nucleotides in length. In embodiments, the functional domain is 37 nucleotides in length. In embodiments, the functional domain is 38 nucleotides in length. In embodiments, the functional domain is 39 nucleotides in length. In embodiments, the functional domain is 40 nucleotides in length.

In embodiments, the functional domain includes, but is not limited to, an oligonucleotide selected from an antisense oligonucleotide, a microRNA (miRNA), a siRNA, a piRNA, a hnRNA, a ncRNA, a snRNA, a miRNA mimic, a sgRNA, an esiRNA, a shRNA, a lncRNA, a mRNA, a CRISPR-based system, an adenosine deaminase acting on RNA (ADAR) system, or a splicing oligonucleotide.

In embodiments, the oligonucleotide of the functional domain could also be an aptamer.

In embodiments, the functional domain could also be an adeno-associated virus (AAV).

In embodiments, the functional domain includes, but is not limited to, an oligonucleotide selected from an immunostimulatory oligonucleotide or an immune-inhibitory oligonucleotide, also referred to as an immune antagonist oligonucleotide.

The only limitation on the nucleotides and internucleotide linkages of the oligonucleotide of the functional domain is that they do not eliminate the ability of the functional domain to carry out its intended function (e.g., in the case of a functional domain that is an antisense oligonucleotide, to hybridize to and form a duplex with a complementary RNA segment under physiological conditions, which duplex is a substrate for RNase H). Preferred nucleotides and internucleotide linkages are those that will enhance the stability of the CPN to nucleases and other forms of chemical degradation and/or enhance the ability of the functional domain to carry out its intended function.

In embodiments, the internucleotide linkages of the functional domain are phosphorothioate internucleotide linkages, phosphodiester internucleotide linkages or a combination thereof.

In embodiments, the nucleotides of the first nucleic acid molecule and the second nucleic acid molecule of the circularizing domain comprise a modification of the inter-nucleotide linkage, sugar, heterocyclic base, or a combination thereof. These modifications could also be placed at specific positions within the first nucleic acid molecule and the second nucleic acid molecule of the circularizing domain.

As used herein, the terms “the first nucleic acid molecule and the second nucleic acid molecule of the circularizing domain” or the “circularizing domain” are used interchangeably.

In embodiments, the internucleotide linkages of the first nucleic acid molecule and the second nucleic acid molecule of the circularizing domain are phosphorothioate internucleotide linkages, phosphodiester internucleotide linkages or a combination thereof. In embodiments, the internucleotide linkages of the first nucleic acid molecule and the second nucleic acid molecule of the circularizing domain can be modified as known by one skilled in the art provided that the circularizing domain remains a substrate for RNase H or Dicer or other intracellular proteins. In embodiments, the internucleotide linkages of the circularizing domain are phosphodiester internucleotide linkages.

In embodiments, the first nucleic acid molecule and the second nucleic acid molecule each are independently DNA or RNA. In embodiments, the first nucleic acid molecule is DNA and the second nucleic acid molecule is RNA. In embodiments, the first nucleic acid molecule is RNA and the second nucleic acid molecule is DNA. In embodiments, the first nucleic acid molecule is RNA and the second nucleic acid molecule is RNA.

In embodiments, the first nucleic acid molecule is DNA and the second nucleic acid molecule is DNA.

In embodiments, the nucleotides of the first nucleic acid molecule and/or the second nucleic acid molecule are a combination of DNA and RNA. In embodiments, the first nucleic acid molecule is DNA and the second nucleic acid molecule is a combination of RNA and DNA. In embodiments, the first nucleic acid molecule is a combination of RNA and DNA and the second nucleic acid molecule is DNA. In embodiments, the first nucleic acid molecule is RNA and the second nucleic acid molecule is a combination of RNA and DNA. In embodiments, the first nucleic acid molecule is a combination of RNA and DNA and the second nucleic acid molecule is RNA. In embodiments, the first nucleic acid molecule is a combination of RNA and DNA and the second nucleic acid molecule is a combination of RNA and DNA.

In embodiments, the RNA and/or DNA of the first nucleic acid molecule and the second nucleic acid molecule are unmodified. In embodiments, the first nucleic acid molecule and the second nucleic acid molecule each independently comprises an oligonucleotide between 6 and 50 nucleotides in length. In embodiments, the first nucleic acid molecule and the second nucleic acid molecule of the circularizing domain each independently comprises an oligonucleotide between 6 and 25 nucleotides in length. In embodiments, the first nucleic acid molecule and the second nucleic acid molecule independently comprises an oligonucleotide between 6 and 12 nucleotides in length. In embodiments, the first nucleic acid molecule and the second nucleic acid molecule independently comprises an oligonucleotide between 6 and 10 nucleotides in length. In embodiments, the first nucleic acid molecule and the second nucleic acid molecule independently comprises an oligonucleotide between 6 and 8 nucleotides in length.

In embodiments, the first nucleic acid molecule and the second nucleic acid molecule independently comprises an oligonucleotide between 6 and 14 nucleotides in length.

In embodiments, the first nucleic acid molecule and the second nucleic acid molecule of the circularizing domain are, independently, 6, 7, 8, 9, 10, 11, 12, 13, 14, 15, 16, 17, 18, 19, 20, 21, 22, 23, 24, 25, 26, 27, 28, 29, or 30 nucleotides in length.

In embodiments, the first nucleic acid molecule and the second nucleic acid molecule are the same length. In embodiments, the first nucleic acid molecule and the second nucleic acid molecule are different lengths.

As used herein, the term “polarity” refers to the concept of directionality in primary structure (e.g., 3′→5′ and 5′→3′ in the case of DNA and RNA, or N-terminal→C-terminal (or vice versa) in the case of PNAs). In the case where the CPNs of the invention comprise a first nucleic acid molecule and a second nucleic acid molecule, which, for example, hybridize by Watson-Crick base pairing in anti-parallel fashion, the first nucleic acid molecule can be in the 5′→3′ (or 2′) configuration and the sequence of nucleotides to which it is complementary in the second nucleic acid molecule can be in the 3′ (or 2′)→5′ configuration. Alternatively, the first nucleic acid molecule can be in the 3′ (or 2′)→5′ configuration and the sequence of nucleotides to which it is complementary in the second nucleic acid molecule will be in the 5′→3′ (or 2′) configuration.

The specific internal linkages of the first nucleic acid molecule and the second nucleic acid molecule is not critical so long as these two segments of the circularizing domain hybridize.

In an embodiment, the 5′ end of the oligonucleotide of the functional domain and the 5′ end of the first nucleic acid molecule are linked via a 5′-5′ linkage and the 3′ end of the oligonucleotide of the functional domain and the 5′ end of the second nucleic acid molecule are linked via a 3′-5′ linkage.

In an embodiment, the 5′ end of the oligonucleotide of the functional domain and the 3′ end of the first nucleic acid molecule are linked via a 5′-3′ linkage and the 3′ end of the oligonucleotide of the functional domain and the 3′ end of the second nucleic acid molecule are linked via a 3′-3′ linkage.

In embodiments, the first nucleic acid molecule is at least 95% complementary to the second nucleic acid molecule. In embodiments, the first nucleic acid molecule is at least 97% complementary to the second nucleic acid molecule. In embodiments, the first nucleic acid molecule is at least 98% complementary to the second nucleic acid molecule. In embodiments, the first nucleic acid molecule is at least 99% complementary to the strand of the second nucleic acid molecule. In embodiments, the first nucleic acid molecule is at least 100% complementary to the second nucleic acid molecule.

In embodiments, the oligonucleotide of the functional domain is linked to the first nucleic acid molecule and/or the second nucleic acid molecule through the linker segment. In embodiments, the linker segment is a direct bond, a nucleotide or oligonucleotide between 2 and 5 nucleotides in length, or other chemical moiety, or combinations thereof. In some embodiments, the linker segment can be cleavable.

In embodiments, the linker segment between the oligonucleotide and the first nucleic acid molecule is the same as the linker segment between the oligonucleotide and the second nucleic acid molecule. In embodiments, the linker segment is a direct bond.

In embodiments, the linker segment between the oligonucleotide and the first nucleic acid molecule is different than the linker segment between the oligonucleotide and the second nucleic acid molecule.

The only limitation on the linker segment is that it does not eliminate the essential functions of the CPN, namely (a) the ability of the CPN to form an intramolecular circular structure under the conditions of interest (e.g., physiological conditions) and (b) the ability of the functional domain to carry out its intended function.

Preferred “other chemical moiety” linkers include, but are not limited to, C₂-C₆ alkyl, ethylene glycol, tri (ethylene glycol), tetra (ethylene glycol), penta (ethylene glycol), hexa (ethylene glycol) and —NH(CH₂)ₙNH—, wherein n is 2, 3, 4, 5, or 6. Alternatively, the linker segment can be a combination of the foregoing.

In a preferred embodiment, the linker is a direct bond.

In an embodiment, the linker is ethylene glycol. In embodiments, the linker is a C₂-C₆ alkyl. In embodiments, the linker is a C₂ alkyl. In embodiments, the linker is a C₃ alkyl. In embodiments, the linker is a C₄ alkyl. In embodiments, the linker is a C₅ alkyl. In embodiments, the linker is a C₆ alkyl.

In an embodiment, the linker is selected from a lipid, fatty acid, antibody, galnac, peptide, or protein.

When every base in at least one strand of a pair of nucleic acids is found opposite its complementary base pair, such strand is considered fully complementary to its sequence in the other strand. When one, or more, bases of such a strand is found in a position where it is opposite any other base excepting its complementary base pair, that base is considered “mis-matched” and the strand is considered partially complementary. Accordingly, strands can be varying degrees of partially complementary (e.g., 0%<x<100% complementary), until no bases align, at which point they are non-complementary (e.g., 0% complementary). As is readily understood and recognized by one of skill in the art, full (i.e., complete, 100%) complementarity is not required for hybridization of strands of nucleic acids (e.g., oligonucleotides, antisense or otherwise).

Examples of first and second nucleic acid molecules of the circularizing domains useful in the CPN oligonucleotides described herein include, but are not limited to, the nucleic acids of Table 2.

TABLE 2

| SEQ ID NO: | Sequence |
|---|---|
| SEQ ID NO: 8 | 5′-ctagtaccgtaa-3′ |
| SEQ ID NO: 9 | 5′-CTAGTACCGTAA-3′ |
| SEQ ID NO: 10 | 5′-ttacggtactag-3′ |
| SEQ ID NO: 11 | 5′-TTACGGTACTAG-3′ |
| SEQ ID NO: 12 | 5′-cgtatctagtaccgtaa-3′ |
| SEQ ID NO: 13 | 5′-CGTATCTAGTACCGTAA-3′ |
| SEQ ID NO: 14 | 5′-ttacgtactagatacg-3′ |
| SEQ ID NO: 15 | 5′-TTACGTACTAGATACG-3′ |
| SEQ ID NO: 16 | 5′-T₁C₁C₁C₁T₁A₁G₁T₁G₁T₁C₁T₁A₁A₁-3′ |
| SEQ ID NO: 17 | 5′-T₁C₁C₁C₁T₁A₁G₁T₁G₁T₁-3′ |
| SEQ ID NO: 18 | 5′-T₁C₁C₁C₁T₁A₁G₁T₁-3′ |
| SEQ ID NO: 19 | 5′T₁T₁A₁G₁A₁C₁A₁C₁T₁A₁G₁G₁G₁A₁-3′ |
| SEQ ID NO: 20 | 5′-A₁C₁A₁C₁T₁A₁G₁G₁G₁A₁-3′ |
| SEQ ID NO: 21 | 5′-A₁C₁T₁A₁G₁G₁G₁A₁-3′ |
| SEQ ID NO: 22 | 5′-A₁G₁T₁C₁A₁T₁C₁T₁A₁G₁A₁C₁A₁T₁-3′ |
| SEQ ID NO: 23 | 5′-A₁G₁T₁C₁A₁T₁C₁T₁A₁G₁-3′ |
| SEQ ID NO: 24 | 5′-A₁G₁T₁C₁A₁T₁C₁-3′ |
| SEQ ID NO: 25 | 5′-A₁T₁G₁T₁C₁T₁A₁G₁A₁T₁G₁A₁C₁T₁-3′ |
| SEQ ID NO: 26 | 5′-A₁T₁G₁T₁C₁T₁A₁G₁A₁T₁-3′ |
| SEQ ID NO: 27 | 5′-A₁T₁G₁T₁C₁T₁-3′ |
| SEQ ID NO: 28 | 5′-T₁T₁A₁G₁a₁c₁a₁c₁t₁a₁g₁g₁g₁a₁-3′ |
| SEQ ID NO: 29 | 5′-a₁c₁a₁c₁t₁a₁g₁g₁g₁a₁-3′ |
| SEQ ID NO: 30 | 5′-a₁c₁t₁a₁g₁g₁g₁a₁-3′ |

Uppercase C/G/T/A is DNA with phosphorothioate linkage; lowercase c/g/t/a is RNA with phosphodiester linkage; C₁/G₁/T₁/A₁ is DNA with phosphodiester linkage; lowercase c₁/g₁/t₁/a₁ is RNA with phosphodiester linkage

In embodiments, circular prodrug nucleic acid according to the invention is part of a pharmaceutical composition comprising a pharmaceutically acceptable carrier.

The pharmaceutical composition comprising the circular prodrug nucleic acid of the invention may further comprise any other agent or therapy useful for treating or preventing a disease or condition and does not diminish the function of the circular prodrug nucleic acid according to the invention. Agent(s) useful for treating or preventing the disease or condition includes, but is not limited to, small molecules, peptides, vaccines, antigens, antibodies, preferably monoclonal antibodies, cytotoxic agents, kinase inhibitors, allergens, antibiotics, siRNA molecules, antisense oligonucleotides, TLR antagonist (e.g. antagonists of TLR3 and/or TLR7 and/or antagonists of TLR8 and/or antagonists of TLR9), chemotherapeutic agents (both traditional chemotherapy and modem targeted therapies), targeted therapeutic agents, activated cells, peptides, proteins, gene therapy vectors, peptide vaccines, protein vaccines, DNA vaccines, adjuvants, and co-stimulatory molecules (e.g. cytokines, chemokines, protein ligands, trans-activating factors, peptides or peptides comprising modified amino acids), or combinations thereof. Alternatively, the circular prodrug nucleic acid according to the invention can be administered in combination with other compounds (for example formulated with lipids or liposomes, and conjugated to peptides, antibodies, or small molecules) to enhance the specificity or magnitude of the gene expression modulation of the circular prodrug nucleic acid according to the invention.

### Functional Domains

In any of the embodiments described herein the functional domain of the CPN can be an oligonucleotide as further described below. The first nucleic acid molecule and the second nucleic acid molecule of the circularizing domain and the linker segment of the CPN is as described above unless otherwise noted.

As described above, the functional domain includes, but is not limited to, an oligonucleotide selected from an antisense oligonucleotide, a microRNA (miRNA), a siRNA, a piRNA, a hnRNA, a ncRNA, a snRNA, a miRNA mimic, a sgRNA, an esiRNA, a shRNA, a lncRNA, a mRNA, an aptamer, a CRISPR-based system, an adenosine deaminase acting on RNA (ADAR) system, or a splicing oligonucleotide. In embodiments, the functional domain includes, but is not limited to, an oligonucleotide selected from an immunostimulatory oligonucleotide or an immune-inhibitory oligonucleotide, also referred to as an immune antagonist oligonucleotide.

In embodiments, wherein the oligonucleotide of the functional domain is an oligonucleotide of a CRISPR-based system or an adenosine deaminase acting on RNA (ADAR) system, then the portion of the oligonucleotide that is complementary to the target RNA (complementary domain) is at least 90% complementary over its entire length to a portion of the target RNA, preferably at least 95% complementary, preferably at least 97% complementary, preferably at least 98% complementary, preferably at least 99% complementary, or preferably at least 100% complementary.

In embodiments, the oligonucleotide of the functional domain comprises at least one phosphorothioate internucleotide linkage. In embodiments, at least half of the internucleotide linkages are phosphorothioate. In embodiments, all of the internucleotide linkages are phosphorothioate.

In embodiments, the oligonucleotide of the functional domain is single-stranded.

In embodiments, the oligonucleotide of the functional domain is at least 90% complementary over its entire length to a portion of a target RNA. In embodiments, the oligonucleotide of the functional domain is at least 95% complementary over its entire length to a portion of the target RNA. In embodiments, the oligonucleotide of the functional domain is at least 97% complementary over its entire length to a portion of the target RNA. In embodiments, the oligonucleotide of the functional domain is at least 98% complementary over its entire length to a portion of the target RNA. In embodiments, the oligonucleotide of the functional domain is at least 99% complementary over its entire length to a portion of the target RNA. In embodiments, the oligonucleotide of the functional domain is at least 100% complementary over its entire length to a portion of the target RNA.

In embodiments, the target RNA may be an mRNA, pre-mRNA, ncRNA, lncRNA, or microRNA. In embodiments, the target RNA is mRNA.

Examples of gene modulating oligonucleotides suitable for use in the functional domain of a circular prodrug nucleic acids of the invention include, but are not limited to, the gene modulating oligonucleotides of Table 1. Functional domain oligonucleotides directed to any other sequence within the target RNA or to any other target RNA of interest are well within the skill of one in the art.

TABLE 1

| Target | Sequence (SEQ ID NOs) |
|---|---|
| PCSK9 | 5′-GGTCTCCTCCATCAGCACC-3′ (SEQ ID NO: 1) |
| DMD | 5′-G₁G₁C₁C₁A₁A₁A₁C₁C₁U₁C₁G₁G₁C₁U₁U₁A₂C₂C₂T₂-3′ (SEQ ID NO: 2) |
| TTR | 5′-TCTTGGTTACATGAAATCCC-3′ (SEQ ID NO: 3) |
| ANGPLT3 | 5′-GGACATTGCCAGTAATCGCA-3′ (SEQ ID NO: 4) |
| Spinraza | 5′-T₁C₁A₁C₁T₁T₁T₁C₁A₁T₁A₁A₁T₁G₁C₁T₁G₁G₁-3′ (SEQ ID NO: 5) |
| ApoL1 | 5′-TGCTCCGTTGGTGCTTGTTC-3′ (SEQ ID NO: 6) |
| SOD1 | 5′-CAGGATACATTTCTACAGCT-3′ (SEQ ID NO: 7) |
| SOD1 | 5′-C₃A₃G₃G₃A₃T₃A₃C₃ATTTCTACAGCT-3′ (SEQ ID NO: 31) |
| APOC3 | 5′-GCTTCTTGTCCAGCTTTATT-3 (SEQ ID NO: 32) |
| APOC3 | 5′-G₃C₃T₃T₃C₃TTGTCCAGCTT₃T₃A₃T₃T₃-3′ (SEQ ID NO: 33) |
| APOC3 | 5′-G₃C₃T₃T₃C₃T₃T₃G₃TCCAGCTTTATT-3′ (SEQ ID NO: 34) |
| MAPT | 5′-C₃C₃G₃T₃T₃T₃T₃CTTACCACCCT-3′ (SEQ ID NO: 35) |
| ApoB-100 | 5′-GCCTCAGTCTGCTTCGCACC-3′ (SEQ ID NO: 38) |
| APOC3 | 5′-AGCTTCTTGTCCAGCTTTAT-3′ (SED ID NO: 39) |
| PTP-1b | 5′-GCTCCTTCCACTGATCCTGC-3′ (SED ID NO: 40) |
| PCSK9 | 5′-CCTTGGCCACGCCGGCATCC-3′ (SED ID NO: 41) |
| STAT3 | 5′-CTATTTGGATGTCAGC-3′ (SED ID NO: 42) |
| Kallikrein B1 | 5′-TGCAAGTCTCTTGGCAAACA-3′ (SED ID NO: 43) |
| DGAT2 | 5′-TGCCATTTAATGAGCTTCAC-3′ (SED ID NO: 44) |
| SMN2 | 5′-TCACTTTCATAATGCTGG-3′ (SED ID NO: 45) |
| FUS | 3′-GCAAUGTCACCTTTCAUACC-5′ (SED ID NO: 46) |
| APO(a) | 5′-TGCTCCGTTGGTGCTTGTTC-3′ (SED ID NO: 47) |
| HTT | 5′-CUCAGTAACATTGACACCAC-3′ (SED ID NO: 48) |
| HBV | 5′-GCAGAGGTGAAGCGAAGTGC-3′ (SED ID NO: 49) |
| Angiotensinogen | 5′-GGGCCTGTTTAGTCGC-3′ (SED ID NO: 50) |
| Factor XI | 5′-ACGGCATTGGTGCACAGTTT-3′ (SED ID NO: 51) |
| TMPRSS6 | 5′-CTTTATTCCAAAGGGCAGCT-3′ (SED ID NO: 52) |
| MAPT | 5′-CCGTTTTCTTACCACCCT-3′ (SED ID NO: 53) |
| PTP1B (PTPN1) | 5′-ATGGTTTATTCCATGGCCA-3′ (SED ID NO: 54) |
| HTT | 5′-CUUUCAGACACGGGAACACGG-3′ (SED ID NO: 55) |
| ATXN2 | 5′-CTTCACATTTCGATCCAACA-3′ (SED ID NO: 56) |
| SCNIA | 5′-AGTTGGAGCAAGATTATC-3′ (SED ID NO: 57) |
| DMD | 5′-CAATGCCATCCTGGAGTTCCTG-3′ (SED ID NO: 58) |
| DMD | 5′-GTTGCCTCCGGTTCTGAAGGTGTTC-3′ (SED ID NO: 59) |
| DMD | 5′-CTCCAACATCAAGGAAGATGGCATTTCTAG-3′ (SED ID NO: 60) |
| ApoB-100 | 5′-G₁C₁C₁T₁C₁A₁G₁T₁CTGCTTCGCACC-3′ (SEQ ID NO: 61) |
| ApoB-100 | 5′-G₁C₁C₁T₁C₁A₁G₁T₁C₁TGCTTCGCACC-3′ (SEQ ID NO: 62) |
| ApoB-100 | 5′-G₃C₃C₃T₃C₃A₃G₃T₃CTGCTTCGCACC-3′ (SEQ ID NO: 63) |
| ApoB-100 | 5′-G₃C₃C₃T₃C₃A₃G₃T₃C₃TGCTTCGCACC-3′ (SEQ ID NO: 64) |
| TTR | 5′-T₁C₁T₁T₁G₁G₁T₁T₁ACATGAAATCCC-3′ (SEQ ID NO: 65) |
| TTR | 5′-T₁C₁T₁T₁G₁G₁T₁T₁A₁CATGAAATCCC-3′ (SEQ ID NO: 66) |
| TTR | 5′-T₃C₃T₃T₃G₃G₃T₃T₃ACATGAAATCCC-3′ (SEQ ID NO: 67) |
| TTR | 5′-T₃C₃T₃T₃G₃G₃T₃T₃A₃CATGAAATCCC-3′ (SEQ ID NO: 68) |
| APOC3 | 5′-A₁G₁C₁T₁T₁C₁T₁T₁GTCCAGCTTTAT-3′ (SEQ ID NO: 69) |
| APOC3 | 5′-A₁G₁C₁T₁T₁C₁T₁T₁G₁TCCAGCTTTAT-3′ (SEQ ID NO: 70) |
| APOC3 | 5′-A₃G₃C₃T₃T₃C₃T₃T₃GTCCAGCTTTAT-3′ (SEQ ID NO: 71) |
| APOC3 | 5′-A₃G₃C₃T₃T₃C₃T₃T₃G₃TCCAGCTTTAT-3′ (SEQ ID NO: 72) |
| PTP-1b | 5′-G₁C₁T₁C₁C₁T₁T₁C₁CACTGATCCTGC-3′ (SEQ ID NO: 73) |
| PTP-1b | 5′-G₁C₁T₁C₁C₁T₁T₁C₁C₁ACTGATCCTGC-3′ (SEQ ID NO: 74) |
| PTP-1b | 5′-G₃C₃T₃C₃C₃T₃T₃C₃CACTGATCCTGC-3′ (SEQ ID NO: 75) |
| PTP-1b | 5′-G₃C₃T₃C₃C₃T₃T₃C₃C₃ACTGATCCTGC-3′ (SEQ ID NO: 76) |
| PCSK9 | 5′-C₁C₁T₁T₁G₁G₁C₁C₁ACGCCGGCATCC-3′ (SEQ ID NO: 77) |
| PCSK9 | 5′-C₁C₁T₁T₁G₁G₁C₁C₁A₁CGCCGGCATCC-3′ (SEQ ID NO: 78) |
| PCSK9 | 5′-C₃C₃T₃T₃G₃G₃C₃C₃ACGCCGGCATCC-3′ (SEQ ID NO: 79) |
| PCSK9 | 5′-C₃C₃T₃T₃G₃G₃C₃C₃A₃CGCCGGCATCC-3′ (SEQ ID NO: 80) |
| STAT3 | 5′-C₁T₁A₁T₁TTGGATGTCAGC-3′ (SEQ ID NO: 81) |
| STAT3 | 5′-C₁T₁A₁T₁T₁TGGATGTCAGC-3′ (SEQ ID NO: 82) |
| STAT3 | 5′-C₃T₃A₃T₃TTGGATGTCAGC-3′ (SEQ ID NO: 83) |
| STAT3 | 5′-C₃T₃A₃T₃T₃TGGATGTCAGC-3′ (SEQ ID NO: 84) |
| ANGPTL3 | 5′-G₁G₁A₁C₁A₁T₁T₁G₁CCAGTAATCGCA-3′ (SEQ ID NO: 85) |
| ANGPTL3 | 5′-G₁G₁A₁C₁A₁T₁T₁G₁C₁CAGTAATCGCA-3′ (SEQ ID NO: 86) |
| ANGPTL3 | 5′-G₃G₃A₃C₃A₃T₃T₃G₃CCAGTAATCGCA-3′ (SEQ ID NO: 87) |
| ANGPTL3 | 5′-G₃G₃A₃C₃A₃T₃T₃G₃C₃CAGTAATCGCA-3′ (SEQ ID NO: 88) |
| Kallikrein B1 | 5′-T₁G₁C₁A₁A₁G₁T₁C₁TCTTGGCAAACA-3′ (SEQ ID NO: 89) |
| Kallikrein B1 | 5′-T₁G₁C₁A₁A₁G₁T₁C₁T₁CTTGGCAAACA-3′ (SEQ ID NO: 90) |
| Kallikrein B1 | 5′-T₃G₃C₃A₃A₃G₃T₃C₃TCTTGGCAAACA-3′ (SEQ ID NO: 91) |
| Kallikrein B1 | 5′-T₃G₃C₃A₃A₃G₃T₃C₃T₃CTTGGCAAACA-3′ (SEQ ID NO: 92) |
| DGAT2 | 5′-T₁G₁C₁C₁A₁T₁T₁T₁AATGAGCTTCAC-3′ (SEQ ID NO: 93) |
| DGAT2 | 5′-T₁G₁C₁C₁A₁T₁T₁T₁A₁ATGAGCTTCAC-3′ (SEQ ID NO: 94) |
| DGAT2 | 5′-T₃G₃C₃C₃A₃T₃T₃T₃AATGAGCTTCAC-3′ (SEQ ID NO: 95) |
| DGAT2 | 5′-T₃G₃C₃C₃A₃T₃T₃T₃A₃ATGAGCTTCAC-3′ (SEQ ID NO: 96) |
| SOD1 | 5′-C₁A₁G₁G₁A₁T₁A₁C₁ATTTCTACAGCT-3′ (SEQ ID NO: 97) |
| SOD1 | 5′-C₁A₁G₁G₁A₁T₁A₁CATTTCTACAGCT-3′ (SEQ ID NO: 98) |
| SOD1 | 5′-C₃A₃G₃G₃A₃T₃A₃C₃ATTTCTACAGCT-3′ (SEQ ID NO: 99) |
| SOD1 | 5′-C₃A₃G₃G₃A₃T₃A₃CATTTCTACAGCT-3′ (SEQ ID NO: 100) |
| SOD1 | 5′-C₃A₄G₃G₄A₃T₄A₃C₃ATTTCTACAGCT-3′ (SEQ ID NO: 101) |
| SOD1 | 5′-C₃A₄G₃G₄A₃T₄A₃CATTTCTACAGCT-3′ (SEQ ID NO: 102) |
| SOD1 | 5′-C₁A₁G₁G₁A₁T₁A₁C₁ATTTCTACAGCT-3′ (SEQ ID NO: 103) |
| SOD1 | 5′-C₁A₁G₁G₁A₁T₁A₁CATTTCTACAGCT-3′ (SEQ ID NO: 104) |
| SOD1 | 5′-C₃A₃G₃G₃A₃T₃A₃C₃ATTTCTACAGCT-3′ (SEQ ID NO: 105) |
| SOD1 | 5′-C₃A₃G₃G₃A₃T₃A₃CATTTCTACAGCT-3′ (SEQ ID NO: 106) |
| SOD1 | 5′-C₃A₄G₃G₄A₃T₄A₃C₃ATTTCTACAGCT-3′ (SEQ ID NO: 107) |
| SOD1 | 5′-C₃A₄G₃G₄A₃T₄A₃CATTTCTACAGCT-3′ (SEQ ID NO: 108) |
| FUS | 5′-C₁C₁A₁U₁A₁C₁T₁T₁TCCACTGUAACG-3′ (SEQ ID NO: 109) |
| FUS | 5′-C₃C₃A₃U₃A₃C₃T₃T₃TCCACTGUAACG-3′ (SEQ ID NO: 110) |
| FUS | 5′-C₃C₃A₃U₃A₃C₃T₃T₃T₃CCACTGUAACG-3′ (SEQ ID NO: 111) |
| APO(a) | 5′-T₁G₁C₁T₁C₁C₁G₁T₁T₁GGTGCTTGTTC-3′ (SEQ ID NO: 112) |
| APO(a) | 5′-T₁G₁C₁T₁C₁C₁G₁T₁TGGTGCTTGTTC-3′ (SEQ ID NO: 113) |
| APO(a) | 5′-T₃G₃C₃T₃C₃C₃G₃T₃T₃GGTGCTTGTTC-3′ (SEQ ID NO: 114) |
| APO(a) | 5′-T₃G₃C₃T₃C₃C₃G₃T₃TGGTGCTTGTTC-3′ (SEQ ID NO: 115) |
| HTT | 5′-C₁U₁C₁A₁G₁T₁A₁A₁C₁ATTGACACCAC-3′ (SEQ ID NO: 116) |
| HTT | 5′-C₁U₁C₁A₁G₁T₁A₁A₁C₁A₁TTGACACCAC-3′ (SEQ ID NO: 117) |
| HTT | 5′-C₃U₃C₃A₃G₃T₃A₃A₃C₃A₃TTGACACCAC-3′ (SEQ ID NO: 118) |
| HTT | 5′-C₃U₃C₃A₃G₃T₃A₃A₃C₃ATTGACACCAC-3′ (SEQ ID NO: 119) |
| HBV | 5′-G₁C₁A₁G₁A₁G₁G₁T₁G₁AAGCGAAGTGC-3′ (SEQ ID NO: 120) |
| HBV | 5′-G₁C₁A₁G₁A₁G₁G₁T₁GAAGCGAAGTGC-3′ (SEQ ID NO: 121) |
| HBV | 5′-G₃C₃A₃G₃A₃G₃G₃T₃G₃AAGCGAAGTGC-3′ (SEQ ID NO: 122) |
| HBV | 5′-G₃C₃A₃G₃A₃G₃G₃T₃GAAGCGAAGTGC-3′ (SEQ ID NO: 123) |
| Angiotensinogen | 5′-G₁G₁G₁C₁C₁TGTTTAGTCGC-3′ (SEQ ID NO: 124) |
| Angiotensinogen | 5′-G₁G₁G₁C₁CTGTTTAGTCGC-3′ (SEQ ID NO: 125) |
| Angiotensinogen | 5′-G₃G₃G₃C₃C₃TGTTTAGTCGC-3′ (SEQ ID NO: 126) |
| Angiotensinogen | 5′-G₃G₃G₃C₃CTGTTTAGTCGC-3′ (SEQ ID NO: 127) |
| Angiotensinogen | 5′-C₁A₁C₁A₁A₁A₁C₁A₁A₁GCTGGTCGGTT-3′ (SEQ ID NO: 128) |
| Angiotensinogen | 5′-C₁A₁C₁A₁A₁A₁C₁A₁AGCTGGTCGGTT-3′ (SEQ ID NO: 129) |
| Angiotensinogen | 5′-C₃A₃C₃A₃A₃A₃C₃A₃A₃GCTGGTCGGTT-3′ (SEQ ID NO: 130) |
| Angiotensinogen | 5′-C₃A₃C₃A₃A₃A₃C₃A₃AGCTGGTCGGTT-3′ (SEQ ID NO: 131) |
| Factor XI | 5′-A₁C₁G₁G₁C₁A₁T₁T₁GGTGCACAGTTT-3′ (SEQ ID NO: 132) |
| Factor XI | 5′-A₁C₁G₁G₁C₁A₁T₁T₁G₁GTGCACAGTTT-3′ (SEQ ID NO: 133) |
| Factor XI | 5′-A₃C₃G₃G₃C₃A₃T₃T₃GGTG₃CACAGTTT-3′ (SEQ ID NO: 134) |
| Factor XI | 5′-A₃C₃G₃G₃C₃A₃T₃T₃G₃GTGCACAGTTT-3′ (SEQ ID NO: 135) |
| SMN2 | 5′-T₁C₁A₁C₁T₁T₁T₁C₁A₁T₁A₁A₁T₁G₁CTGG-3′ (SEQ ID NO: 136) |
| SMN2 | 5′-T₃C₃A₃C₃T₃T₃T₃C₃A₃T₃A₃A₃T₃G₃CTGG-3′ (SEQ ID NO: 137) |
| TMPRSS6 | 5′-C₁T₁T₁T₁A₁T₁T₁C₁CAAAGGGCAGCT-3′ (SEQ ID NO: 138) |
| TMPRSS6 | 5′-C₁T₁T₁T₁A₁T₁T₁C₁C₁AAAGGGCAGCT-3′ (SEQ ID NO: 139) |
| TMPRSS6 | 5′-C₃T₃T₃T₃A₃T₃T₃C₃CAAAGGGCAGCT-3′ (SEQ ID NO: 140) |
| TMPRSS6 | 5′-C₃T₃T₃T₃A₃T₃T₃C₃C₃AAAGGGCAGCT-3′ (SEQ ID NO: 141) |
| MAPT | 5′-C₁C₁G₁T₁T₁T₁T₁CTTACCACCCT-3′ (SEQ ID NO: 142) |
| MAPT | 5′-C₁C₁G₁T₁T₁T₁TCTTACCACCCT-3′ (SEQ ID NO: 143) |
| MAPT | 5′-C₃C₃G₃T₃T₃T₃T₃CTTACCACCCT-3′ (SEQ ID NO: 144) |
| MAPT | 5′-C₃C₃G₃T₃T₃T₃TCTTACCACCCT-3′ (SEQ ID NO: 145) |
| PTP1B (PTPN1) | 5′-A₁T₁G₁G₁T₁T₁T₁A₁TTCCATGGCCA-3′ (SEQ ID NO: 146) |
| PTP1B (PTPN1) | 5′-A₁T₁G₁G₁T₁T₁T₁ATTCCATGGCCA-3′ (SEQ ID NO: 147) |
| PTP1B (PTPN1) | 5′-A₃T₃G₃G₃T₃T₃T₃A₃TTCCATGGCCA-3′ (SEQ ID NO: 148) |
| PTP1B (PTPN1) | 5′-A₃T₃G₃G₃T₃T₃T₃ATTCCATGGCCA-3′ (SEQ ID NO: 149) |
| HTT | 5′-C₁U₁U₁U₁C₁A₁G₁A₁C₁A₁CGGGAACACGG-3′ (SEQ ID NO: 150) |
| HTT | 5′-C₁U₁U₁U₁C₁A₁G₁A₁C₁ACGGGAACACGG-3′ (SEQ ID NO: 151) |
| HTT | 5′-C₃U₃U₃U₃C₃A₃G₃A₃C₃A₃CGGGAACACGG-3′ (SEQ ID NO: 152) |
| HTT | 5′-C₃U₃U₃U₃C₃A₃G₃A₃C₃ACGGGAACACGG-3′ (SEQ ID NO: 153) |
| ATXN2 | 5′-C₁T₁T₁C₁A₁C₁A₁T₁T₁TCGATCCAACA-3′ (SEQ ID NO: 154) |
| ATXN2 | 5′-C₁T₁T₁C₁A₁C₁A₁T₁TTCGATCCAACA-3′ (SEQ ID NO: 155) |
| ATXN2 | 5′-C₃T₃T₃C₃A₃C₃A₃T₃T₃TCGATCCAACA-3′ (SEQ ID NO: 156) |
| ATXN2 | 5′-C₃T₃T₃C₃A₃C₃A₃T₃TTCGATCCAACA-3′ (SEQ ID NO: 157) |
| SCNIA | 5′-A₁G₁T₁T₁G₁G₁A₁G₁C₁A₁A₁G₁A₁T₁TATC-3′ (SEQ ID NO: 158) |
| SCNIA | 5′-A₃G₃T₃T₃G₃G₃A₃G₃C₃A₃A₃G₃A₃T₃TATC-3′ (SEQ ID NO: 159) |
| DMD | 5′-C₁A₁A₁T₁G₁C₁C₁A₁T₁C₁C₁T₁G₁G₁A₁G₁T₁T₁CCTG-3′ (SEQ ID NO: 160) |
| DMD | 5′-C₃A₃A₃T₃G₃C₃C₃A₃T₃C₃C₃T₃G₃G₃A₃G₃T₃T₃CCTG-3′ (SEQ ID NO: 161) |
| DMD | 5′-G₁T₁T₁G₁C₁C₁T₁C₁C₁G₁G₁T₁T₁C₁T₁G₁A₁A₁G₁G₁T₁GTTC-3′ |
| | (SEQ ID NO: 162) |
| DMD | 5′-G₃T₃T₃G₃C₃C₃T₃C₃C₃G₃G₃T₃T₃C₃T₃G₃A₃A₃G₃G₃T₃GTTC-3′ |
| | (SEQ ID NO: 163) |
| DMD | 5′-C₁T₁C₁C₁A₁A₁C₁A₁T₁C₁A₁A₁G₁G₁A₁A₁G₁A₁T₁G₁G₁C₁A₁T₁T₁T₁CTAG-3′ |
| | (SEQ ID NO: 164) |
| DMD | 5′-C₃T₃C₃C₃A₃A₃C₃A₃T₃C₃A₃A₃G₃G₃A₃A₃G₃A₃T₃G₃G₃C₃A₃T₃T₃T₃CTAG-3′ |
| | (SEQ ID NO: 165) |
| FUS | 5′-C₁C₁A₁U₁A₁C₁T₁T₁T₁CCACTGUAACG-3′ (SEQ ID NO: 164) |
| HTT | 5′-C₃T₃C₃A₃G₃T₃A₃A₃C₃ATTGACACCAC-3′ (SEQ ID NO: 167) |
| HTT | 5′-C₃T₃C₃A₃G₃T₃A₃A₃C_{A}TTGACACCAC-3′ (SEQ ID NO: 168) |
| HTT | 5′-C₄T₃C₄A₃G₄T₃A₄A₃C₃ATTGACACCAC-3′ (SEQ ID NO: 169) |
| HTT | 5′-C₄T₃C₄A₃G₄T₃A₄A₃C₄atTGACACCAC-3′ (SEQ ID NO: 170) |
| HTT | 5′-C₄T₃C₄A₃G₄T₃A₄A₃C₃attGACACCAC-3′ (SEQ ID NO: 171) |
| HTT | 5′-C₄T₃C₄A₃G₄T₃A₄A₃C₃AttgACACCAC-3′ (SEQ ID NO: 172) |
| HTT | 5′-C₄T₃C₄A₃G₄T₃A₄A₃C₃atTGACACCAC-3′ (SEQ ID NO: 173) |
| HTT | 5′-C₄T₃C₄A₃G₄T₃A₄A₃C₃AttGACACCAC-3′ (SEQ ID NO: 174) |
| MAPT | 5′-C₃C₃G₃T₃T₃T₃T₃CTTACCACCCT-3 (SEQ ID NO: 175) |
| MAPT | 5′-C₃C₃G₄T₄T₄T₄T₃CTTACCACCCT-3′ (SEQ ID NO: 176) |
| SOD1 | 5′-C₄A₄G₃G₃A₃T₃A₃C₃ATTTCTACAGCT-3′ (SEQ ID NO: 177) |
| SOD1 | 5′-C₄A₄G₄G₃A₃T₃A₃C₃ATTTCTACAGCT-3′ (SEQ ID NO: 178) |
| SOD1 | 5′-C₄A₄G₄G₄A₃T₃A₃C₃ATTTCTACAGCT-3′ (SEQ ID NO: 179) |
| SOD1 | 5′-C₃A₄G₃G₄A₃TACATTTCTAC₄A₃G₄C₃T₃A₂C₂A₂G₂C₂T₂-3′ (SEQ ID NO: |
| | 180) |
| SOD1 | 5′-C₄A₄G₃G₃A₃T₃A₃C₃ATTTCTACAGCTA₂C₂A₂G₂C₂T₂-3′ (SEQ ID NO: |
| | 181) |
| SOD1 | 5′-C₄A₄G₄G₃A₃T₃A₃C₃ATTTCTACAGCTA₂C₂A₂G₂C₂T₂-3′ (SEQ ID NO: |
| | 182) |
| SOD1 | 5′-C₃A₃G₃G₃A₃T₃A₃C3A₃TTTCTACAGCTA₂G₂C₂A₂G₂G₂-3′ (SEQ ID NO: |
| | 183) |
| SOD1 | 5′-C₄A₄G₄G₃A₃T₃A₃C₃A₃TTTCTACAGCTA₂G₂C₂A₂G₂G₂-3′ (SEQ ID NO: |
| | 184) |
| SOD1 | 5′-C₃A₃G₃G₃A₃TACATTTCTAC₃A₃G₃C₃T₃-3′ (SEQ ID NO: 185) |
| SOD1 | 5′-C₃A₃G₃G₃A₃TACATTTCTAC₄A₃G₄C₃T₃T₂A₂G₂C₂A₂G₂G₂-3′ (SEQ ID |
| | NO: 186) |
| SOD1 | 5′-C₃A₄G₃G₄A₃TACATTTCTAC₄A₃G₄C₃T₃T₂A₂G₂C₂A₂G₂G₂-3′ (SEQ ID |
| | NO: 187) |
| APOC3 | 5′-G₃C₃T₃T₃C₃T₃T₃G₃T₃CCAGCTTTATT-3′ (SEQ ID NO: 188) |
| APOC3 | 5′-G₄C₄T₄T₃C₃T₃T₃G₃T₃CCAGCTTTATT-3′ (SEQ ID NO: 189) |
| APOC3 | 5′-G₃C₃T₃T₃CT₃T₃G₃T₃CCAGCTTTATT-3′ (SEQ ID NO: 190) |
| APOC3 | 5′-G₃C₃T₃T₃C₃T₃T₃G₃T₃CCAGCTTTATTG₂G₂G₂A₂G₂-3′ (SEQ ID NO: |
| | 191) |
| APOC3 | 5′-G₄C₄T₄T₃C₃T₃T₃G₃T₃CCAGCTTTATTG₂G₂G₂A₂G₂-3′ (SEQ ID NO: |
| | 192) |

Uppercase G/C/A/T- DNA with phosphorothioate linkage; lowercase a/t/c/g-RNA; G₁/C₁/A₁/U₁-2′OME or 2′ MOE ribonucleotide; A₂/T₂/C₂/G₂-DNA with phosphodiester linkage; lowercase a₁/t₁/c₁/g₁-RNA with phosphodiester linkage; G3/C3/A3/U3-2′ MOE ribonucleotide with phosphorothioate linkage; G₄/C₄/A₄/T₄/U₄-2′MOE phosphodiester linkage

### Inhibition of Gene Expression

In embodiments, the invention provides a circular prodrug nucleic acid (CPN) comprising a functional domain comprising a gene modulating oligonucleotide (i.e., an oligonucleotide that can modulate the expression of a target gene). Such oligonucleotides can include, but are not limited to, an antisense oligonucleotide, a microRNA (miRNA), a miRNA mimic, a piRNA, a hnRNA, a ncRNA, a siRNA, a snRNA, a sgRNA, an esiRNA, a shRNA, or a lncRNA.

In embodiments, the invention provides a circular prodrug nucleic acid (CPN) comprising a functional domain comprising an oligonucleotide between 15 and 45 nucleotides in length and complementary to target RNA; wherein the 5′-end of the oligonucleotide is linked (directly or through a linker segment) to a first nucleic acid molecule and the 3′-end of the oligonucleotide is linked to a second nucleic acid molecule; wherein the first nucleic acid molecule and the second nucleic acid molecule form a circularizing domain as described herein; wherein the oligonucleotide of the functional domain comprises a gene modulating oligonucleotide. In embodiments, the oligonucleotide of the functional domain is modified. In embodiments, the first nucleic acid molecule and the second nucleic acid molecule are independently 6 to 30 nucleotides in length, wherein the nucleotides of the first nucleic acid molecule and the second nucleic acid molecule are independently selected from RNA or DNA, and wherein the first nucleic acid molecule and the second nucleic acid molecule are complementary to and of opposite polarity to each other and hybridize to form a double-stranded section. In embodiments, the first nucleic acid molecule and the second nucleic acid molecule are not both DNA.

In embodiments, the modification of the oligonucleotide comprises at least one modified nucleobase, sugar and/or internucleotide linkage.

As shown herein, the CPNs of the invention comprising a gene modulating oligonucleotide as the functional domain surprisingly demonstrated increased potency. Furthermore, gene modulating oligonucleotide of the CPN lacks a free 5′ end, the CPN may be less inflammatory. This design permits antisense oligonucleotides to unfold to linear structure and to be active upon in situ activation in cells where the target RNA is expressed.

In embodiments, where the functional domain of the CPN is an antisense oligonucleotide, it is part of the circular prodrug until it is in the cytoplasm or the nucleus where the circularizing domain (made up of the first nucleic acid molecule and the second nucleic acid molecule) is cleaved by RNase H or Dicer, thereby linearizing the CPN and allowing for the antisense oligonucleotide of the functional domain to bind to the target RNA. The changes from circular form to linear form could be confirmed by RNase H and/or Dicer cleavage studies. In the linear form, the functional domain hybridizes (under physiological conditions, at a minimum) with the complementary target RNA to form a duplex. Depending on the intended mechanism of action of the functional domain, the duplex formed with the target RNA would lead to modulation of translation. For example, this duplex is a substrate for RNase H, and, in the presence of RNase H and under the proper conditions (e.g., physiological), the target RNA strand of the duplex will be cleaved by the RNase H, thereby preventing expression.

CPNs comprising an antisense oligonucleotide as the functional domain maintain activity in cell cultures. The advantage foreseen with these CPNs is that their formation of intramolecular circular structures allows for less interaction with non-targeted macromolecules (including nucleic acids and proteins), have reduced polyanionic-related side effect. Additionally, due to circular structure, these CPNs can escape from endosomes due to a lack of a free 5′ end of the oligonucleotide to interaction with pattern recognition receptors.

The oligonucleotides of the invention are isolated oligonucleotides. The term “isolated” means altered or removed from the natural state through human intervention. For example, an oligonucleotide naturally present in a living animal is not “isolated,” but a synthetic oligonucleotide, or an oligonucleotide partially or completely separated from the coexisting materials of its natural state is “isolated.” An isolated oligonucleotide can exist in substantially purified form, or can exist in a non-native environment such as, for example, a cell into which the oligonucleotide has been delivered. The oligonucleotides of the invention can comprise partially purified DNA and/or RNA, substantially pure DNA and/or RNA, synthetic DNA and/or RNA, or recombinantly produced DNA and/or RNA, as well as altered DNA and/or RNA that differs from naturally occurring DNA and/or RNA by the addition, deletion, substitution and/or alteration of one or more nucleotides. Such alterations can include addition of non-nucleotide material, such as to the end(s) of the oligonucleotide or to one or more internal nucleotides of the oligonucleotide, including modifications that make the oligonucleotide resistant to nuclease digestion.

The terms “microRNA,” “miRNA,” and “MiR” are interchangeable and refer to endogenous or artificial non-coding RNAs that can regulate gene expression. It is believed that miRNAs function via RNA interference. The design of such microRNAs is within the skill of ordinary artisans.

The terms “piRNA” and “Piwi-interacting RNA” are interchangeable and refer to a class of small RNAs involved in gene silencing. PiRNA molecules typically are between 26 and 31 nucleotides in length. The design of such piRNAs is within the skill of ordinary artisans.

### Splitmer

In embodiments, the antisense oligonucleotide of the functional domain is as described in WO2020/191177, which is incorporated herein by reference in its entirety. In embodiments, the antisense oligonucleotide of the functional domain is a modified oligonucleotide comprising or consisting of an antisense oligonucleotide compound 17 to 25 nucleotides in length, wherein the antisense oligonucleotide compound comprises a 3′ domain and a 5′ domain, which is contiguous with the 3′ domain, wherein the 3′ domain begins at the terminal nucleotide at the 3′ end and is 10 to 12 nucleotides in length and each nucleotide comprises a deoxyribonucleotide and a phosphodiester or phosphothioate internucleotide linkage or combinations thereof; and wherein the 5′ domain begins at the first nucleotide following the 3′ domain and continues to the terminal nucleotide at the 5′ end, wherein the 5′ domain comprises unmodified deoxyribonucleotides, unmodified ribonucleotides, modified deoxyribonucleotides, modified ribonucleotides, or combinations thereof, provided that the 5′ domain comprises at least 3 modified deoxyribonucleotide or modified ribonucleotide. The modified deoxyribonucleotides and/or modified ribonucleotides of the 5′ domain need not be consecutive. The modified deoxyribonucleotides and/or modified ribonucleotides of the 5′ domain prevent RNase H cleavage in the 5′ domain. In embodiments, the modified deoxyribonucleotide or modified ribonucleotide comprise a modified base, a modified sugar and/or modified backbone. In embodiments, the modified deoxyribonucleotide or modified ribonucleotide comprise a modified sugar and/or modified backbone.

In embodiments, at least half of the nucleotides of the 5′ domain comprise a modified deoxyribonucleotide or modified ribonucleotide comprising a modified sugar and/or backbone. In embodiments, at least half of the nucleotides of the 5′ domain comprise a modified ribonucleotide comprising a modified sugar and/or backbone. Such antisense oligonucleotides are referred to as “splitmer”.

In embodiments, all of the nucleotides of the 5′ domain comprise a modified deoxyribonucleotide or modified ribonucleotide comprising a modified sugar and/or backbone.

When less than all of the nucleotides of the 5′ domain are modified, the modified and unmodified nucleotides are arranged so that no more than 2 unmodified nucleotides in the 5′ domain are next to each other.

In embodiments, the splitmer of the functional domain comprises 17 to 25 linked nucleotides having at least 12 contiguous nucleobases complementary to an equal length portion of a target RNA.

In embodiments, the modified ribonucleotides of the splitmer comprise 2′-substituted nucleotides as described herein. In embodiments, the 2′-substituted nucleotides are selected from 2′ O-methyl ribonucleosides (2′-OME) or 2′-methoxyethyl ribonucleosides (2′-MOE).

In some embodiments, the 3′ domain comprises nucleotides at positions 1, 2, 3, 4, 5, 6, 7, 8, 9, 10, 11, and 12 from the 3′ end. In some embodiments, the 3′ domain comprises nucleotides at positions 1, 2, 3, 4, 5, 6, 7, 8, 9, 10, and 11 from the 3′ end. In some embodiments, the 3′ domain comprises nucleotides at positions 1, 2, 3, 4, 5, 6, 7, 8, 9, and 10 from the 3′ end.

In some embodiments, when the 3′ domain of the antisense oligonucleotide is 12 nucleotides in length, the antisense oligonucleotides of the invention are represented by Formula (I):

5′-NₘN₁₄N₁₃N₁₂N₁₁N₁₀N₉N₈N₇N₆N₅N₄N₃N₂N₁-3′

wherein
N is any nucleotide;
N₁₃ through Nₘ comprises the 5′ domain;
N₁ through N₁₂ comprises the 3′ domain; and
m is selected from 0, 1, 2, 3, 4, 5, 6, 7, 8, 9, 10, or 11.

In some embodiments, when the 3′ domain of the antisense oligonucleotide is 11 nucleotides in length, the antisense oligonucleotides of the invention are represented by Formula (Ia):

5′-NₘN₁₄N₁₃N₁₂N₁₁N₁₀N₉N₈N₇N₆N₅N₄N₃N₂N₁-3′

wherein
N is any nucleotide;
N₁₂ through Nₘ comprises the 5′ domain;
N₁ through N₁₁ comprises the 3′ domain; and
m is selected from 0, 1, 2, 3, 4, 5, 6, 7, 8, 9, 10, or 11.

In some embodiments, the antisense oligonucleotides of the invention are represented by Formula (Ib):

5′-NₘN₁₄N₁₃N₁₂N₁₁N₁₀N₉N₈N₇N₆N₅N₄N₃N₂N₁-3′

wherein
N is any nucleotide;
N₁₁ through Nₘ comprises the 5′ domain;
N₁ through N₁₀ comprises the 3′ domain; and
m is selected from 0, 1, 2, 3, 4, 5, 6, 7, 8, 9, 10, or 11.

In some embodiments, m is 0. In some embodiments, m is selected from 1, 2, 3, 4, 5, 6, or 7. In some embodiments, m is selected from 1, 2, 3, 4, 5, or 6. In some embodiments, m is selected from 1, 2, 3, 4, or 5. In some embodiments, m is selected from 1, 2, 3, or 4. In some embodiments, m is selected from 1, 2, or 3. In some embodiments, m is 1. In some embodiments, m is 2. In some embodiments, m is 3. In some embodiments, m is 4. In some embodiments, m is 5. In some embodiments, m is 6. In some embodiments, m is 7. In some embodiments, m is 8. In some embodiments, m is 9. In some embodiments, m is 10. In some embodiments, m is 11.

In embodiments, the splitmer antisense oligonucleotide compound of the functional domain is 17 to 25 nucleotides in length comprising at least 12 contiguous nucleobases complementary to an equal length portion of a target RNA sequence, wherein the antisense oligonucleotide compound comprises a 3′ domain and a 5′ domain, which is contiguous with the 3′ domain, wherein the 3′ domain begins at the terminal nucleotide at the 3′ end and is 10 to 12 nucleotides in length and each nucleotide comprises a deoxyribonucleotide and a phosphodiester or phosphothioate internucleotide linkage or combinations thereof; and wherein the 5′ domain begins at the first nucleotide following the 3′ domain and continues to the terminal nucleotide at the 5′ end, wherein the 5′ domain comprises unmodified deoxyribonucleotides, unmodified ribonucleotides, modified deoxyribonucleotides, modified ribonucleotides, or combinations thereof, provided that the 5′ domain comprises at least 3 modified deoxyribonucleotide or modified ribonucleotide, wherein the modified deoxyribonucleotides and/or modified ribonucleotides of the 5′ domain need not be consecutive; and wherein the modified deoxyribonucleotides and/or modified ribonucleotides of the 5′ domain prevent RNase H cleavage in the 5′ domain. In embodiments, the modified deoxyribonucleotide or modified ribonucleotide comprise a modified base, a modified sugar and/or modified backbone. In embodiments, the modified deoxyribonucleotide or modified ribonucleotide comprise a modified sugar and/or modified backbone.

In embodiments, at least half of the nucleotides of the 5′ domain comprise a modified deoxyribonucleotide or modified ribonucleotide comprising a modified sugar and/or backbone. In embodiments, at least half of the nucleotides of the 5′ domain comprise a modified ribonucleotide comprising a modified sugar and/or backbone.

In embodiments, all of the nucleotides of the 5′ domain comprise a modified deoxyribonucleotide or modified ribonucleotide comprising a modified sugar and/or backbone.

When less than all of the nucleotides of the 5′ domain are modified, the modified and unmodified nucleotides are arranged so that no more than 2 unmodified nucleotides in the 5′ domain are next to each other.

In embodiments, the 3′ domain is 12 nucleotides in length and comprises nucleotides at positions 1, 2, 3, 4, 5, 6, 7, 8, 9, 10, 11, and 12 from the 3′ end (position 1 is the 3′ end). In embodiments, the 3′ domain is 11 nucleotides in length and comprises nucleotides at positions 1, 2, 3, 4, 5, 6, 7, 8, 9, 10, and 11 from the 3′ end. In embodiments, the 3′ domain is 12 nucleotides in length and comprises nucleotides at positions 1, 2, 3, 4, 5, 6, 7, 8, 9, and 10 from the 3′ end.

In embodiments, the nucleotides of the 3′ domain comprise a natural nucleobase. In some embodiments, the nucleobases and sugars of the nucleotides of the 3′ domain of the antisense oligonucleotide according to the invention are unmodified. In this respect, the nucleobases and sugars of the nucleotides of the 3′ domain of the antisense oligonucleotide according to the invention are naturally occurring. Each of the nucleotides of the 3′ domain comprise a deoxyribonucleotide and a phosphodiester or phosphorothioate internucleotide linkage or combinations thereof. The nucleotides of the 3′-domain comprise natural deoxyribose sugar and phosphorothioate, phosphodiester or other phosphorus-based linkages or combinations thereof, which are known to activate RNase H.

In embodiments, at least one of the nucleotides of the 3′ domain comprises a modified nucleobase.

In embodiments, the nucleotides at the 9^{th} or 10^{th} positions from the 3′ end are not modified. In embodiments, the nucleotides at the 9^{th} and 10^{th} positions from the 3′ end are not modified. In embodiments, the nucleotide at the 11^{th} position from the 3′ end is not modified. In embodiments, the nucleotides at the 9^{th}, 10^{th}, and 11^{th} positions from the 3′ end are not modified. In embodiments, the nucleotide at the 12^{th} position from the 3′ end is not modified. In embodiments, the nucleotides at the 9^{th}, 10^{th}, 11^{th}, and 12^{th} positions from the 3′ end are not modified.

In embodiments, the oligonucleotide comprises at least one phosphorothioate internucleotide linkage. In embodiments, at least half of the internucleotide linkages are phosphorothioate. In embodiments, all of the internucleotide linkages are phosphorothioate. In embodiments, at least half of the internucleotide linkages are phosphodiester. In embodiments, all of the internucleotide linkages are phosphodiester.

In embodiments, the antisense oligonucleotide is single stranded.

As used here, the term “5′ domain” refers to the nucleotides beginning at the first nucleotide following the 3′ domain and goes to the 5′ end. The 5′ domain hybridizes to the target RNA but does not allow RNase H to excise the target RNA in this domain. The term “5′ domain” is generally 2 to 15 nucleotides in length and refers to the 11^{th} through the 25^{th} nucleotides (the 1^{st} nucleotide is the 3′ end), 12^{th} through the 25^{th} nucleotides, or 13^{th} through the 25^{th} nucleotides of the antisense oligonucleotide as measured from the 3′ end depending on the length of the 3′ domain. In embodiments, the 5′ domain refers to the 11^{th} nucleotide through the 5′ terminal nucleotide (the 1^{st} nucleotide is the terminal nucleotide at the 3′ end) of the splitmer oligonucleotide of the functional domain. In embodiments, the 5′ domain refers to the 12^{th} nucleotide through the 5′ terminal nucleotide of the splitmer oligonucleotide of the functional domain. In embodiments, the 5′ domain refers to the 13^{th} nucleotide through the 5′ terminal nucleotide of the splitmer oligonucleotide of the functional domain.

For example, an antisense oligonucleotide compound that is 20 nucleotides in length may comprise a 3′ domain from position 1 to position 12 and a 5′ domain from position 13 to position 20. The designation of the modified nucleotide is position-specific, as opposed to nucleotide-specific.

The 5′ domain comprises nucleotides having non-RNase H activating modifications such as modified sugars and/or modified backbones, which do not activate RNase H. In some embodiments, the 5′ domain comprises nucleotides comprising a modified sugar. In some embodiments, the 5′ domain comprises nucleotides comprising a modified backbone. In some embodiments, the 5′ domain comprises nucleotides comprising both a modified sugar and modified backbone. In embodiments, the modified backbone is a non-phosphorus-based backbone.

This design of antisense allows for targeted RNA cleavage at the specific sites towards the 5′ end of 3′ domain.

In any of these embodiments it is contemplated that at least half of the nucleotides of the 5′ domain comprise a backbone modification or substitution and/or a sugar modification or substitution. In some embodiments, the nucleotides at all positions within the 5′ domain comprises a backbone modification or substitution and/or a sugar modification or substitution. In one embodiment, the 5′ domain comprises at least four nucleotides comprising a modified backbone and/or sugar. In one embodiment, the 5′ domain comprises at least five nucleotides comprising a modified backbone and/or sugar. In one embodiment, the 5′ domain comprises at least six nucleotides comprising a modified backbone and/or sugar. In one embodiment, the 5′ domain comprises at least seven nucleotides comprising a modified backbone and/or sugar. In one embodiment, the 5′ domain comprises at least eight nucleotides comprising a modified backbone and/or sugar. In one embodiment, all of the nucleotides of the 5′ domain are nucleotides comprising a modified backbone and/or sugar.

It is specifically contemplated that embodiments discussed herein, in the context of a specific nucleotide and position, may be implemented with respect to a position relative to the 3′ end. For example, an antisense oligonucleotide with a modified nucleotide at position 13 refers to an antisense oligonucleotide having a modified nucleotide at position 13 from the 3′ end of the antisense oligonucleotide.

In embodiments, the antisense oligonucleotide of the functional domain is at least 90% complementary over its entire length to a portion of the target RNA.

### Gapmer

In embodiments, the oligonucleotide of the functional domain is a “gapmer”. As used herein, a gapmers is a chimeric antisense oligonucleotide that contains a central block of deoxynucleotide monomers sufficiently long to induce RNase H cleavage. Usually, the gapmers of the invention are directed against one or more mRNA encoding a target mRNA. The design of such gapmers is within the skill of ordinary artisans.

In embodiments, the antisense oligonucleotide of the functional domain is a modified oligonucleotide comprising or consisting of a region having a gapmer motif, which is defined by two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleosides having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer independently comprise 1-6 nucleosides. In certain embodiments, the wings of a gapmer independently comprise 1-5 nucleosides. In certain embodiments, the wings of a gapmer comprise the same number of nucleosides. In certain embodiments, the wings of a gapmer comprise 4 nucleosides. In certain embodiments, each nucleoside of each wing of a gapmer is a modified nucleoside.

In certain embodiments, the gap of a gapmer comprises 7-24 nucleosides. In certain embodiments, the gap of a gapmer comprises 7-18 nucleosides. In certain embodiments, the gap of a gapmer comprises 9-14 nucleosides. In certain embodiments, the gap of a gapmer comprises 7-23 nucleosides. In certain embodiments, the gap of a gapmer comprises 9 nucleosides. In certain embodiments, the gap of a gapmer comprises 10 nucleosides. In certain embodiments, the gap of a gapmer comprises 11 nucleosides. In certain embodiments, the gap of a gapmer comprises 13 nucleosides. In certain embodiments, the gap of a gapmer comprises 14 nucleosides. In certain embodiments, the gap of a gapmer comprises 17 nucleosides. In certain embodiments, the gap of a gapmer comprises 18 nucleosides. In certain embodiments, each nucleoside of the gap of a gapmer is an unmodified 2′-deoxy nucleoside.

In certain embodiments, the gapmer is a deoxy gapmer. In embodiments, the nucleosides on the gap side of each wing/gap junction are unmodified 2′-deoxy nucleosides and the nucleosides on the wing sides of each wing/gap junction are modified nucleosides. In certain embodiments, each nucleoside of the gap is an unmodified 2′-deoxy nucleoside. In certain embodiments, each nucleoside of each wing of a gapmer is a modified nucleoside.

Herein, the lengths (number of nucleosides) of the three regions of a gapmer may be provided using the notation [#of nucleosides in the 5′-wing]-[#of nucleosides in the gap]-[#of nucleosides in the 3′-wing]. Thus, a 5-10-5 gapmer consists of 5 linked nucleosides in each wing and 10 linked nucleosides in the gap. Where such nomenclature is followed by a specific modification, that modification is the modification in the wings and the gap nucleosides comprise unmodified deoxynucleosides sugars. Thus, a 5-11-5 MOE or OMe gapmer consists of 5 linked MOE or OMe modified nucleosides in the 5′-wing, 11 linked deoxynucleosides in the gap, and 5 linked MOE or OMe nucleosides in the 3′-wing.

In certain embodiments, modified oligonucleotides are 4-13-4 MOE or OMe gapmers. In certain embodiments, modified oligonucleotides are 5-11-5 MOE or OME gapmers. In certain embodiments, modified oligonucleotides are 3-15-3 BNA gapmers. In certain embodiments, modified oligonucleotides are 3-15-3 LNA gapmers.

In any of the embodiments described herein, modified oligonucleotides comprise or consist of a region having a fully modified sugar motif. In such embodiments, each nucleoside of the fully modified region of the modified oligonucleotide comprises a modified sugar moiety. In certain embodiments, each nucleoside of the entire modified oligonucleotide comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified comprises the same 2′-modification. In certain embodiments, the uniformly modified sugar motif is 12 to 30 nucleosides in length. In certain embodiments, each nucleoside of the uniformly modified sugar motif is a 2′-substituted nucleoside, a sugar surrogate, or a bicyclic nucleoside. In certain embodiments, each nucleoside of the uniformly modified sugar motif comprises either a 2′-OCH₂CH₂OCH₃ group or a 2′-OCH₃ group. In certain embodiments, modified oligonucleotides having at least one fully modified sugar motif may also have at least 1, at least 2, at least 3, or at least 4 2′-deoxynucleosides.

Examples of circular prodrug nucleic acids useful for gene silencing include, but are not limited to, the circular prodrug nucleic acids of Table 3. Circular prodrug nucleic acids with functional domains directed to any other target of interest are well within the skill of one in the art. All internucleotide linkages are phosphorothioate linkages unless otherwise noted. The SEQ ID NOs for the oligonucleotides of the circularizing domain and functional domain of the CPN compounds shown below are found in Tables 1 and 2 above.

TABLE 3

| Cmpd # | Target | Sequence |
|---|---|---|
| 1 | PCSK9 | 3′-AATGCCATGATC-5′-5′-GGTCTCCTCCATCAGCACC-3′-5′- |
| | | TTACGGTACTAG-3 |
| 2 | PCSK9 | 3′-AATGCCATGATC-5′-5′-GGTCTCCTCCATCAGCACC-3′-5′- |
| | | ttacggtactag-3 |
| 3 | PCSK9 | 3′-aatgccatgatc-5′-5′-GGTCTCCTCCATCAGCACC-3′-5′- |
| | | TTACGGTACTAG-3 |
| 4 | PCSK9 | 3′-aatgccatgatc-5′-5′-GGTCTCCTCCATCAGCACC-3′-5′-ttacggtactag-3 |
| 5 | DMD | 3′-AATGCCATGATC-5′-5′- |
| | | G₁G₁C₁C₁A₁A₁A₁C₁C₁U₁C₁G₁G₁C₁U₁U₁A₂C₂C₂T₂-3′-5′- |
| | | TTACGGTACTAG-3′ |
| 6 | DMD | 3′-AATGCCATGATC-5′-5′- |
| | | G₁G₁C₁C₁A₁A₁A₁C₁C₁U₁C₁G₁G₁C₁U₁U₁A₂C₂C₂T₂-3′-5′-ttacggtactag-3′ |
| 7 | DMD | 3′-aatgccatgatc-5′-5′-G₁G₁C₁C₁A₁A₁A₁C₁C₁U₁C₁G₁G₁C₁U₁U₁A₂C₂C₂T₂-3′- |
| | | 5′-TTACGGTACTAG-3′ |
| 8 | DMD | 3′-aatgccatgatc-5′-5′-G₁G₁C₁C₁A₁A₁A₁C₁C₁U₁C₁G₁G₁C₁U₁U₁A₂C₂C₂T₂-3′- |
| | | 5′-ttacggtactag-3′ |
| 9 | TTR | 3′-AATGCCATGATCTATGC-5′-5′-TCTTGGTTACATGAAATCCC-3′- |
| | | 5′-TTACGTACTAGATACG-3′ |
| 10 | TTR | 3′-AATGCCATGATCTATGC-5′-5′-TCTTGGTTACATGAAATCCC-3′- |
| | | 5′-ttacgtactagatacg-3′ |
| 11 | TTR | 3′-aatgccatgatctatgc-5′-5′-TCTTGGTTACATGAAATCCC-3′-5′- |
| | | TTACGTACTAGATACG-3′ |
| 12 | TTR | 3′-aatgccatgatctatgc-5′-5′-TCTTGGTTACATGAAATCCC-3′-5′- |
| | | ttacgtactagatacg-3′ |
| 13 | ANGPLT3 | 3′-AATGCCATGATCTATGC-5′-5′-GGACATTGCCAGTAATCGCA-3′- |
| | | 5′-TTACGTACTAGATACG-3′ |
| 14 | ANGPLT3 | 3′-AATGCCATGATCTATGC-5′-5′-GGACATTGCCAGTAATCGCA-3′- |
| | | 5′-ttacgtactagatacg-3′ |
| 15 | ANGPLT3 | 3′-aatgccatgatctatgc-5′-5′-GGACATTGCCAGTAATCGCA-3′-5′- |
| | | TTACGTACTAGATACG-3′ |
| 16 | ANGPLT3 | 3′-aatgccatgatctatgc-5′-5′-GGACATTGCCAGTAATCGCA-3′-5′- |
| | | ttacgtactagatacg-3′ |
| 17 | Spinraza | 3′-AATGCCATGATCTATGC-5′-5′- |
| | | T₁C₁A₁C₁T₁T₁T₁C₁A₁T₁A₁A₁T₁G₁C₁T₁G₁G₁-3′-5′- |
| | | TTACGTACTAGATACG-3′ |
| 18 | Spinraza | 3′-AATGCCATGATCTATGC-5′-5′- |
| | | T₁C₁A₁C₁T₁T₁T₁C₁A₁T₁A₁A₁T₁G₁C₁T₁G₁G₁-3′-5′-ttacgtactagatacg-3′ |
| 19 | Spinraza | 3′-aatgccatgatctatgc-5′-5′-T₁C₁A₁C₁T₁T₁T₁C₁A₁T₁A₁A₁T₁G₁C₁T₁G₁G₁-3′- |
| | | 5′-TTACGTACTAGATACG-3′ |
| 20 | Spinraza | 3′-aatgccatgatctatgc-5′-5′-T₁C₁A₁C₁T₁T₁T₁C₁A₁T₁A₁A₁T₁G₁C₁T₁G₁G₁-3′- |
| | | 5′-ttacgtactagatacg-3′ |
| 21 | ApoL1 | 3′-AATGCCATGATCTATGC-5′-5′-TGCTCCGTTGGTGCTTGTTC-3′-5′- |
| | | TTACGTACTAGATACG-3′ |
| 22 | ApoL1 | 3′-AATGCCATGATCTATGC-5′-5′-TGCTCCGTTGGTGCTTGTTC-3′-5′- |
| | | ttacgtactagatacg-3′ |
| 23 | ApoL1 | 3′-aatgccatgatctatgc-5′-5′-TGCTCCGTTGGTGCTTGTTC-3′-5′- |
| | | TTACGTACTAGATACG-3′ |
| 24 | ApoL1 | 3′-aatgccatgatctatgc-5′-5′-TGCTCCGTTGGTGCTTGTTC-3′-5′- |
| | | ttacgtactagatacg-3′ |
| 25 | SOD1 | 3′-AATGCCATGATCTATGC-5′-5′-CAGGATACATTTCTACAGCT-3′- |
| | | 5′-TTACGTACTAGATACG-3′ |
| 26 | SOD1 | 3′-AATGCCATGATCTATGC-5′-5′-CAGGATACATTTCTACAGCT-3′- |
| | | 5′-ttacgtactagatacg-3′ |
| 27 | SOD1 | 3′-aatgccatgatctatgc-5′-5′-CAGGATACATTTCTACAGCT-3′-5′- |
| | | TTACGTACTAGATACG-3′ |
| 28 | SOD1 | 3′-aatgccatgatctatgc-5′-5′-CAGGATACATTTCTACAGCT-3′-5′- |
| | | ttacgtactagatacg-3′ |
| 29 | SOD1 | 3′-A₂A₂T₂C₂T₂G₂T₂G₂A₂T₂C₂C₂C₂T₂-5′-5′- |
| | | C₃A₃G₃G₃A₃T₃A₃C₃ATTTCTACAGCT-3′-5′- |
| | | T₂T₂A₂G₂A₂C₂A₂C₂T₂A₂G₂G₂G₂A₂-3′ |
| 30 | SOD1 | 3′-T₂G₂T₂G₂A₂T₂C₂C₂C₂T₂-5′-5′-C₃A₃G₃G₃A₃T₃A₃C₃ATTTCTACAGCT- |
| | | 3′-5′-A₂C₂A₂C₂T₂A₂G₂G₂G₂A₂-3′ |
| 31 | SOD1 | 3′-T₂G₂A₂T₂C₂C₂C₂T₂-5′-5′-C₃A₃G₃G₃A₃T₃A₃C₃ATTTCTACAGCT-3′-5′- |
| | | A₂C₂T₂A₂G₂G₂G₂A₂-3′ |
| 32 | SOD1 | 3′-A₂A₂T₂C₂T₂G₂T₂G₂A₂T₂C₂C₂C₂T₂-5′-5′- |
| | | C₃A₃G₃G₃A₃T₃A₃C₃ATTTCTACAGCT-3′-5′-T₂T₂A₂G₂a₁c₁a₁c₁t₁a₁g₁g₁g₁a₁-3′ |
| 33 | SOD1 | 3′-T₂G₂T₂G₂A₂T₂C₂C₂C₂T₂-5′-5′-C₃A₃G₃G₃A₃T₃A₃C₃ATTTCTACAGCT- |
| | | 3′-5′-a₁c₁a₁c₁t₁a₁g₁g₁g₁a₁-3′ |
| 34 | SOD1 | 3′-T₂G₂A₂T₂C₂C₂C₂T₂-5′-5′-C₃A₃G₃G₃A₃T₃A₃C₃ATTTCTACAGCT-3′-5′- |
| | | a₁c₁t₁a₁g₁g₁g₁a₁-3′ |
| 35 | APOC3 | 3′-A₂A₂T₂C₂T₂G₂T₂G₂A₂T₂C₂C₂C₂T₂-5′-5′- |
| | | G₃C₃T₃T₃C₃TTGTCCAGCTT₃T₃A₃T₃T₃-3′-5′- |
| | | T₂T₂A₂G₂A₂C₂A₂C₂T₂A₂G₂G₂G₂A₂-3′ |
| 36 | APOC3 | 3′-T₂G₂T₂G₂A₂T₂C₂C₂C₂T₂-5′-5′-G₃C₃T₃T₃C₃TTGTCCAGCTT₃T₃A₃T₃T₃- |
| | | 3′-5′-A₂C₂A₂C₂T₂A₂G₂G₂G₂A₂-3′ |
| 37 | APOC3 | 3′-T₂G₂A₂T₂C₂C₂C₂T₂-5′-5′-G₃C₃T₃T₃C₃TTGTCCAGCTT₃T₃A₃T₃T₃-3′-5′- |
| | | A₂C₂T₂A₂G₂G₂G₂A₂-3′ |
| 38 | APOC3 | 3′-A₂A₂T₂C₂T₂G₂T₂G₂A₂T₂C₂C₂C₂T₂-5′-5′- |
| | | G₃C₃T₃T₃C₃TTGTCCAGCTT₃T₃A₃T₃T₃-3′-5′-T₂T₂A₂G₂a₁c₁a₁c₁t₁a₁g₁g₁g₁a₁- |
| | | 3′ |
| 39 | APOC3 | 3′-T₂G₂T₂G₂A₂T₂C₂C₂C₂T₂-5′-5′-G₃C₃T₃T₃C₃TTGTCCAGCTT₃T₃A₃T₃T₃- |
| | | 3′-5′-a₁c₁a₁c₁t₁a₁g₁g₁g₁a₁-3′ |
| 40 | APOC3 | 3′-T₂G₂A₂T₂C₂C₂C₂T₂-5′-5′-G₃C₃T₃T₃C₃TTGTCCAGCTT₃T₃A₃T₃T₃-3′-5′- |
| | | a₁c₁t₁a₁g₁g₁g₁a₁-3′ |
| 41 | APOC3 | 3′-A₂A₂T₂C₂T₂G₂T₂G₂A₂T₂C₂C₂C₂T₂-5′-5′- |
| | | G₃C₃T₃T₃C₃T₃T₃GTCCAGCTTTATT-3′-5′- |
| | | T₂T₂A₂G₂A₂C₂A₂C₂T₂A₂G₂G₂G₂A₂-3′ |
| 42 | APOC3 | 3′-T₂G₂T₂G₂A₂T₂C₂C₂C₂T₂-5′-5′-G₃C₃T₃T₃C₃T₃T₃G₃TCCAGCTTTATT-3′- |
| | | 5′-A₂C₂A₂C₂T₂A₂G₂G₂G₂A₂-3′ |
| 43 | APOC3 | 3′-T₂G₂A₂T₂C₂C₂C₂T₂-5′-5′-G₃C₃T₃T₃C₃T₃T₃G₃TCCAGCTTTATT-3′-5′- |
| | | A₂C₂T₂A₂G₂G₂G₂A₂-3′ |
| 44 | APOC3 | 3′-A₂A₂T₂C₂T₂G₂T₂G₂A₂T₂C₂C₂C₂T₂-5′-5′- |
| | | G3C3T3T3C3T3T3G3TCCAGCTTTATT-3′-5′-T₂T₂A₂G₂a₁c₁a₁c₁t₁a₁g₁g₁g₁a₁-3′ |
| 45 | APOC3 | 3′-T₂G₂T₂G₂A₂T₂C₂C₂C₂T₂-5′-5′-G₃C₃T₃T₃C₃T₃T₃G₃TCCAGCTTTATT-3′- |
| | | 5′-a₁c₁a₁c₁t₁a₁g₁g₁g₁a₁-3′ |
| 46 | APOC3 | 3′-T₂G₂A₂T₂C₂C₂C₂T₂-5′-5′-G₃C₃T₃T₃C₃T₃T₃GTCCAGCTTTATT-3′-5′- |
| | | a₁c₁t₁a₁g₁g₁g₁a₁-3′ |
| 47 | APOC3 | 3′-T₂A₂C₂A₂G₂A₂T₂C₂T₂A₂C₂T₂G₂A₂-5′-5′- |
| | | G₃C₃T₃T₃C₃T₃T₃G₃TCCAGCTTTATT-3′-5′- |
| | | A₂T₂G₂T₂C₂T₂A₂G₂A₂T₂G₂A₂C₂T₂-3′ |
| 48 | APOC3 | 3′-T₂A₂C₂A₂G₂A₂T₂C₂T₂A₂C₂T₂G₂A₂-5′-5′- |
| | | G₃C₃T₃T₃C₃T₃T₃G₃TCCAGCTTTATT-3′-5′-A₂T₂G₂T₂C₂T₂A₂G₂A₂T₂-3′ |
| 49 | APOC3 | 3′-T₂A₂C₂A₂G₂A₂T₂C₂T₂A₂C₂T₂G₂A₂-5′-5′- |
| | | G₃C₃T₃T₃C₃T₃T₃G₃TCCAGCTTTATT-3′-5′-A₂T₂G₂T₂C₂T₂-3′ |
| 50 | APOC3 | 3′-G₂A₂T₂C₂T₂A₂C₂T₂G₂A₂-5′-5′-G₃C₃T₃T₃C₃T₃T₃G₃TCCAGCTTTATT-3′- |
| | | 5′-A₂T₂G₂T₂C₂T₂A₂G₂A₂T₂G₂A₂C₂T₂-3′ |
| 51 | APOC3 | 3′-C₂T₂A₂C₂T₂G₂A₂-5′-5′-G₃C₃T₃T₃C₃T₃T₃G₃TCCAGCTTTATT-3′-5′- |
| | | A₂T₂G₂T₂C₂T₂A₂G₂A₂T₂G₂A₂C₂T₂-3′ |
| 52 | MAPT | 3′-A₂A₂T₂C₂T₂G₂T₂G₂A₂T₂C₂C₂C₂T₂-5′-5′- |
| | | C₃C₃G₃T₃T₃T₃T₃CTTACCACCCT-3′-5′- |
| | | T₂T₂A₂G₂A₂C₂A₂C₂T₂A₂G₂G₂G₂A₂-3′ |
| 53 | MAPT | 3′-T₂G₂T₂G₂A₂T₂C₂C₂C₂T₂-5′-5′-C₃C₃G₃T₃T₃T₃T₃CTTACCACCCT-3′-5′- |
| | | A₂C₂A₂C₂T₂A₂G₂G₂G₂A₂-3′ |
| 54 | MAPT | 3′-T₂G₂A₂T₂C₂C₂C₂T₂-5′-5′-C₃C₃G₃T₃T₃T₃T₃CTTACCACCCT-3′-5′- |
| | | A₂C₂T₂A₂G₂G₂G₂A₂-3′ |
| 55 | MAPT | 3′-A₂A₂T₂C₂T₂G₂T₂G₂A₂T₂C₂C₂C₂T₂-5′-5′- |
| | | C₃C₃G₃T₃T₃T₃T₃CTTACCACCCT-3′-5′-T₂T₂A₂G₂a₁c₁a₁c₁t₁a₁g₁g₁g₁a₁-3′ |
| 56 | MAPT | 3′-T₂G₂T₂G₂A₂T₂C₂C₂C₂T₂-5′-5′-C₃C₃G₃T₃T₃T₃T₃CTTACCACCCT-3′-5′- |
| | | a₁c₁a₁c₁t₁a₁g₁g₁g₁a₁-3′ |
| 57 | MAPT | 3′-T₂G₂A₂T₂C₂C₂C₂T₂-5′-5′-C₃C₃G₃T₃T₃T₃T₃CTTACCACCCT-3′-5′- |
| | | a₁c₁t₁a₁g₁g₁g₁a₁-3′ |

Uppercase G/C/A/T- DNA with phosphorothioate linkage; lowercase a/t/c/g-RNA with phosphodiester linkage; G₁/C₁/A₁/U₁-2′OME or 2′ MOE ribonucleotide; A₂/T₂/C₂/G₂-DNA with phosphodiester linkage; lowercase a₁/t₁/c₁/g₁-RNA with phosphodiester linkage; G₃/C₃/A₃/U₃-2′ MOE ribonucleotide with phosphorothioate linkage

### Splicing Oligonucleotides.

In embodiments, the invention provides a circular prodrug nucleic acid (CPN) comprising a functional domain comprising a splicing oligonucleotide. In embodiments, the oligonucleotide of the functional domain is modified.

As used herein, the term “splicing oligonucleotide” refers to an antisense oligonucleotide for modulating splicing. For splice modulation, the antisense oligonucleotide binds to the target RNA and modulates splicing, thereby the expression of a protein. The circular structure allows reduced protein binding, reduced polyanionic characteristic, and lack of accessibility to the ends which permits endosomal escape and mitigates interaction with pattern recognition receptors.

In embodiments, the oligonucleotide for modulating splicing is a snRNA. The terms “snRNA” and “small nuclear RNA” are interchangeable and refer to a class of small RNAs involved in a variety of processes including RNA splicing and regulation of transcription factors. The subclass of small nucleolar RNAs (snoRNAs) is also included. The term is also intended to include artificial snRNAs, such as antisense derivatives of snRNAs. The design of such snRNA is within the skill of ordinary artisans.

In embodiments, the oligonucleotide of the functional domain is as described in WO 2021/055011, which is incorporated herein by reference in its entirety. Specifically, in embodiments, the oligonucleotide of the functional domain comprises an oligonucleotide comprising 14 to 30 linked nucleotides complementary to a target pre-mRNA comprising a retained intron, wherein the antisense oligonucleotide comprises 1 to 3 DNA regions each region independently comprising from 2 to 5 consecutive deoxyribonucleotides and the remaining nucleotides are 2′-substituted, non-ionic or constrained sugar nucleotides, or combinations thereof. In embodiments, the 2′-substituted nucleotides are selected from 2′ O-methyl ribonucleosides or 2′-methoxyethyl ribonucleosides (MOE).

A CPN having a functional domain comprising an oligonucleotide that modulates splicing is useful for selecting a first mRNA transcript in a gene comprising at least two mRNA transcripts, wherein the oligonucleotide comprises at least 12 contiguous nucleobases complementary to an equal length portion of a target pre-mRNA; wherein the oligonucleotide targets a splice site of the pre-mRNA for a second mRNA transcript thereby blocking the splice site for the second mRNA transcript and directing splicing of the pre-mRNA to the first mRNA transcript.

In embodiments, the splicing oligonucleotide comprises 1 region comprising from 2 to 5 consecutive deoxyribonucleotides, and the remaining nucleotides are 2′-substituted, non-ionic or constrained sugar nucleotides, or combinations thereof. In embodiments, the splicing oligonucleotide comprises 2 regions independently comprising from 2 to 5 consecutive deoxyribonucleotides, and the remaining nucleotides are 2′-substituted, non-ionic, or constrained sugar nucleotides, or combinations thereof. In embodiments, the splicing comprises 3 regions independently comprising from 2 to 5 consecutive deoxyribonucleotides, and the remaining nucleotides are 2′-substituted, non-ionic, or constrained sugar nucleotides, or combinations thereof. In some embodiments, the consecutive deoxyribonucleotides are 2-4 nucleotides in length. In some embodiments, the consecutive deoxyribonucleotides are 4 nucleotides in length.

In embodiments, the region of consecutive deoxyribonucleotides of the splicing oligonucleotide are at the 5′ end of the antisense oligonucleotide, at the 3′ end of the splicing oligonucleotide, or flanked by the 2′-substituted, non-ionic, or constrained sugar nucleotides, or combinations thereof. In embodiments, the consecutive deoxyribonucleotides are at the 5′ end of the splicing oligonucleotide. In embodiments, the consecutive deoxyribonucleotides are at the 3′ end of the splicing oligonucleotide. In embodiments, the consecutive deoxyribonucleotides are flanked by the 2′-substituted, non-ionic, or constrained sugar nucleotides, or combinations thereof.

In embodiments, the splicing oligonucleotide of the functional domain comprises 14 to 30 linked nucleotides having at least 12 contiguous nucleobases complementary to an equal length portion of a target pre-mRNA comprising a retained intron, wherein the antisense oligonucleotide comprises 1 to 3 DNA regions each region independently comprising from 2 to 5 consecutive deoxyribonucleotides and the remaining nucleotides are 2′-substituted, non-ionic or constrained sugar nucleotides, or combinations thereof.

In embodiments, the 2′-substituted nucleotides are as described herein. In embodiments, the 2′-substituted nucleotides are selected from 2′ O-methyl ribonucleotides or 2′-MOE.

In embodiments, the oligonucleotide of the functional domain comprises 1 region comprising from 2 to 5 consecutive deoxyribonucleotides. In embodiments, the consecutive deoxyribonucleotides are at the 5′ end of the splicing oligonucleotide, at the 3′ end of the antisense oligonucleotide, flanked by at the 2′-substituted, non-ionic, or constrained sugar nucleotides, or combinations thereof. In embodiments, the consecutive deoxyribonucleotides are at the 5′ end of the splicing oligonucleotide. In embodiments, the consecutive deoxyribonucleotides are at the 3′ end of the splicing oligonucleotide.

In embodiments, the consecutive deoxyribonucleotides are 2-4 nucleotides in length. In embodiments, the consecutive deoxyribonucleotides are 4 nucleotides in length.

In embodiments, an exon flanks the 5′ splice site of the retained intron. In embodiments, an exon flanks the 3′ splice site of the retained intron. In embodiments, an exon flanks the 5′ splice site of the retained intron and an exon flanks the 3′ splice site of the retained intron.

### ADAR

In embodiments, the invention provides a circular prodrug nucleic acid (CPN) comprising a functional domain comprising an antisense oligonucleotide of an adenosine deaminase acting on RNA (ADAR) system. ADARs are a group of enzymes that catalyze the conversion of adenosines (A's) to inosines (I's) in a process known as RNA editing. Though ADARs can act on different types of RNA, editing events in coding regions of mRNA are of particular interest as I's base pair like guanosines (G's). Thus, every A-to-I change catalyzed by ADAR is read as an A-to-G change during translation, potentially altering protein sequence and function. This ability to re-code makes ADAR an attractive therapeutic tool to correct genetic mutations within mRNA.

The recognition domain recognizes and binds to specific double-stranded RNA (dsRNA) and the catalytic domain converts adenosine to inosine in the target dsRNA through deamination. In RNA, inosine functions similarly to guanosine for translation and replication, for example because of its similarity to guanosine inosine will hybridize and/or bind to cytosine whereas prior to the deamination of the adenosine to inosine, the corresponding nucleotide pair would be thymine. Additionally, inosine is most often found to mimic guanosine for translational purposes. Thus, conversion of adenosine to inosine in an mRNA can result in a codon change that may lead to changes to the encoded protein and its functions. There are three known ADAR proteins expressed in humans, ADAR1, ADAR2, and ADAR3. ADAR1 and ADAR2 are expressed throughout the body whereas ADAR3 is expressed only in the brain.

ADAR proteins are naturally expressed proteins in various cells, tissues, organs and/or organism. It has been reported that some ADAR proteins, e.g., ADAR1 and ADAR2, can edit adenosine through deamination, converting adenosine to inosine which can provide a number of functions including being read as or similar to G during translation. Mechanism of ADAR-mediated mRNA editing (e.g., deamination) has been reported. For example, ADAR proteins are reported to catalyze conversion of adenosine to inosine on double-stranded RNA substrates with mismatches. As appreciated by those skilled in the art, inosine can be recognized as guanosine by cellular translation and/or splicing machinery. ADAR can thus be used for functional adenosine to guanosine editing of nucleic acids, e.g., pre-mRNA and mRNA substrates.

Without being bound by any theory or hypothesis, ADAR-recruiting RNA (“arRNA”) recognize and bind to target nucleic acids comprising adenosine for ADAR-mediated editing of target adenosine in target nucleic acids, e.g. RNA. The arRNA acts through hybridizing to its target RNA in a sequence-specific fashion to form a double-stranded RNA, which recruits an Adenosine Deaminase Acting on RNA (ADAR) to deaminate a target adenosine in the target RNA.

ADAR-mediated RNA-editing can offer several advantages over DNA-editing, e.g., delivery is simplified as expression of recombinant proteins like Cas9 is not required. Both ADAR1 and ADAR2 are endogenous enzymes, so cellular delivery of arRNAs alone can be sufficient for editing. Off-target effects, if any, are transient as changes are not made to genomic DNA. Additionally, ADAR-mediated editing can be used in post-mitotic cells and it does not require an HDR-template for repair. Three vertebrate ADAR genes have been reported with common functional domains (Nishikura Nat Rev Mol Cell Biol. 2016 February; 17(2): 83-96; Nishikura Annu Rev Biochem. 2010; 79:321-349; Thomas and Beal Bioessays. 2017 April; 39(4)). All 3 ADARs contain a dsRNA-binding domains (dsRBD), which can contact dsRNA substrates. Some ADAR1 also contains Z-DNA-binding domains. ADAR1 has been reported to express significantly in brain, lung, kidney, liver, and heart, etc., and may occur in two isoforms. In some embodiments, isoform p150 can be induced by interferon while isoform p110 can be constitutively expressed. In some embodiments, it can be beneficial to utilize p110 as it is reported to be ubiquitously and constitutively expressed. ADAR2 can be highly expressed, e.g., in the brain and lungs, and is reported to be exclusively localized to the nucleus. ADAR3 is reported to be catalytically inactive and expressed only in the brain. Potential differences in tissue expression can be taken into consideration when choosing a therapeutic target.

Use of oligonucleotides for RNA editing by ADAR has been reported. Among other things, the present disclosure recognizes that previously reported technologies generally suffer one or more disadvantages, such as low stability (e.g., oligonucleotides with natural RNA sugars), low editing efficiency, low editing specificity (e.g., a number of As are edited in a portion of a target nucleic acid substantially complementary to an oligonucleotide), specific structures in oligonucleotides for ADAR recognition/recruitment, exogenous proteins (e.g., those engineered to recognize oligonucleotides with specific structures and/or duplexes thereof (e.g., with target nucleic acids) for editing), etc.

In embodiments, the provided CPN-arRNAs can direct a correction of a guanosine (G) to adenosine (A) mutation in a target sequence, or a product thereof. In some embodiments, a correction of a G to A mutation is or comprises conversion of A to inosine (I), which can be read as G during translation or other biological processes. In embodiments, the provided CPN-arRNAs can direct a correction of a G to A mutation in a target sequence or a product thereof via ADAR-mediated deamination. In embodiments, the provided CPN-arRNAs can direct a correction of a G to A mutation in a target sequence or a product thereof via ADAR-mediated deamination by recruiting an endogenous ADAR (e.g., in a target cell) and facilitating the ADAR-mediated deamination. Regardless, however, the present disclosure is not limited to any particular mechanism. In some embodiments, the present disclosure provides CPN-arRNAs, compositions, methods, etc., capable of operating via double-stranded RNA interference, single-stranded RNA interference, RNase H-mediated knock-down, steric hindrance of translation, ADAR-mediated deamination or a combination of two or more such mechanisms.

In embodiments, the CPN-arRNA can hybridize to a target RNA sequence nucleic acid in any stage of RNA processing, including but not limited to a pre-mRNA or a mature mRNA. In some embodiments, the CPN-arRNA can hybridize to any element of oligonucleotide nucleic acid or its complement, including but not limited to: a promoter region, an enhancer region, a transcriptional stop region, a translational start signal, a translation stop signal, a coding region, a non-coding region, an exon, an intron, an intron/exon or exon/intron junction, the 5′ UTR, or the 3′ UTR. In some embodiments, the CPN-arRNA hybridizes to two or more variants of transcripts derived from a sense strand of a target site (e.g., a target sequence).

In some embodiments, a CPN-arRNA or composition is characterized in that, when it is contacted with a target nucleic acid comprising a target adenosine in a system (e.g., an ADAR-mediated deamination system), modification of the target adenosine (e.g., deamination of the target A) is improved relative to that observed under reference conditions (e.g., selected from the group consisting of absence of the composition, presence of a reference oligonucleotide or composition, and combinations thereof).

As appreciated by those skilled in the art, structural features of the present disclosure, such as nucleobase modification, sugar modifications, internucleotidic linkage modifications, linkage phosphorus stereochemistry, etc., and combinations thereof may be utilized with various suitable base sequences to provide CPN-arRNAs and compositions with desired properties and/or activities. For example, CPN-arRNAs for adenosine modification (e.g., conversion to I in the presence of ADAR proteins) typically have sequences that are sufficiently complementary to sequences of target nucleic acids that comprise target adenosines. In many embodiments, e.g., for targeting G to A mutations, CPN-arRNAs may selectively target one and only one target adenosine for modification, e.g., by ADAR to convert into I.

Base sequences of the CPN-arRNA, as appreciated by those skilled in the art, typically have sufficient lengths and complementarity to their target nucleic acids, e.g., RNA transcripts (e.g., pre-mRNA, mature mRNA, etc.) for, e.g., site-directed editing of target adenosines. In some embodiments, the arRNA of the CPN-arRNA is complementary to a portion of a target RNA sequence comprising a target adenosine (as appreciated by those skilled in the art, in many instances target nucleic acids are longer than the arRNA of the CPN-arRNAs of the present disclosure, and complementarity may be properly assessed based on the shorter of the two).

As appreciated by those skilled in the art, the CPN-arRNAs of the invention may be utilized to improve oligonucleotides in prior technologies (e.g., those described in WO2016097212, WO2017220751, WO2018041973, WO2018134301A1, WO2021071858, WO2022091100, oligonucleotides and oligonucleotide compositions of each of which are independently incorporated by reference). In some embodiments, the present disclosure provides improvements of prior technologies by applying the CPN-arRNAs described herein to prior reported oligonucleotide base sequences. In some embodiments, the present disclosure provides CPN-arRNA compositions of previously reported oligonucleotides that may be useful for adenosine editing. In some embodiments, the present disclosure provides improvements of previously reported adenosine editing oligonucleotide compositions by performing such editing using CPN-arRNA compositions.

In some embodiments, a CPN-arRNA can form a dsRNA structure with a target mRNA through base pairing. In some embodiments, formed dsRNA structures (optionally with secondary mismatches) contain bulges that promote ADAR binding and therefore, can facilitate ADAR-mediated editing (e.g., deamination of a target adenosine). Various technologies may be utilized to assess/characterize CPN-arRNAs in accordance with the present disclosure.

In some embodiments, the oligonucleotide of the functional domain is an antisense oligonucleotide that recruit endogenous ADAR (adenosine deaminase acting on RNA) enzymes to edit endogenous transcripts.

The oligonucleotide of an ADAR system itself comprises one or more domains. One domain, known as the complementary domain, comprises a region of consecutive nucleotides that are complementary to the target RNA. In embodiments, the complementary domain is from about 15 to about 120 nucleotides in length. In embodiments, the complementary domain is from about 17 to about 60 nucleotides in length. Another domain, known as the recruiting domain, comprises a region of the oligonucleotide that recruits ADAR enzymes.

In embodiments where the functional domain of the CPN is an antisense oligonucleotide of an ADAR system, the length of the first nucleic acid molecule and the second nucleic acid molecule can be longer. For example, the first nucleic acid molecule and the second nucleic acid molecule each independently comprises an oligonucleotide between 6 and 50 nucleotides in length. In embodiments, the first nucleic acid molecule and the second nucleic acid molecule of the circularizing domain each independently comprises an oligonucleotide between 6 and 25 nucleotides in length. In embodiments, the first nucleic acid molecule and the second nucleic acid molecule independently comprises an oligonucleotide between 6 and 12 nucleotides in length. In embodiments, the first nucleic acid molecule and the second nucleic acid molecule independently comprises an oligonucleotide between 6 and 10 nucleotides in length. In embodiments, the first nucleic acid molecule and the second nucleic acid molecule independently comprises an oligonucleotide between 6 and 8 nucleotides in length.

### CRISPR

In embodiments, the invention provides a circular prodrug nucleic acid (CPN) comprising a functional domain comprising an antisense oligonucleotide (e.g., guide RNA) of a CRISPR-based system.

The CRISPR/Cas system (“CRISPR system”) utilizes a short RNA molecule (e.g., a guideRNA) to recognize a specific DNA target and to recruit a Cas enzyme to the specific DNA target.

In general, a CRISPR system refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (guideRNA), or other sequences and transcripts from a CRISPR locus. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes.

In general, a CRISPR system is characterized by elements that promote the formation of a CRISPR complex at the site of a target sequence. In the context of formation of a CRISPR complex, “target sequence” refers to a sequence to which a guideRNA is designed to have complementarity, where hybridization between a target sequence and a guideRNA promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In some embodiments, a target sequence is located in the nucleus or cytoplasm of a cell. In some embodiments, the target sequence may be within an organelle of a eukaryotic cell, for example, mitochondrion or chloroplast. In an aspect of the invention the recombination is homologous recombination.

Typically, in the context of an endogenous CRISPR system, formation of a CRISPR complex (comprising a guideRNA hybridized to a target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guideRNA. In some embodiments, the tracr sequence has sufficient complementarity to a tracr mate sequence to hybridize and participate in formation of a CRISPR complex.

As with the target sequence, a complete complementarity is not needed, provided there is sufficient hybridization and/or binding to be functional. In some embodiments, the tracr sequence has at least 50%, 60%, 70%, 80%, 90%, 95% or 99% of sequence complementarity along the length of the tracr mate sequence when aligned.

In some embodiments, one or more vectors driving expression of one or more elements of a CRISPR system are introduced into a host cell such that expression of the elements of the CRISPR system direct formation of a CRISPR complex at one or more target sites. For example, a Cas enzyme, a guideRNA linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. CRISPR system elements that are combined in a single vector may be arranged in any suitable orientation, such as one element located 5—with respect to (“upstream” of) or 3′ with respect to (“downstream” of) a second element. The coding sequence of one element may be located on the same or opposite strand of the coding “sequence” of a second element, and oriented in the same or opposite direction. In some embodiments, a single promoter drives expression of a transcript encoding a CrISPR. enzyme and one or more of the guideRNA, tracr mate sequence (optionally operably linked to the guide sequence), and a tracr sequence embedded within one or more intron sequences (e.g. each in a different intron, two or more in at least one in iron, or ail in a single intron). In some embodiments, the CRISPR enzyme, guideRNA, tracr mate sequence, and tracr sequence are operably linked to and expressed from the same promoter.

Non-limiting examples of Cas proteins include Cas1, Cas1 B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof or modified versions thereof. In some embodiments, the unmodified CRISPR enzyme has DNA cleavage activity, such as Cas9. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence.

In some embodiments, a vector encodes a CRISPR enzyme that is mutated to with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A. In aspects of the invention, nickases may be used for genome editing via homologous recombination.

In general, a guideRNA is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

In some embodiments, a guideRNA is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. The ability of a guide sequence to direct sequence-specific binding of a CRISPR complex to a target sequence may be assessed by any suitable assay. For example, the components of a CRISPR system sufficient to form a CRISPR complex, including the guideRNA to be tested, may be provided to a host cell having the corresponding target sequence, such, as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guideRNA to be tested and a control guideRNA different from the test guideRNA, and comparing binding or rate of cleavage at the target sequence between the test and control guide sequence reactions. Other assays are possible, and will occur to those skilled in the art. A guideRNA may be selected to target any target sequence. In some embodiments, the target sequence is a sequence within a genome of a cell.

The terms “gRNA” and “guideRNA” are interchangeable and refer to a specific RNA sequence that recognizes the target DNA or RNA region of interest and directs an endonuclease to that location for gene editing.

The gRNA is usually made up of two parts: crispr RNA (crRNA), a 17-30 nucleotide sequence complementary to the target DNA, and a tracr RNA, which serves as a binding scaffold for the Cas nuclease. Any suitable engineered gRNA, or crRNA and tracrRNA, can be employed as long as it is effective for recognizing a target DNA or RNA. The design of such gRNA, or crRNA and tracrRNA is within the skill of ordinary artisans.

The present invention provides a CPN comprising a guideRNA for a CRISPR-based system as the functional domain (“CPN-gRNA”). CPN-gRNAs according to the invention are described herein.

The Cpn-gRNAs according to the invention can be used in a CRISPR system as known to one skilled in the art. For example, suitable CRISPR systems can include, but are not limited to WO 2013/176772, WO 2013/188638, WO 2014/018423, WO 2014/022702, WO 2014/093661, WO 2014/093622, WO 2017/004279, WO 2017/189308, WO 2018/119354, WO 2018/208998, WO 2019/089804, WO 2019/089910, WO 2019/089808, WO 2019/089796, WO 2019/089820, and WO 2021/087394, the contents of each of which are herein incorporated by reference in their entireties.

The oligonucleotide of the functional domain is an antisense oligonucleotide that functions as a guideRNA for a CRISPR-based system. The terms “sgRNA” and “guideRNA” are interchangeable and refer to a specific RNA sequence that recognizes the target DNA or RNA region of interest and directs the endonuclease there for editing. The gRNA is usually made up of two parts: crispr RNA (crRNA), a 17-30 nucleotide sequence complementary to the target DNA, and a tracr RNA, which serves as a binding scaffold for the Cas nuclease.

Any suitable engineered sgRNA, or crRNA and tracrRNA, can be employed as long as it is effective for recognizing a target DNA or RNA. The design of such sgRNA, or crRNA and tracrRNA is within the skill of ordinary artisans.

In embodiments where the functional domain of the CPN is an antisense oligonucleotide of a CRISPR-based system, the length of the first nucleic acid molecule and the second nucleic acid molecule can be longer. For example, the first nucleic acid molecule and the second nucleic acid molecule each independently comprises an oligonucleotide between 6 and 50 nucleotides in length. In embodiments, the first nucleic acid molecule and the second nucleic acid molecule of the circularizing domain each independently comprises an oligonucleotide between 6 and 25 nucleotides in length. In embodiments, the first nucleic acid molecule and the second nucleic acid molecule independently comprises an oligonucleotide between 6 and 12 nucleotides in length. In embodiments, the first nucleic acid molecule and the second nucleic acid molecule independently comprises an oligonucleotide between 6 and 10 nucleotides in length. In embodiments, the first nucleic acid molecule and the second nucleic acid molecule independently comprises an oligonucleotide between 6 and 8 nucleotides in length.

mRNA

In embodiments, the invention provides a circular prodrug nucleic acid (CPN) comprising a functional domain comprising a nucleic acid molecule, specifically a polynucleotide, primary constructs and/or mRNA which encode one or more polypeptides of interest or fragments thereof and which retains sufficient structural and/or chemical features to allow the polypeptide of interest encoded therein to be translated. Such polypeptide of interest may include, but is not limited to, whole polypeptides, a plurality of polypeptides or fragments of polypeptides, which independently may be encoded by one or more regions or parts or the whole of a polynucleotide. As used herein, the term “polypeptides of interest” refer to any polypeptide which is selected to be encoded within, or whose function is affected by, the polynucleotides of the present invention.

In preferred embodiments, the nucleic acid molecule is a messenger RNA (mRNA). As used herein, the term “messenger RNA” (mRNA) refers to any polynucleotide which encodes a polypeptide of interest and which is capable of being translated to produce the encoded polypeptide of interest in vitro, in vivo, in situ or ex vivo.

In embodiments, the basic components of an mRNA molecule include at least a coding region, a 5′UTR, a 3′UTR, a 5′ cap and a poly-A tail.

In embodiments, the mRNA molecules comprise one or more structural and/or chemical modifications or alterations which impart useful properties to the polynucleotide including, in some embodiments, the lack of a substantial induction of the innate immune response of a cell into which the polynucleotide is introduced. As used herein, a “structural” feature or modification is one in which two or more linked nucleotides are inserted, deleted, duplicated, inverted or randomized in a polynucleotide without significant chemical modification to the nucleotides themselves. Structural modifications will result in a different sequence of nucleotides. For example, the polynucleotide “ATCG” may be chemically modified to “AT-5meC-G”. The same polynucleotide may be structurally modified from “ATCG” to “ATCCCG”. Here, the dinucleotide “CC” has been inserted, resulting in a structural modification to the polynucleotide.

In embodiments, the mRNA functional domain comprises a first region of linked nucleotides that is flanked by a first flanking region and a second flaking region. This first region may include, but is not limited to, the encoded polypeptide of interest. The polypeptide of interest may comprise at its 5′ terminus one or more signal sequences encoded by a signal sequence region. The first flanking region may comprise a region of linked nucleotides comprising one or more complete or incomplete 5′ UTRs sequences. The first flanking region may also comprise a 5′ terminal cap. The second flanking region may comprise a region of linked nucleotides comprising one or more complete or incomplete 3′ UTRs. The second flanking region may also comprise a 3′ tailing sequence.

In embodiments, a first operation region bridges the 5′ terminus of the first region and the first flanking region. This operational region comprises a Start codon. The operational region may alternatively comprise any translation initiation sequence or signal including a Start codon.

In embodiments, a second operation region Bridges the 3′ terminus of the first region and the second flanking region. This second operational region comprises a Stop codon. The second operational region may alternatively comprise any translation initiation sequence or signal including a Stop codon. In embodiments, multiple serial stop codons may also be used.

In embodiments, the shortest length of the first region of the primary construct of the present invention can be the length of a nucleic acid sequence that is sufficient to encode for a dipeptide, a tripeptide, a tetrapeptide, a pentapeptide, a hexapeptide, a heptapeptide, an octapeptide, a nonapeptide, or a decapeptide. In another embodiment, the length may be sufficient to encode a peptide of 2-30 amino acids, e.g., 5-30, 10-30, 2-25, 5-25, 10-25, or 10-20 amino acids. The length may be sufficient to encode for a peptide of at least 11, 12, 13, 14, 15, 17, 20, 25 or 30 amino acids, or a peptide that is no longer than 40 amino acids, e.g., no longer than 35, 30, 25, 20, 17, 15, 14, 13, 12, 11 or 10 amino acids.

Generally, the length of the first region encoding the polypeptide of interest of the present invention is greater than about 30 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000, 4,000, 5,000, 6,000, 7,000, 8,000, 9,000, 10,000, 20,000, 30,000, 40,000, 50,000, 60,000, 70,000, 80,000, 90,000 or up to and including 100,000 nucleotides). As used herein, the “first region” may be referred to as a “coding region” or “region encoding” or simply the “first region.”

In some embodiments, mRNA includes from about 30 to about 100,000 nucleotides (e.g., from 30 to 50, from 30 to 100, from 30 to 250, from 30 to 500, from 30 to 1,000, from 30 to 1,500, from 30 to 3,000, from 30 to 5,000, from 30 to 7,000, from 30 to 10,000, from 30 to 25,000, from 30 to 50,000, from 30 to 70,000, from 100 to 250, from 100 to 500, from 100 to 1,000, from 100 to 1,500, from 100 to 3,000, from 100 to 5,000, from 100 to 7,000, from 100 to 10,000, from 100 to 25,000, from 100 to 50,000, from 100 to 70,000, from 100 to 100,000, from 500 to 1,000, from 500 to 1,500, from 500 to 2,000, from 500 to 3,000, from 500 to 5,000, from 500 to 7,000, from 500 to 10,000, from 500 to 25,000, from 500 to 50,000, from 500 to 70,000, from 500 to 100,000, from 1,000 to 1,500, from 1,000 to 2,000, from 1,000 to 3,000, from 1,000 to 5,000, from 1,000 to 7,000, from 1,000 to 10,000, from 1,000 to 25,000, from 1,000 to 50,000, from 1,000 to 70,000, from 1,000 to 100,000, from 1,500 to 3,000, from 1,500 to 5,000, from 1,500 to 7,000, from 1,500 to 10,000, from 1,500 to 25,000, from 1,500 to 50,000, from 1,500 to 70,000, from 1,500 to 100,000, from 2,000 to 3,000, from 2,000 to 5,000, from 2,000 to 7,000, from 2,000 to 10,000, from 2,000 to 25,000, from 2,000 to 50,000, from 2,000 to 70,000, and from 2,000 to 100,000).

In embodiments, the first and second flanking regions may range independently from 15-1,000 nucleotides in length (e.g., greater than 30, 40, 45, 50, 55, 60, 70, 80, 90, 100, 120, 140, 160, 180, 200, 250, 300, 350, 400, 450, 500, 600, 700, 800, and 900 nucleotides or at least 30, 40, 45, 50, 55, 60, 70, 80, 90, 100, 120, 140, 160, 180, 200, 250, 300, 350, 400, 450, 500, 600, 700, 800, 900, and 1,000 nucleotides).

In embodiments, the tailing sequence may range from absent to 500 nucleotides in length (e.g., at least 60, 70, 80, 90, 120, 140, 160, 180, 200, 250, 300, 350, 400, 450, or 500 nucleotides). Where the tailing region is a poly A tail, the length may be determined in units of or as a function of polyA Binding Protein binding. In this embodiment, the poly A tail is long enough to bind at least 4 monomers of PolyA Binding Protein. PolyA Binding Protein monomers bind to stretches of approximately 38 nucleotides. As such, it has been observed that poly A tails of about 80 nucleotides and 160 nucleotides are functional.

In embodiments, the capping region may comprise a single cap or a series of nucleotides forming the cap. In this embodiment the capping region may be from 1 to 10, e.g., 2-9, 3-8, 4-7, 1-5, 5-10, or at least 2, or 10 or fewer nucleotides in length. In some embodiments, the cap is absent.

In embodiments, the first and second operational regions may range from 3 to 40, e.g., 5-30, 10-20, 15, or at least 4, or 30 or fewer nucleotides in length and may comprise, in addition to a Start and/or Stop codon, one or more signal and/or restriction sequences.

In embodiments, the mRNA functional domain may be designed to encode polypeptides of interest selected from any of several target categories including, but not limited to, biologics, antibodies, vaccines, therapeutic proteins or peptides, cell penetrating peptides, secreted proteins, plasma membrane proteins, cytoplasmic or cytoskeletal proteins, intracellular membrane bound proteins, nuclear proteins, proteins associated with human disease, targeting moieties or those proteins encoded by the human genome for which no therapeutic indication has been identified but which nonetheless have utility in areas of research and discovery. In embodiments, the mRNA functional domain may encode variant polypeptides which have a certain identity with a reference polypeptide sequence. As used herein, a “reference polypeptide sequence” refers to a starting polypeptide sequence. Reference sequences may be wild type sequences or any sequence to which reference is made in the design of another sequence. The polypeptide variant may have the same or a similar activity as the reference polypeptide. Alternatively, the variant may have an altered activity (e.g., increased or decreased) relative to a reference polypeptide. Generally, variants of a particular polynucleotide or polypeptide of the invention will have at least about 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art.

In embodiments where the functional domain of the CPN is an mRNA, the length of the first nucleic acid molecule and the second nucleic acid molecule can be longer as compared to a CPN wherein the functional domain is a gene modulating oligonucleotide. For example, the first nucleic acid molecule and the second nucleic acid molecule each independently comprises an oligonucleotide between 6 and 50 nucleotides in length. In embodiments, the first nucleic acid molecule and the second nucleic acid molecule of the circularizing domain each independently comprises an oligonucleotide between 6 and 25 nucleotides in length. In embodiments, the first nucleic acid molecule and the second nucleic acid molecule independently comprises an oligonucleotide between 6 and 12 nucleotides in length. In embodiments, the first nucleic acid molecule and the second nucleic acid molecule independently comprises an oligonucleotide between 6 and 10 nucleotides in length. In embodiments, the first nucleic acid molecule and the second nucleic acid molecule independently comprises an oligonucleotide between 6 and 8 nucleotides in length.

### Immunostimulatory Oligonucleotides

In embodiments, the invention provides a circular prodrug nucleic acid (CPN) comprising a functional domain comprising an immunostimulatory oligonucleotide.

In embodiments, the immunostimulatory oligonucleotide is capable of inducing an interferon response in a vertebrate cell.

In embodiments, the nucleotide sequence of the immunostimulatory oligonucleotide is not complementary to and does not bind to another nucleotide sequence, for example, a target RNA. In this embodiment, the nucleotide sequence of the immunostimulatory oligonucleotide is not, for example, an antisense oligonucleotide and does not have antisense activity.

In embodiments, the immunostimulatory oligonucleotide is between 11 and 40 nucleotides in length. In embodiments, immunostimulatory oligonucleotide is between 15 and 28 nucleotides in length. In embodiments, immunostimulatory oligonucleotide is between 17 and 25 nucleotides in length.

In embodiments, the internucleotide linkages of the immunostimulatory oligonucleotide are phosphorothioate, phosphodiester, or combinations thereof.

In embodiments, the internucleotide linkages of the immunostimulatory oligonucleotide are phosphorothioate internucleotide linkages.

In embodiments, the internucleotide linkages of the immunostimulatory oligonucleotide are phosphodiester.

In embodiments, the internucleotide linkages of the immunostimulatory oligonucleotide are a combination of phosphorothioate and phosphodiester internucleotide linkages.

Immunostimulatory oligonucleotides include, but are not limited to, oligonucleotides that are Pathogen-associated molecular patterns (PAMPS) for pattern recognition receptors (PRRs).

Immunostimulatory oligonucleotides include, but are not limited to, oligonucleotides that induce immunostimulation through endosomal toll-like receptors, RIG like receptors, STING, cGAS and inflammasomes. Toll-like receptors (TLRs) are pattern recognition receptors (PRRs) which play a crucial in the initiation of innate immune response by detecting potential harmful pathogens. Each TLR has a broad range of specificities, for example, TLR1, 2, 4 and 6 recognize bacterial lipids, TLR3, 7 and 8 recognize viral RNA, TLR9 recognizes bacterial DNA comprising a CG motif, and TLR5 and 10 recognize bacterial or parasite proteins. The design of such immunostimulatory oligonucleotides is within the skill of ordinary artisans.

### Immune Antagonist

In embodiments, the invention provides a circular prodrug nucleic acid (CPN) comprising a functional domain comprising an immune antagonist oligonucleotide.

In embodiments, the immune antagonist oligonucleotide is capable of blocking an interferon response in a vertebrate cell.

In embodiments, the nucleotide sequence of the immune antagonist oligonucleotide is not complementary to and does not bind to another nucleotide sequence, for example, a target RNA. In this embodiment, the nucleotide sequence of the immune antagonist oligonucleotide is not, for example, an antisense oligonucleotide and does not have antisense activity.

In embodiments, the immune antagonist oligonucleotide is between 11 and 30 nucleotides in length. In embodiments, immune antagonist oligonucleotide is between 15 and 28 nucleotides in length. In embodiments, immune antagonist oligonucleotide is between 17 and 25 nucleotides in length.

In embodiments, the internucleotide linkages of the immune antagonist oligonucleotide are phosphorothioate, phosphodiester, or combinations thereof.

In embodiments, the internucleotide linkages of the immune antagonist oligonucleotide are phosphorothioate internucleotide linkages.

In embodiments, the internucleotide linkages of the immune antagonist oligonucleotide are phosphodiester.

In embodiments, the internucleotide linkages of the immune antagonist oligonucleotide are a combination of phosphorothioate and phosphodiester internucleotide linkages.

Immune antagonist oligonucleotides include, but are not limited to, oligonucleotides that block immunostimulation through endosomal toll-like receptors, RIG like receptors, STING, cGAS and inflammasomes. Toll-like receptors (TLRs) are pattern recognition receptors (PRRs) which play a crucial in the initiation of innate immune response by detecting potential harmful pathogens. Each TLR has a broad range of specificities, for example, TLR1, 2, 4 and 6 recognize bacterial lipids, TLR3, 7 and 8 recognize viral RNA, TLR9 recognizes bacterial DNA comprising a CG motif, and TLR5 and 10 recognize bacterial or parasite proteins. The design of such immune antagonist oligonucleotides is within the skill of ordinary artisans.

siRNA

In embodiments, the oligonucleotide of the functional domain is an siRNA. siRNAs comprise short double-stranded RNA from about 15 to about 50 nucleotides in length, preferably about 18 to about 36 nucleotides in length, that are targeted to the RNA.

In embodiments, the invention provides a CPN comprising a functional domain, a circularizing domain, wherein the functional domain comprises a siRNA; wherein the circularizing domain comprises a first nucleic acid molecule and a second nucleic acid molecule, wherein the first nucleic acid molecule and a second nucleic acid molecule are linked, directly or through a linker segment, to the siRNA of the functional domain; wherein the first nucleic acid molecule and the second nucleic acid molecule are independently 6 to 30 nucleotides in length, wherein the nucleotides of the first nucleic acid molecule and the second nucleic acid molecule are independently selected from RNA or DNA or a combination of RNA and DNA, and wherein the first nucleic acid molecule and the second nucleic acid molecule are complementary to and of opposite polarity to each other and hybridize to form a double-stranded section.

The terms “siRNA” and “short interfering RNA” are interchangeable and refer to single-stranded or double-stranded RNA molecules that are capable of inducing RNA interference. siRNA molecules typically have a duplex region that is between 18 and 36 base pairs in length. The design of such siRNAs is within the skill of ordinary artisans.

In one embodiment, when the oligonucleotide of the functional domain is an siRNA, the first nucleic acid molecule of the circularizing domain is attached at the 5′-end of the sense strand of the siRNA and the second nucleic acid molecule of the circularizing domain is attached at the 3′-end of the sense strand of the siRNA.

In one embodiment, when the oligonucleotide of the functional domain is an siRNA, the first nucleic acid molecule of the circularizing domain is attached at the 5′-end of the antisense strand of the siRNA and the second nucleic acid molecule of the circularizing domain is attached at the 3′-end of the antisense strand of the siRNA.

In one embodiment, when the oligonucleotide of the functional domain is an siRNA, the first nucleic acid molecule of the circularizing domain is attached at the 5′-end of the antisense strand of the siRNA and the second nucleic acid molecule of the circularizing domain is attached at the 5′-end of the sense strand of the siRNA.

In one embodiment, when the oligonucleotide of the functional domain is an siRNA, the first nucleic acid molecule of the circularizing domain is attached at the 3′-end of the antisense strand of the siRNA and the second nucleic acid molecule of the circularizing domain is attached at the 3′-end of the sense strand of the siRNA.

One or both strands of the siRNA of the invention can also comprise a 3′-overhang. A “3′ overhang” refers to at least one unpaired nucleotide extending from the 3′-end of an RNA strand. Thus, in one embodiment, the siRNA of the invention comprises at least one 3′ overhang of from one to about six nucleotides (which includes ribonucleotides or deoxynucleotides) in length, preferably from one to about five nucleotides in length, more preferably from one to about four nucleotides in length, and particularly preferably from about one to about two nucleotides in length.

In the case both strands of the siRNA molecule comprise a 3′ overhang, the length of the overhangs can be the same or different for each strand. In a most preferred embodiment, the 3′ overhang is present on both strands of the siRNA and is two nucleotides in length. In order to enhance the stability of the present siRNAs, the 3′ overhangs can also be stabilized against degradation. In one embodiment, the overhangs are stabilized by including purine nucleotides, such as adenosine or guanosine nucleotides.

Alternatively, substitution of pyrimidine nucleotides by modified analogues, e.g., substitution of uridine nucleotides in the 3′ overhangs with 2′-deoxythymidine, is tolerated and does not affect the efficiency of RNAi degradation. In particular, the absence of a 2′ hydroxyl in the 2′-deoxythymidine significantly enhances the nuclease resistance of the 3′ overhang in tissue culture medium.

The siRNAs of the invention can be targeted to any stretch of approximately 18-30, preferably 19-25 contiguous nucleotides of a target mRNA sequence. Techniques for selecting target sequences for siRNA are well known in the art. Thus, the sense strand of the present siRNA comprises a nucleotide sequence identical to any contiguous stretch of about 18 to about 30 nucleotides in the target mRNA.

### Pharmaceutical Composition

In certain embodiments, described herein are pharmaceutical compositions comprising one or more CPN compounds of the invention. In certain embodiments, the pharmaceutical composition comprises a pharmaceutically acceptable diluent or carrier. In certain embodiments, a pharmaceutical composition comprises a sterile saline solution and one or more CPN compounds. In certain embodiments, a pharmaceutical composition consists of a sterile saline solution and one or more CPN compounds. In certain embodiments, the sterile saline is pharmaceutical grade saline. In certain embodiments, a pharmaceutical composition comprises one or more CPN compounds and sterile water. In certain embodiments, a pharmaceutical composition consists of one CPN compound and sterile water. In certain embodiments, the sterile water is pharmaceutical grade water. In certain embodiments, a pharmaceutical composition comprises one or more CPN compounds and phosphate-buffered saline (PBS). In certain embodiments, a pharmaceutical composition consists of one or more CPN compounds and sterile PBS. In certain embodiments, the sterile PBS is pharmaceutical grade PBS.

In certain embodiments, pharmaceutical compositions comprise one or more CPN compounds and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, CPN compounds of the invention may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

### Conjugate Groups

In certain embodiments, the CPNs according to the invention optionally further comprise one or more conjugate groups. Conjugate groups consist of one or more conjugate moiety and a conjugate linker which links the one or more conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups are attached at the 3′-end of oligonucleotides. In certain embodiments, conjugate groups are attached near the 3′-end of oligonucleotides. In certain embodiments, conjugate groups are attached at the 5′-end of oligonucleotides. In certain embodiments, conjugate groups are attached near the 5′-end of oligonucleotides.

In any embodiment herein, the conjugate group comprises a GalNAc cluster comprising 1-3 GalNAc ligands.

In any embodiment herein, the conjugate linker consists of a single bond.

In any embodiment herein, the conjugate linker is cleavable.

In any embodiment herein, the conjugate linker comprises 1-3 linker-nucleosides.

In any embodiment herein, the conjugate group is attached to the CPN at the terminal end of the first nucleic acid molecule and/or the second nucleic acid molecule. The terminal end of the first or second nucleic acid molecule is the end not bound to the oligonucleotide of the CPN of the invention.

In certain embodiments, the CPNs are covalently attached to one or more conjugate groups. In certain embodiments, conjugate groups modify one or more properties of the CPNO, including but not limited to pharmacodynamics, pharmacokinetics, stability, binding, absorption, tissue distribution, cellular distribution, cellular uptake, charge and clearance. In certain embodiments, conjugate groups impart a new property on the CPN, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N. Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycerol-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety, a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

Conjugate moieties include, without limitation, intercalators, reporter molecules, polyamines, polyamides, peptides, carbohydrates, vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

Conjugate moieties are attached to the CPN through conjugate linkers. In certain embodiments, the conjugate linker is a single chemical bond (i.e., the conjugate moiety is attached directly to the CPN through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on the CPN and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl)cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

As used herein, linker-nucleosides are not considered to be part of the CPN in general or part of the circularizing domain or the functional domain in particular. Accordingly, the nucleotides of a linker-nucleosides are not counted toward the length of the CPN or the domains thereof and are not used in determining the percent complementarity of the oligonucleotide for the reference nucleic acid. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the CPN. For example, in certain circumstances CPNs comprising a particular conjugate moiety are better taken up by a particular cell type, but once the CPN has been taken up, it is desirable that the conjugate group be cleaved to release the unconjugated or parent CPN. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

### Use

Circular prodrug oligonucleotides of the invention could be useful for various mechanisms of action. These include antisense for mRNA, ncRNA, microRNA, lncRNA and splicing. Also, the double stranded circular prodrug structure could be used to deliver siRNA constructs. In addition, the circular prodrug structure could provide novel approach to deliver antisense for ADAR and CRISPR-based mechanisms. Furthermore, the circular prodrug structure could provide a novel approach to deliver antisense to disrupt structures of RNA to increase translation. Circular prodrug structures also provide defined structures to nucleic acids thereby allowing varying degree of interaction with PRRs and induced immune cascade.

For example, when the functional domain is an antisense oligonucleotide, CPNs according to the invention are also useful in therapeutic approaches in which inhibition of gene expression is desired. This can include, for example, inhibition of an endogenous gene (e.g., an oncogene) or an exogenous gene (e.g., a gene essential for growth and/or metabolism of a pathogen).

In embodiments, the invention provides a method for inhibiting gene expression comprising administering a circular prodrug nucleic acid as described herein or a composition comprising the circular prodrug nucleic acid.

In embodiments, the invention provides a method for inhibiting allele-specific gene expression comprising administering a circular prodrug nucleic acid as described herein or a composition comprising the circular prodrug nucleic acid.

The method according to the invention are useful for treating a subject having disease or disorder wherein inhibiting expression of a gene would be beneficial. In embodiments, the disease or disorder results from abnormal expression or product of a cellular gene.

In embodiments, the circular prodrug nucleic acid as described herein or a composition comprising the circular prodrug nucleic acid is administered locally.

In embodiments, the circular prodrug nucleic acid as described herein or a composition comprising the circular prodrug nucleic acid is administered systemically.

In embodiments, a method for modulating RNA processing comprising administering a CPN compound as described herein wherein the functional domain comprises an antisense oligonucleotide comprising 14 to 30 linked nucleotides having at least 12 contiguous nucleobases complementary to an equal length portion of a target RNA, wherein the antisense oligonucleotide comprises 1 to 3 DNA regions each region independently comprising from 2 to 5 consecutive deoxyribonucleotides and the remaining nucleotides are 2′-substituted, non-ionic or constrained sugar nucleotides, or combinations thereof. In embodiments, processing of RNA comprises splicing.

In embodiments, the invention provides a method for selecting a first mRNA transcript in a gene comprising at least two mRNA transcripts, the method comprising administering a circular prodrug nucleic acid as described herein or a composition comprising the circular prodrug nucleic acid.

In embodiments, the invention provides a method of treating a disease or disorder in a subject wherein modulating RNA processing would be beneficial to treat the subject, the method comprising administering a circular prodrug nucleic acid as described herein or a composition comprising the circular prodrug nucleic acid.

In embodiments, the invention provides a method of inducing nonsense mediated decay of a target RNA comprising administering a circular prodrug nucleic acid as described herein or a composition comprising the circular prodrug nucleic acid.

In embodiments, the invention provides a method of increasing a level of mRNA encoding a protein or a functional mRNA and increasing expression of the protein or the functional mRNA comprising administering a circular prodrug nucleic acid as described herein or a composition comprising the circular prodrug nucleic acid.

The circular prodrug nucleic acid of the invention may be administered alone or in combination with any other agent or therapy. Agents or therapies can be co-administered or administered concomitantly. Such agent or therapy may be useful for treating or preventing the disease or condition and does not diminish the gene expression modulation effect of the circular prodrug nucleic acid according to the invention. Agent(s) useful for treating or preventing the disease or condition includes, but is not limited to, small molecules, peptides vaccines, antigens, antibodies, preferably monoclonal antibodies, cytotoxic agents, kinase inhibitors, allergens, antibiotics, siRNA molecules, antisense oligonucleotides, TLR antagonist (e.g. antagonists of TLR3 and/or TLR7 and/or antagonists of TLR8 and/or antagonists of TLR9), chemotherapeutic agents (both traditional chemotherapy and modem targeted therapies), targeted therapeutic agents, activated cells, peptides, proteins, gene therapy vectors, peptide vaccines, protein vaccines, DNA vaccines, adjuvants, and co-stimulatory molecules (e.g. cytokines, chemokines, protein ligands, trans-activating factors, peptides or peptides comprising modified amino acids), or combinations thereof. Alternatively, the circular prodrug nucleic acid according to the invention can be administered in combination with other compounds (for example lipids or liposomes) to enhance the specificity or magnitude of the gene expression modulation of the circular prodrug nucleic acid according to the invention.

The circular prodrug nucleic acid of the invention may be administered by any suitable route, including, without limitation, parenteral, mucosal delivery, oral, sublingual, transdermal, topical, inhalation, intratumoral, intravenous, subcutaneous, intrathecal, intranasal, aerosol, intraocular, intratracheal, intrarectal, vaginal, by gene gun, dermal patch or in eye drop or mouthwash form. In any of the methods according to the invention, administration of the circular prodrug nucleic acid according to the invention, alone or in combination with any other agent, can be directly to a tissue or organ such as, but not limited to, the bladder, liver, lung or kidney. In certain embodiments, administration of the circular prodrug nucleic acid according to the invention, alone or in combination with any other agent, is by intramuscular administration. In certain embodiments, administration of circular prodrug nucleic acid according to the invention, alone or in combination with any other agent, is by mucosal administration. In certain embodiments, administration of circular prodrug nucleic acid according to the invention, alone or in combination with any other agent, is by oral administration. In certain embodiments, administration of circular prodrug nucleic acid according to the invention, alone or in combination with any other agent, is by intrarectal administration. In certain embodiments, administration of circular prodrug nucleic acid according to the invention, alone or in combination with any other agent, is by intrathecal administration. In certain embodiments, administration of circular prodrug nucleic acid according to the invention, alone or in combination with any other agent, is by intratumoral administration. In certain embodiments, administration of circular prodrug nucleic acid according to the invention, alone or in combination with any other agent, is by parenteral administration. In certain embodiments, administration of circular prodrug nucleic acid according to the invention, alone or in combination with any other agent, is by subcutaneous administration.

In embodiments, any of the circular prodrug nucleic acid described herein can be conjugated with a moiety that provides for site specific delivery of the CPN. In embodiments, such conjugates include, but are not limited to, an antibody, a peptide, a lipid, or a small molecule.

In embodiments, any of the circular prodrug nucleic acid described herein can be encapsulated with a moiety that provides for site specific delivery of the CPN. In embodiments, the CPN can be encapsulated in, for example, a lipid, lipid nanoparticles (LNP), or peptide macrocyclic structures.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. Administration of the circular prodrug nucleic acid according to the invention can be carried out using known procedures using an effective amount and for periods of time effective to reduce symptoms or surrogate markers of the disease. For example, an effective amount of a circular prodrug nucleic acid according to the invention for treating a disease and/or disorder could be that amount necessary to alleviate or reduce the symptoms, or delay or ameliorate a tumor, cancer, or bacterial, viral or fungal infection. In the context of administering a composition that modulates gene expression, an effective amount of a circular prodrug nucleic acid according to the invention is an amount sufficient to achieve the desired modulation as compared to the gene expression in the absence of the antisense oligonucleotide according to the invention. The effective amount for any particular application can vary depending on such factors as the disease or condition being treated, the particular oligonucleotide being administered, the size of the subject, or the severity of the disease or condition. One of ordinary skill in the art can empirically determine the effective amount of a particular circular prodrug nucleic acid without necessitating undue experimentation.

### Synthesis

CPNs described herein can be prepared by any suitable art recognized method including, but not limited to, H-phosphonate chemistry, phosphoramidite chemistry, or a combination of H-phosphonate chemistry and phosphoramidite chemistry (i.e., H-phosphonate chemistry for some cycles and phosphoramidite chemistry for other cycles), which can be carried out manually or by an automated synthesizer. The oligonucleotides of the invention may also be modified in a number of ways without compromising their ability to hybridize to their target (see e.g., Agrawal and Gait, Advances in Nucleic Acid Therapeutics, (2019)//doi.org/10.1039/9781788015714).

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout in the disclosure are incorporated by reference herein in their entirety.

Unless otherwise indicated, the following terms have the following meanings:

As used herein, “2′-deoxynucleoside” means a nucleoside comprising 2′-H(H) furanosyl sugar moiety, as found in naturally occurring deoxyribonucleic acids (DNA). In certain embodiments, a 2′-deoxynucleoside may comprise a modified nucleobase or may comprise an RNA nucleobase (uracil).

As used herein, “2′-substituted nucleoside” means a nucleoside comprising a 2′-substituted sugar moiety. As used herein, “2′-substituted” in reference to a sugar moiety means a sugar moiety comprising at least one 2′-substituent group other than H or OH.

As used herein, “5-methyl cytosine” means a cytosine modified with a methyl group attached to the 5-position. A 5-methyl cytosine is a modified nucleobase.

As used herein, the singular forms “a”, “an”, and “the” include both singular and plural referents unless the context clearly dictates otherwise.

As used herein, “administering” means providing a pharmaceutical agent to an animal.

As used herein, “animal” means a human or non-human animal.

As used herein, “individual in need thereof” refers to a human or non-human animal selected for treatment or therapy that is in need of such treatment or therapy.

As used herein, “antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound. In certain embodiments, antisense activity is an increase in the amount or expression of a target nucleic acid or protein encoded by such target nucleic acid compared to target nucleic acid levels or target protein levels in the absence of the antisense compound.

As used herein, “antisense compound” means an oligomeric compound capable of achieving at least one antisense activity.

As used herein, “ameliorate” in reference to a treatment means improvement in at least one symptom relative to the same symptom in the absence of the treatment. In certain embodiments, amelioration is the reduction in the severity or frequency of a symptom or the delayed onset or slowing of progression in the severity or frequency of a symptom. In certain embodiments, the symptom or hallmark is ataxia, neuropathy, and aggregate formation. In certain embodiments, amelioration of these symptoms results in improved motor function, reduced neuropathy, or reduction in number of aggregates.

As used herein, “bicyclic nucleoside” or “BNA” means a nucleoside comprising a bicyclic sugar moiety. As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the bicyclic sugar moiety does not comprise a furanosyl moiety.

As used herein, “chirally enriched population” means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom. Chirally enriched populations of molecules having multiple chiral centers within each molecule may contain one or more stereorandom chiral centers. In certain embodiments, the molecules are modified oligonucleotides. In certain embodiments, the molecules are compounds comprising modified oligonucleotides.

As used herein, “cleavable moiety” means a bond or group of atoms that is cleaved under physiological conditions, for example, inside a cell, an animal, or a human.

As used herein, the term “complementary” refers to a pair of nucleobases (or simply a “base”) that hydrogen bond to each other in preference to other heterocyclic bases under selected (e.g., physiological) conditions (or some degree of complementarity thereof as context may require in the instance of assessing “complementary-ness” of oligonucleotides). When the nucleobases are modified or unmodified, natural or synthetic purines and pyrimidines, the term “complementary” means complementary in the Watson Crick sense. In embodiments, at least 70% of the nucleobases of the oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases refers to nucleobases that are capable of forming hydrogen bonds with one another.

Complementary nucleobase pairs include adenine (A) and thymine (T), adenine (A) and uracil (U), cytosine (C) and guanine (G), 5-methyl cytosine (mC) and guanine (G). Complementary oligonucleotides and/or nucleic acids need not have nucleobase complementarity at each nucleoside. Rather, some mismatches are tolerated. As used herein, “fully complementary” or “100% complementary” in reference to oligonucleotides means that oligonucleotides are complementary to another oligonucleotide or nucleic acid at each nucleoside of the oligonucleotide.

As used herein, “conjugate group” means a group of atoms that is directly or indirectly attached to an oligonucleotide. Conjugate groups include a conjugate moiety and a conjugate linker that attaches the conjugate moiety to the oligonucleotide.

As used herein, “conjugate linker” means a group of atoms comprising at least one bond that connects a conjugate moiety to an oligonucleotide.

As used herein, “conjugate moiety” means a group of atoms that is attached to an oligonucleotide via a conjugate linker.

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

As used herein, “linker-nucleoside” means a nucleoside that links, either directly or indirectly, the CPN of the invention to a conjugate moiety. Linker-nucleosides are located within the conjugate linker and are not considered part of the CPN compound even if they are contiguous with the CPN.

As used herein, “gapmer” means a modified oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.” Unless otherwise indicated, “gapmer” refers to a sugar motif. Unless otherwise indicated, the sugar moieties of the nucleosides of the gap of a gapmer are unmodified 2′-deoxyfuranosyl. Thus, the term “MOE gapmer” indicates a gapmer having a sugar motif of 2′-MOE nucleosides in both wings and a gap of 2′-deoxynucleosides. Unless otherwise indicated, a MOE gapmer may comprise one or more modified internucleoside linkages and/or modified nucleobases and such modifications do not necessarily follow the gapmer pattern of the sugar modifications.

As used herein, “hotspot region” is a range of nucleobases on a target nucleic acid amenable to oligomeric compounds for reducing the amount or activity of the target nucleic acid as demonstrated in the examples herein below.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

As used herein, the term “internucleoside linkage” is the covalent linkage between adjacent nucleosides in an oligonucleotide. As used herein “modified internucleoside linkage” means any internucleoside linkage other than a phosphodiester internucleoside linkage. “Phosphorothioate linkage” is a modified internucleoside linkage in which one of the non-bridging oxygen atoms of a phosphodiester internucleoside linkage is replaced with a sulfur atom.

As used herein, the phrase “inhibiting the expression or activity” refers to a reduction or blockade of the expression or activity relative to the expression of activity in an untreated or control sample and does not necessarily indicate a total elimination of expression or activity.

As used herein, “non-bicyclic modified sugar moiety” means a modified sugar moiety that comprises a modification, such as a substituent, that does not form a bridge between two atoms of the sugar to form a second ring.

As used herein, “mismatch” or “non-complementary” means a nucleobase of a first oligonucleotide that is not complementary with the corresponding nucleobase of a second oligonucleotide or target nucleic acid when the first and second oligomeric compound are aligned.

As used herein, “MOE” means methoxyethyl. “2′-MOE” means a 2′-OCH₂CH₂OCH₃ group in place of the 2′ OH group of a ribosyl sugar moiety.

As used herein, “motif” means the pattern of unmodified and/or modified sugar moieties, nucleobases, and/or internucleoside linkages, in an oligonucleotide.

As used herein, “mRNA” means an RNA transcript that encodes a protein and includes pre-mRNA and mature mRNA unless otherwise specified.

As used herein, “nucleobase” means an unmodified nucleobase or a modified nucleobase. As used herein an “unmodified nucleobase” is adenine (A), thymine (T), cytosine (C), uracil (U), and guanine (G). As used herein, a “modified nucleobase” is a group of atoms other than unmodified A, T, C, U, or G capable of pairing with at least one unmodified nucleobase. A “5-methylcytosine” is an example of a modified nucleobase. A universal base is a modified nucleobase that can pair with any one of the five unmodified nucleobases. Modified bases, also referred to as heterocyclic base moieties, include other nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (including 5-bromo, 5-trifluoromethyl and other 5-substituted uracils and cytosines), 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

In certain embodiments, modified nucleobases are selected from: universal bases, hydrophobic bases, promiscuous bases, size-expanded bases, and fluorinated bases as defined herein. 5-substituted pyrimidines, 6-azapyrimidines and N-2, N-6 and O-6 substituted purines, including 2-aminopropyladenine, 5-propynyluracil; 5-propynylcytosine; 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, 3-deazaguanine and 3-deazaadenine. Further modified nucleobases include tricyclic pyrimidines such as phenoxazine cytidine ([5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. In certain embodiments, the modified nucleobase is a 5-methylcytosine.

Representative modified sugars include carbocyclic or acyclic sugars, sugars having substituent groups at one or more of their 2′, 3′ or 4′ positions and sugars having substituents in place of one or more hydrogen atoms of the sugar. In certain embodiments, the sugar is modified by having a substituent group at the 2′ position. In additional embodiments, the sugar is modified by having a substituent group at the 3′ position. In other embodiments, the sugar is modified by having a substituent group at the 4′ position. It is also contemplated that a sugar may have a modification at more than one of those positions, or that an antisense oligonucleotide may have one or more nucleotides with a sugar modification at one position and also one or more nucleotides with a sugar modification at a different position.

Sugar modifications contemplated in an oligonucleotide include, but are not limited to, a sugar substituent group selected from: OH; F; O-, S-, or N-alkyl; O-, S-, or N-alkenyl; O-, S or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. In some embodiments, these groups may be chosen from: O(CH₂)ₓOCH₃, O((CH₂)ₓO)_{y}CH₃, O(CH₂)ₓNH₂, O(CH₂)ₓCH₃, O(CH₂)ₓONH₂, and O(CH₂)ₓON((CH₂)ₓCH₃)₂, where x and y are independently from 1 to 10.

In some embodiments, the modified sugar comprises a substituent group selected from the following: C₁ to C₁₀ lower alkyl, substituted lower alkyl, alkenyl, alkynyl, alkaryl, aralkyl, O-alkaryl or O-aralkyl, SH, SCH₃, Cl, Br, CN, OCN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an antisense oligonucleotide, or a group for improving the pharmacodynamic properties of an antisense oligonucleotide, and other substituents having similar properties. In one embodiment, the modification includes 2′-methoxyethoxy (2′-O—CH₂CH₂OCH₃, which is also known as 2′-O-(2-methoxyethyl) or 2′-MOE) (Martin et al., 1995), that is, an alkoxyalkoxy group. Another modification includes 2′-dimethylaminooxyethoxy, that is, a O(CH₂)₂ON(CH₃)₂ group, also known as 2′-DMAOE and 2′-dimethylaminoethoxyethoxy (also known in the art as 2′-O-dimethyl-amino-ethoxy-ethyl or 2′-DMAEOE), that is, 2′-O—CH₂—O—CH₂—N(CH₃)₂.

Additional sugar substituent groups include allyl (—CH₂—CH═CH₂), —O-allyl CH₂—CH═CH₂), methoxy (—O—CH₃), aminopropoxy (—OCH₂CH₂CH₂NH₂), and fluoro (F). Sugar substituent groups on the 2′ position (2′-) may be in the arabino (up) position or ribo (down) position. One 2′-arabino modification is 2′-F. Other similar modifications may also be made at other positions on the oligomeric compound, particularly the 3′ position of the sugar on the 3′ terminal nucleoside or in 2′-5′ linked oligonucleotides and the 5′ position of 5′ terminal nucleotide. Oligomeric compounds may also have sugar mimetics, for example, cyclobutyl moieties, in place of the pentofuranosyl sugar. Examples of U.S. patents that disclose the preparation of modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, which are herein incorporated by reference in its entirety.

Representative sugar substituent groups include groups described in U.S. Patent Application Publication 2005/0261218, which is hereby incorporated by reference. In particular embodiments, the sugar modification is a 2′-O-Me modification, a 2′ F modification, a 2′ H modification, a 2′ amino modification, a 4′ thioribose modification or a phosphorothioate modification on the carboxy group linked to the carbon at position 6′, or combinations thereof.

In certain embodiments, a 2′-substituted non-bicyclic modified nucleoside comprises a sugar moiety comprising a non-bridging 2′-substituent group selected from: F, OCH₃, and OCH₂CH₂OCH₃.

Certain modified sugar moieties comprise a substituent that bridges two atoms of the furanosyl ring to form a second ring, resulting in a bicyclic sugar moiety. In certain such embodiments, the bicyclic sugar moiety comprises a bridge between the 4′ and the 2′ furanose ring atoms. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt”), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al, U.S. Pat. No. 7,569,686, Swayze et al., U.S. Pat. No. 7,741,457, and Swayze et al, U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al, U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al, J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)—C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—;

wherein:
x is 0, 1, or 2;
n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N3, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J Org. Chem., 1998, 63, 10035-10039; Srivastava et al., J Am. Chem. Soc, 2017, 129, 8362-8379; Wengel et al., U.S. Pat. No. 7,053,207; Imanishi et al., U.S. Pat. No. 6,268,490; Imanishi et al., U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

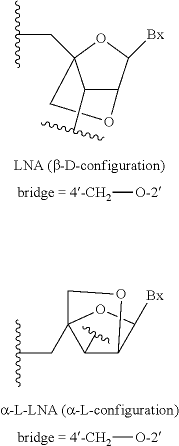

α-L-methyleneoxy (4′-CH2-0-2′) or α-L-LNA bicyclic nucleosides have been incorporated into oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, C J. Bioorg. & Med. Chem. 2002, 10, 841-854), fluoro HNA:

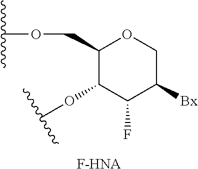

(“F-HNA”, see e.g., Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), and nucleosides comprising additional modified THP compounds having the formula:

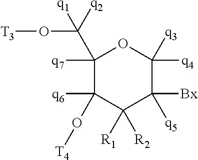

wherein, independently, for each of said modified THP nucleoside:
Bx is a nucleobase moiety;
T3 and T4 are each, independently, an internucleoside linking group linking the modified THP nucleoside to the remainder of an oligonucleotide or one of T3 and T4 is an internucleoside linking group linking the modified THP nucleoside to the remainder of an oligonucleotide and the other of T3 and T4 is H, a hydroxyl protecting group, a linked conjugate group, or a 5′ or 3′-terminal group;
q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

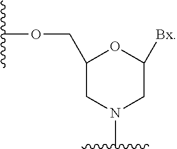

In certain embodiments, morpholinos may be modified, for example by adding or altering various substituent groups from the above morpholino structure. Such sugar surrogates are referred to herein as “modified morpholinos.” In embodiments, the morpholino or modified morpholino can further comprise a modified backbone, such as thiomorpholino or phosphorodiamidate morpholino (PMO), which are morpholino nucleoside(s) joined by thiophosphoramidate or phosphorodiamidate internucleotide linkages.

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides.

The nucleoside residues of the oligonucleotides of the functional or cyclizing domains can be coupled to each other by any of the numerous known internucleoside linkages. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS—P═S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Methods of preparation of phosphorous-containing and non-phosphorous-containing internucleoside linkages are well known to those skilled in the art.

Such internucleoside linkages include, without limitation, phosphodiester, phosphorothioate, phosphorodithioate, methylphosphonate, alkylphosphonate, alkylphosphonothioate, phosphotriester, phosphoramidate, siloxane, carbonate, carboalkoxy, acetamidate, carbamate, morpholino, borano, thioether, bridged phosphoramidate, bridged methylene phosphonate, bridged phosphorothioate, and sulfone internucleoside linkages. In some embodiments, the synthetic antisense oligonucleotides of the invention may comprise combinations of internucleotide linkages. In some embodiments, the synthetic antisense oligonucleotides of the invention may comprise combinations of phosphorothioate and phosphodiester internucleotide linkages. In some embodiments more than half but less that all of the internucleotide linkages are phosphorothioate internucleotide linkages. In some embodiments all of the internucleotide linkages are phosphorothioate internucleotide linkages.

Modified oligonucleotides comprising internucleoside linkages having a chiral center can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration.

In certain embodiments, the phosphorothioate linkages may be mixed Rp and Sp enantiomers, or they may be made stereoregular or substantially stereoregular in either Rp or Sp form. In embodiments where the linkages are mixed Rp and Sp enantiomers, the Rp and Sp forms may be at defined places within the oligonucleotide or randomly placed throughout the oligonucleotide.

As used herein, “nucleobase sequence” means the order of contiguous nucleobases in a nucleic acid or oligonucleotide independent of any sugar or internucleoside linkage modification.

As used herein, “nucleoside” means a compound comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. Modified nucleosides include abasic nucleosides, which lack a nucleobase. “Linked nucleosides” are nucleosides that are connected in a continuous sequence (i.e., no additional nucleosides are presented between those that are linked).

The term “nucleic acid,” in its broadest sense, includes any compound and/or substance that comprise a polymer of nucleotides. These polymers are often referred to as oligonucleotides or polynucleotides. Exemplary nucleic acids of the invention include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, a-LNA having an a-L-ribo configuration (a diastereomer of LNA), 2′-amino-LNA having a 2′-amino functionalization, and 2′-amino-a-LNA having a 2′-amino functionalization) or hybrids thereof.

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound.

As used herein, “oligonucleotide” means a strand of linked nucleosides of any length, including polynucleotides, connected via internucleoside linkages, wherein each nucleoside and internucleoside linkage may be modified or unmodified. Unless otherwise indicated, oligonucleotides consist of 8-50 linked nucleosides.

As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “pharmaceutically acceptable carrier or diluent” means any substance suitable for use in administering to an animal. Certain such carriers enable pharmaceutical compositions to be formulated as, for example, tablets, pills, capsules, liquids, gels, syrups, slurries, suspension and lozenges for the oral ingestion by a subject. In certain embodiments, a pharmaceutically acceptable carrier or diluent is sterile water; sterile saline; or sterile buffer solution.

As used herein “pharmaceutically acceptable salts” means physiologically and pharmaceutically acceptable salts of compounds, such as oligomeric compounds, i.e., salts that retain the desired biological activity of the parent compound and do not impart undesired toxicological effects thereto.

As used herein “pharmaceutical composition” means a mixture of substances suitable for administering to a subject. For example, a pharmaceutical composition may comprise an antisense compound and a sterile aqueous solution. In certain embodiments, a pharmaceutical composition shows activity in free uptake assay in certain cell lines.

As used herein, “phosphorus moiety” means a group of atoms comprising a phosphorus atom. In certain embodiments, a phosphorus moiety comprises a mono-, di-, or tri-phosphate, or phosphorothioate.

As used herein, “polypeptide” means a polymer of amino acid residues (natural or unnatural) linked together most often by peptide bonds. The term, as used herein, refers to proteins, polypeptides, and peptides of any size, structure, or function. In some instances the polypeptide encoded is smaller than about 50 amino acids and the polypeptide is then termed a peptide. If the polypeptide is a peptide, it will be at least about 2, 3, 4, or at least 5 amino acid residues long. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. They may also comprise single chain or multichain polypeptides such as antibodies or insulin and may be associated or linked. Most commonly disulfide linkages are found in multichain polypeptides. The term polypeptide may also apply to amino acid polymers in which one or more amino acid residues are an artificial chemical analogue of a corresponding naturally occurring amino acid.

The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. Ordinarily, variants will possess at least about 50% identity (homology) to a native or reference sequence, and preferably, they will be at least about 80%, more preferably at least about 90% identical (homologous) to a native or reference sequence.

As used herein “prodrug” means a therapeutic agent in a form outside the body that is converted to a different form within an animal or cells thereof. Typically, conversion of a prodrug within the animal is facilitated by the action of an enzyme (e.g., endogenous or viral enzyme) or chemicals present in cells or tissues and/or by physiologic conditions.

As used herein, “OMe” means methoxy. “2′-OMe” means a 2′-OCH₃ group in place of the 2′ OH group of a ribosyl sugar moiety.

The recitation of numerical ranges by endpoints includes all numbers and fractions subsumed within the respective ranges, as well as the recited endpoints.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, “reducing or inhibiting the amount or activity” refers to a reduction or blockade of the transcriptional expression or activity relative to the transcriptional expression or activity in an untreated or control sample and does not necessarily indicate a total elimination of transcriptional expression or activity.

As used herein, “self-complementary” in reference to an oligonucleotide means an oligonucleotide that at least partially hybridizes to itself.

As used herein, “standard cell assay” means the assay described in Example 1 and reasonable variations thereof.

As used herein, “stereorandom chiral center” in the context of a population of molecules of identical molecular formula means a chiral center having a random stereochemical configuration. For example, in a population of molecules comprising a stereorandom chiral center, the number of molecules having the (S) configuration of the stereorandom chiral center may be but is not necessarily the same as the number of molecules having the (R) configuration of the stereorandom chiral center. The stereochemical configuration of a chiral center is considered random when it is the result of a synthetic method that is not designed to control the stereochemical configuration. In certain embodiments, a stereorandom chiral center is a stereorandom phosphorothioate internucleoside linkage.

As used herein, “sugar moiety” means an unmodified sugar moiety or a modified sugar moiety. As used herein, “unmodified sugar moiety” means a 2′-OH(H) furanosyl moiety, as found in RNA (an “unmodified RNA sugar moiety”), or a 2′-H(H) moiety, as found in DNA (an “unmodified DNA sugar moiety”). Unmodified sugar moieties have one hydrogen at each of the 3′, and 4′ positions, an oxygen at the 3′ position, and two hydrogens at the 5′ position. As used herein, “modified sugar moiety” or “modified sugar” means a modified furanosyl sugar moiety or a sugar surrogate. As used herein, modified furanosyl sugar moiety means a furanosyl sugar comprising a non-hydrogen substituent in place of at least one hydrogen of an unmodified sugar moiety. In certain embodiments, a modified furanosyl sugar moiety is a 2′-substituted sugar moiety. Such modified furanosyl sugar moieties include bicyclic sugars and non-bicyclic sugars.

As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids.

As used herein, “target nucleic acid” and “target RNA” mean a nucleic acid that an antisense compound is designed to affect.

As used herein, “target region” means a portion of a target nucleic acid to which an oligomeric compound is designed to hybridize.

As used herein, “terminal group” means a chemical group or group of atoms that is covalently linked to a terminus of an oligonucleotide.

As used herein, “therapeutically effective amount” means an amount of a pharmaceutical agent that provides a therapeutic benefit to an animal. For example, a therapeutically effective amount improves a symptom of a disease.

As used herein, “treat”, “treatment”, or “treating” refers to administering a compound described herein to effect an alteration or improvement of a disease, disorder, or condition.

“Portion” means a defined number of contiguous (i.e., linked) nucleobases of a nucleic acid. In certain embodiments, a portion is a defined number of contiguous nucleobases of a target nucleic acid. In certain embodiments, a portion is a defined number of contiguous nucleobases of an antisense compound.

The term “co-administration” or “co-administered” generally refers to the administration of at least two different substances. Co-administration refers to simultaneous administration, as well as temporally spaced order of up to several days apart, of at least two different substances in any order, either in a single dose or separate doses.

The term “in combination with” generally means administering an oligonucleotide-based compound according to the invention and another agent useful for treating a disease or condition that does not abolish the activity of the compound in the course of treating a patient. Such administration may be done in any order, including simultaneous administration, as well as temporally spaced order from a few seconds up to several days apart. Such combination treatment may also include more than a single administration of the compound according to the invention and/or independently the other agent. The administration of the compound according to the invention and the other agent may be by the same or different routes.

The term “individual” or “subject” or “patient” generally refers to a mammal, such as a human. The term “mammal” is expressly intended to include warm blooded, vertebrate animals, including, without limitation, humans, non-human primates, rats, mice, cats, dogs, horses, cattle, cows, pigs, sheep and rabbits. As used herein, “individual in need thereof refers to a human or non-human animal selected for treatment or therapy that is in need of such treatment or therapy. As used herein, “inhibiting the expression or activity” refers to a reduction or blockade of the expression or activity of a RNA or protein and does not necessarily indicate a total elimination of expression or activity.

### EXAMPLES

### Synthesis of CPN Comprising an Antisense Oligonucleotide Functional Domain

Circular prodrug nucleic acids according to the invention can be synthesized by procedures that are well known in the art, such as phosphoramidate or H-phosphonate chemistry which can be carried out manually or by an automated synthesizer. For example, the oligonucleotides of the invention may be synthesized by a linear synthesis approach.

Compounds employed herein have been synthesized using phosphoramidite chemistry. These protocols are described in detail, for example in pubs.rsc.org/en/content/chapter/bk9781788012096-00453/978-1-78801-209-6, which is incorporated herein by reference.

### Inhibition of Target RNA by CPN Comprising an Antisense Oligonucleotide Functional Domain

Circular prodrug nucleic acids can be designed targeting a PCSK9 nucleic acid and tested for their effects on PCSK9 mRNA in vitro.

Hepa 1-6 cells can be cultured in DMEM medium plus 10% FBS and 100 U/ml Pen/Strep (cells from ATCC). The cells can be seeded and allowed to incubate overnight until they are ˜70% confluent at the time of transfection-˜100,000 cells/ml/12 well plate. Cell media can be changed and 900 μl can be added to each well. The oligonucleotides can be mixed with Lipofectamine in Opti-MEM medium, added to lipid (1:1 ratio) and incubated for 15-20 minutes. 100 μl can be added to each well for an antisense concentration of 100 nM. After a treatment period of about 16 to 48 hours, cells can be harvested for RNA and/or protein analysis.

Culture supernatants can be assayed for AK release cytotoxicity assay. Taqman probes for mPCSK9 and PPIB or HPRT1 (housekeeping controls) can be used (probes provided by ThermoFisher).

Circular prodrug nucleic acids can be designed targeting a PNPLA3 nucleic acid and tested for their effects on PNPLA3 mRNA in vitro.

Human HepG2 cells can be cultured following ATCC recommended condition and media (Eagle's Minimum Essential Medium with 10% FBS). Cells can be plated in PDL coated 96 well plates at 50 K/well density and reverse transfected with 0.6 ul/well RNAiMax and oligonucleotide compounds of indicated concentration. 24 hrs later, cells can be harvested using Cells to CT lysis reagent (ThermoFisher 4391851C). RNA can be reverse transcribed to the cDNA templates using the RT reagent kit (ThermoFisher A39110). Quantitative PCR can be performed using qPCR Master Mix (ThermoFisher 4444964). PNPLA3 expression level can be quantified using the PNPLA3-FAM probe (ThermoFisher 4351368 Assay ID: Hs00228747) and normalized with the housekeeping gene POLR2A (ThermoFisher, 4448491, Assay ID: Hs01108291). Data can be analyzed in GraphPad Prism.

To see if circularization of linear ASO with modified RNA in a splitmer format could provide further efficacy, the level of PNPLA3 knockdown using oligonucleotides having the same base sequence in the different splitmer format and chemistry arrangement can be compared:

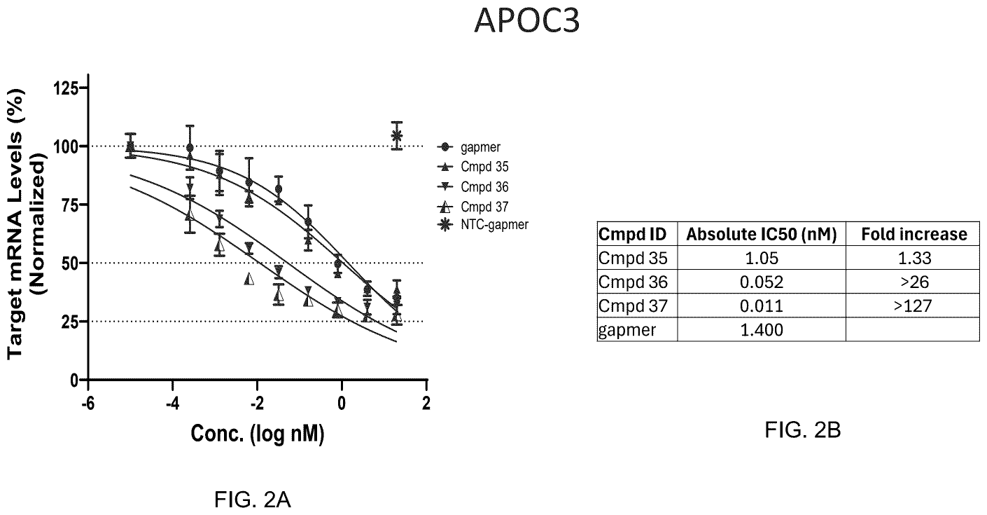

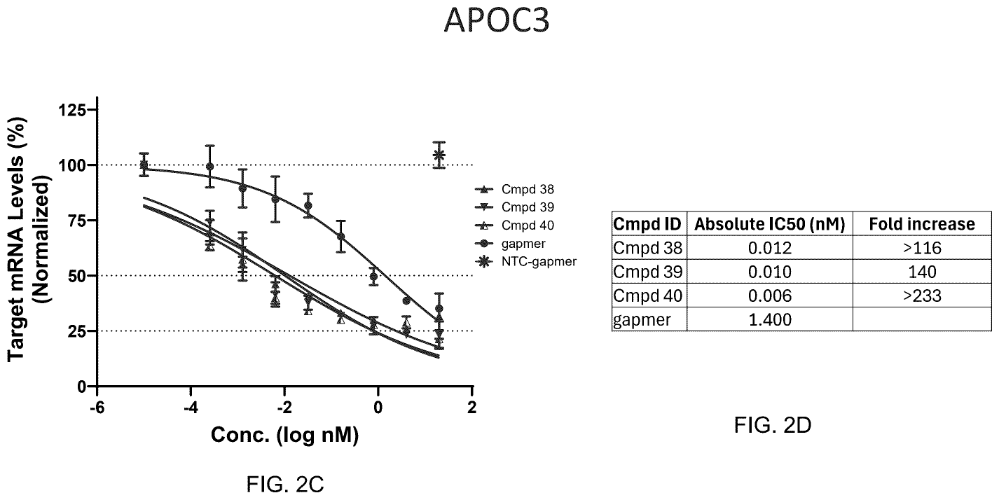

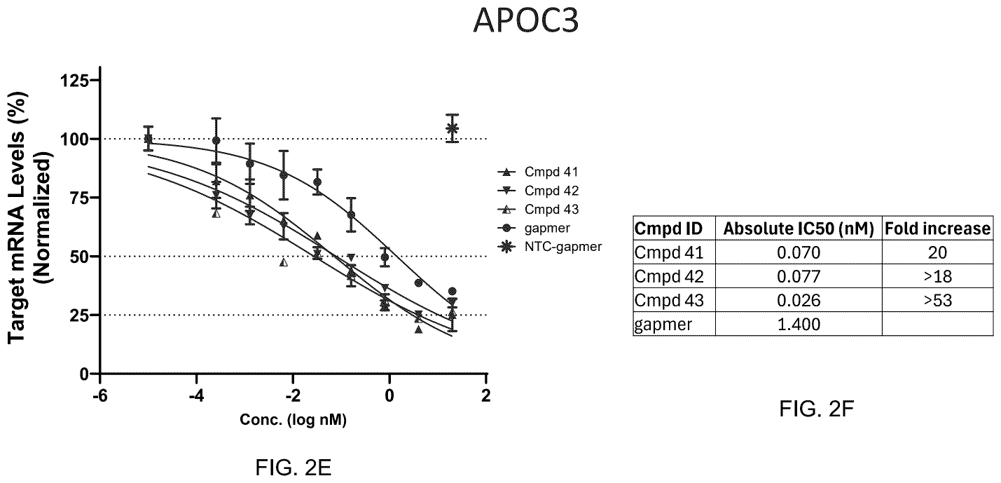

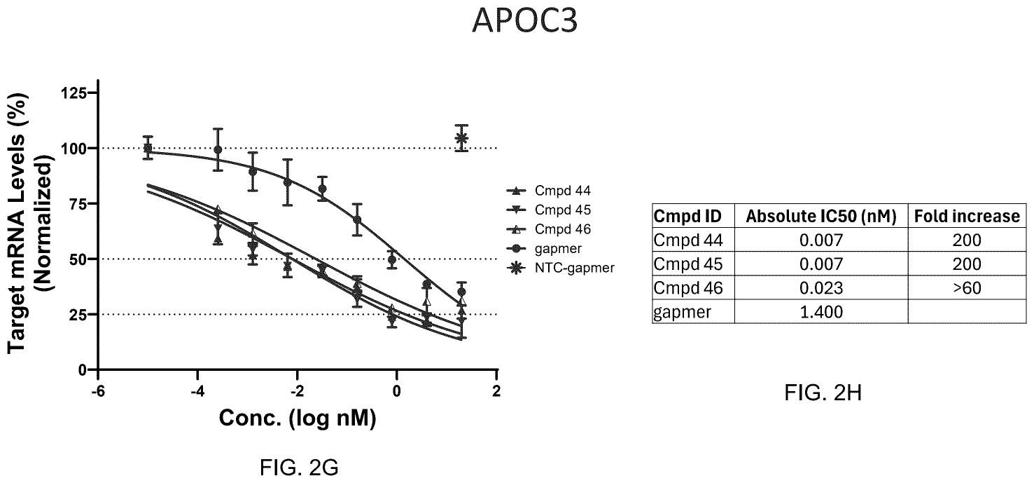

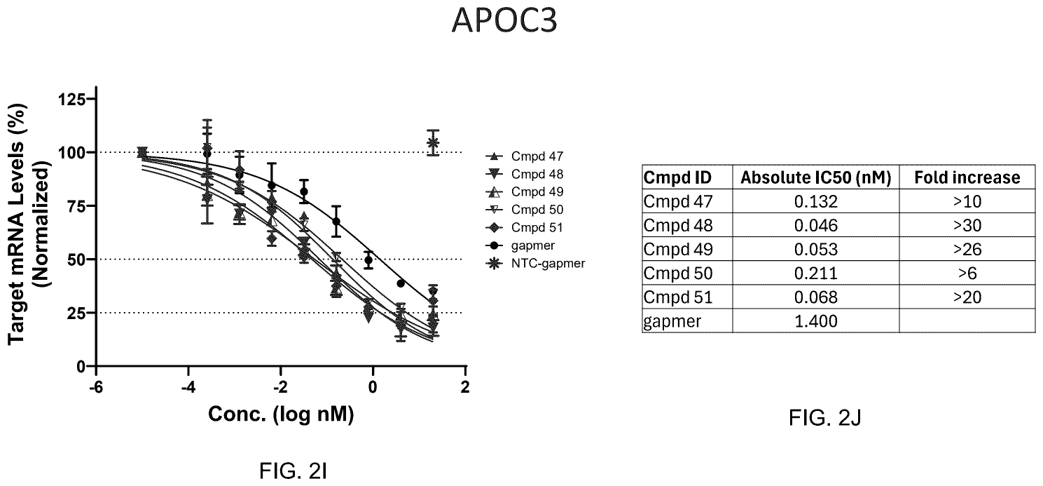

Circular prodrug nucleic acids were designed targeting a APOC3 nucleic acid and tested for their effects on APOC3 mRNA in vitro. Hep3B cells (human hepatic origin) were cultured following ATCC recommended conditions and media (Eagle's Minimum Essential Medium with 10% FBS). Cells were plated in 96 well plates at 20 K/well density and reverse transfected with 0.3 ul/well RNAiMax and antisense oligonucleotides (ASOs) at nine different concentrations (20, 4, 0.8, 0.16, 0.032, 0.0064, 0.00128, 0.000256, and 0.00001 nM). Control cells were transfected with 0.3 ul/well RNAiMax and media alone or 25 nM non-template control ASOs and incubated for 48 hours post-transfection. Gene expression was assayed using the Invitrogen™ TaqMan™ Fast Advanced Cells-to-CT™ Kit following the manufacturer's protocol (A35378, Invitrogen™). In brief, cells were harvested using the Cells to CT lysis reagent and immediately following total RNA was reverse transcribed into cDNA using the RT reagents. Quantitative polymerase chain reaction (qPCR) was performed via multiplexed reactions using qPCR Fast Advanced Master Mix and pre-designed primers and FAM-labelled probes (APOC3; Hs00163644_m1) and normalized using pre-designed primers and VIC-labeled probes (HPRT1; Hs02800695_m1) for the reference gene Hypoxanthine-guanine phosphoribosyltransferase (HPRT1). Data was plotted and analyzed using the ‘Absolute IC50’ nonlinear regression dose-response model of GraphPad Prism. Results are shown in FIG. 2A through FIG. 2J.

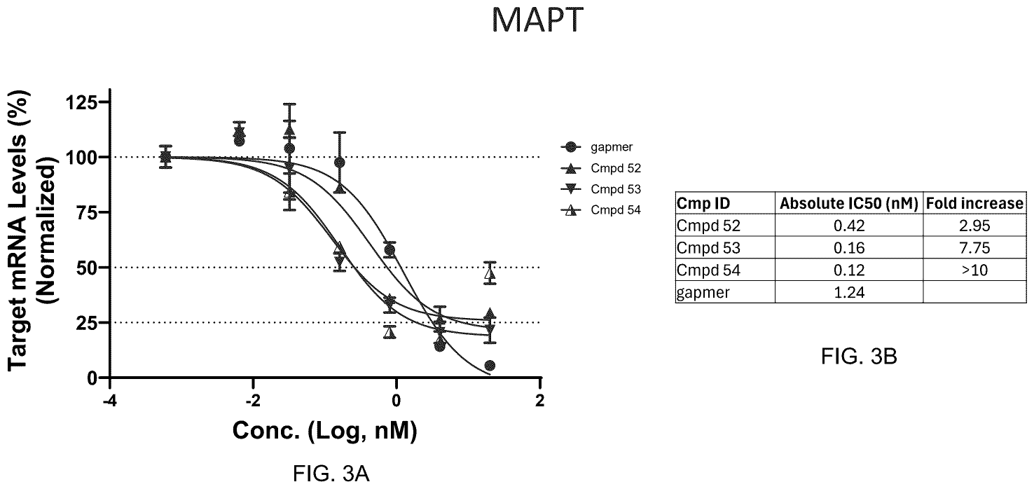

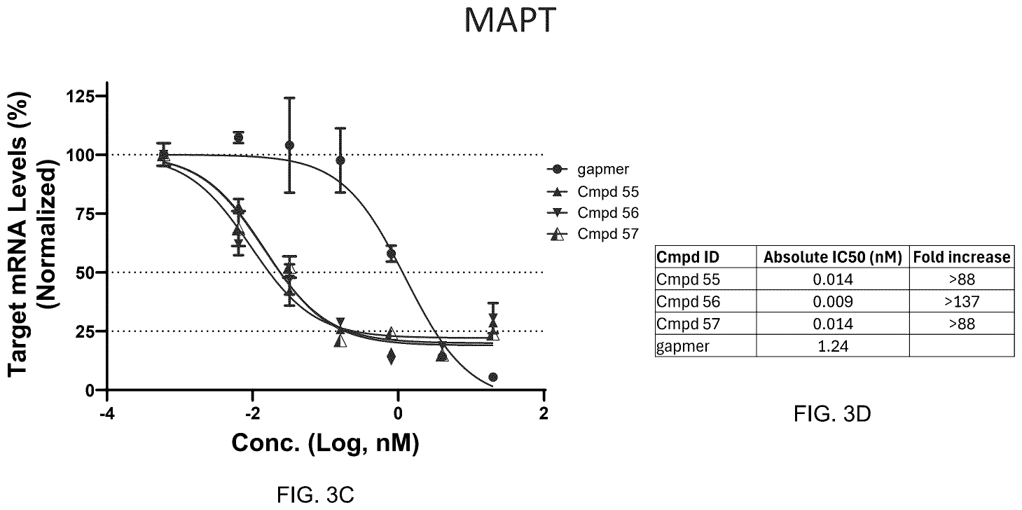

Circular prodrug nucleic acids were designed targeting a MAPT nucleic acid and tested for their effects on MAPT mRNA in vitro. U-251 MG cells were cultured following ATCC recommended conditions and media (Eagle's Minimum Essential Medium with 10% FBS). Cells were plated in 96 well plates at 20 K/well density and reverse transfected with 0.3 ul/well RNAiMax and cyclic structured antisense oligonucleotides (CSOs) at six different concentrations (25, 6.25, 1.56, 0.39, 0.10, and 0.024 nM). Control wells were transfected with 0.3 ul/well RNAiMax and media alone or 25 nM non-template control antisense oligonucleotides and incubated for 48 hours post-transfection. Gene expression was assayed using the Invitrogen™ TaqMan™ Fast Advanced Cells-to-CT™ Kit following the manufacturer's protocol (A35378, Invitrogen™). In brief, cells were harvested using the Cells to CT lysis reagent and immediately following total RNA was reverse transcribed into cDNA using the RT reagents. Quantitative polymerase chain reaction (qPCR) was performed via multiplexed reactions using qPCR Fast Advanced Master Mix and pre-designed primers and FAM-labelled probes (4351370, Invitrogen™) for the corresponding gene of interest (GOI) and normalized using pre-designed primers and VIC-labeled probes (#4448486, Invitrogen™) for the reference gene Hypoxanthine-guanine phosphoribosyltransferase (HPRT1). Data was plotted and analyzed using the ‘Absolute IC50’ nonlinear regression dose-response model of GraphPad Prism. Results are shown in FIG. 3A through FIG. 3D.

### Generation of Immune Response by CPN Comprising an Immunostimulatory Oligonucleotide Functional Domain

### Mouse Splenocyte Restimulation Assay

Spleens from C57BL/6J mice can be mechanically dispersed into single-cell suspensions, with ammonium chloride lysis buffer (Cat #420302, BioLegend, San Diego, CA) used to remove erythrocytes. Cell viability can be determined using vital dye stain and an automated cell counting system (Countess 3, Thermo Fisher, Waltham, MA). Following counting, 1×10⁵ viable mouse splenocytes can be seeded into each well of a 96-well flat bottom sterile tissue culture treated plate (Cat #3596, Corning, Glendale, AZ) in RPMI 1640 (Cat #A1049101, Thermo Fisher) containing 10% Fetal Bovine Serum (Cat #F2442, MilliporeSigma, Burlington, MA).

Immunostimulatory oligonucleotides (ISO) agonists for toll like receptor 9 (TLR9) and CPNs comprising TLR9 immunostimulatory oligonucleotide (ISO) functional domains can be synthesized using standard methodologies and provided as lyophilized preparations, which can be reconstituted in annealing buffer (10 mM Tris, 50 mM NaCl, 1 mM EDTA, pH 7.5) using a heat block incubation step (95° C., 5 minutes). Following annealing, sample concentrations can be determined using the absorbance method for oligonucleotide concentration determination using a NanoDrop spectrophotometer (Thermo Fisher). Control TLR9 agonists can be. Doses of CPNs or control agonists can be added to indicated wells, and cells can be stimulated for 24 hours in a 37° C., 5% CO2 incubator. Following the incubation period, cell culture supernatants can be harvested and analyzed using a custom multiplex cytokine/chemokine assay kit (U-PLEX custom biomarker assay, Cat #K15069M-2, Meso Scale Diagnostics, Rockville, MD) with data collection performed on a MSD S600 bioanalyzer (Meso Scale) and raw data analysis performed through MSD Discovery Workbench (Meso Scale).

To evaluate the activity of CPNs comprising immune antagonist oligonucleotide functional domains, such CPNs can be incubated alone or with a TLR9 agonist. Cytokines can be assessed 24 hours later.

While this invention has been particularly shown and described with references to preferred embodiments thereof, it will be understood by those skilled in the art that various changes in form and details may be made therein without departing from the scope of the invention encompassed by the appended claims.

1. A circular prodrug nucleic acid (CPN) comprising a functional domain and a circularizing domain,
wherein the CPN comprises a structure according to Formula I or Formula II: 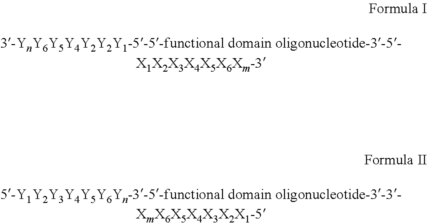
wherein
Y₁-Yₙ is a first nucleic acid molecule;
X₁-Xₘ is a second nucleic acid;
n is 0-44; and
m is 0-44;
wherein the nucleotides of the first nucleic acid molecule and the second nucleic acid molecule are independently selected from RNA or DNA, and wherein the first nucleic acid molecule and the second nucleic acid molecule are complementary to each other and of opposite polarity to each other and hybridize to form a double-stranded section.

2. The CPN according to claim 1, wherein the functional domain is selected from an antisense oligonucleotide, a microRNA (miRNA), a siRNA, a piRNA, a hnRNA, a ncRNA, a snRNA, a miRNA mimic, a sgRNA, an esiRNA, a shRNA, a lncRNA, a mRNA, an adeno-associated virus (AAV), a guideRNA of a CRISPR-based system, an ADAR-recruiting RNA of an adenosine deaminase acting on RNA (ADAR) system, a splicing oligonucleotide, an aptamer, an immunostimulatory oligonucleotide or an immune-inhibitory oligonucleotide.

3. The CPN according to claim 1, wherein the functional domain comprises an antisense oligonucleotide between 17 and 25 nucleotides in length and comprising at least 12 contiguous nucleobases complementary to an equal length portion of a target RNA sequence, wherein the antisense oligonucleotide compound comprises a 3′ domain and a 5′ domain, which is contiguous with the 3′ domain,
wherein the 3′ domain begins at the terminal nucleotide at the 3′ end and is 10 to 12 nucleotides in length and wherein each nucleotide is independently any deoxyribonucleotide;
wherein the 5′ domain begins at the first nucleotide following the 3′ domain and continues to the terminal nucleotide at the 5′ end, wherein the 5′ domain comprises unmodified deoxyribonucleotides, unmodified ribonucleotides, modified deoxyribonucleotides, modified ribonucleotides, or combinations thereof, provided that at least 3 nucleotides of the 5′ domain comprise a modified deoxyribonucleotide or modified ribonucleotide comprising a modified sugar and/or backbone.

4. The CPN according to claim 3, wherein the 3′ domain is 12 nucleotides in length and comprises nucleotides at positions 1, 2, 3, 4, 5, 6, 7, 8, 9, 10, 11, and 12 from the 3′ end.

5. The CPN according to claim 3, wherein the 3′ domain is 11 nucleotides in length and comprises nucleotides at positions 1, 2, 3, 4, 5, 6, 7, 8, 9, 10, and 11 from the 3′ end.

6. The CPN according to claim 3, wherein the 3′ domain is 10 nucleotides in length and comprises nucleotides at positions 1, 2, 3, 4, 5, 6, 7, 8, 9, and 10 from the 3′ end.

7. The CPN according to claim 1, wherein the first nucleic acid sequence is DNA and the second nucleic acid sequence is RNA.

8. The CPN according to claim 1, wherein the first nucleic acid sequence is RNA and the second nucleic acid sequence is DNA.

9. The CPN according to claim 7, wherein the nucleotides of the first nucleic acid sequence comprise unmodified deoxyribonucleotides and the nucleotides of the second nucleic acid sequence comprise unmodified ribonucleotides.

10. The CPN according to claim 8, wherein the nucleotides of the first nucleic acid sequence comprise unmodified ribonucleotides and the nucleotides of the second nucleic acid sequence comprise unmodified deoxyribonucleotides.

11. The CPN according to claim 3, wherein at least half of the nucleotides of the 5′ domain are a modified deoxyribonucleotide or modified ribonucleotide comprising a modified sugar and/or backbone.

12. The CPN according to claim 3, wherein all of the nucleotides of the 5′ domain are a modified ribonucleotide comprising a modified sugar and/or backbone.

13. The CPN according to claim 1, wherein the CPN comprises the structure of Formula I.

14. The CPN according to claim 1, wherein the CPN comprises the structure of Formula II.

15. The CPN according to claim 12, wherein the modified ribonucleotides comprise 2′-substituted ribonucleotides.

16. The CPN according to claim 15, wherein the 2′-substituted ribonucleotides are 2′-OMe ribonucleotides or 2′-MOE ribonucleotides.

17. The CPN according to claim 1, wherein the internucleotidic linkages of the oligonucleotides of the functional domain and/or the first nucleic acid and the second nucleic acid of the CPN comprise phosphorothioate internucleotide linkages, phosphodiester internucleotide linkages, or combinations thereof.

18. The CPN according to claim 1, wherein the internucleotidic linkages of the first nucleic acid and the second nucleic acid of the CPN comprise phosphorothioate internucleotide linkages.

19. The CPN according to claim 1, wherein the internucleotidic linkages of the first nucleic acid and the second nucleic acid of the CPN comprise phosphodiester internucleotide linkages.

20. The CPN according to claim 1, wherein the functional domain oligonucleotide is linked to the first nucleic acid molecule and the second nucleic acid molecule through a direct bond.

21. The CPN according to claim 1, wherein the first nucleic acid molecule and the second nucleic acid molecule of the circularizing domain hybridizes with each other to form a circular structure.

22. The CPN according to claim 1, wherein the first nucleic acid molecule and the second nucleic acid molecule are both RNA.

23. The CPN according to claim 21, wherein the double-stranded section formed by the first nucleic acid molecule and the second nucleic acid molecule is cleaved in situ resulting in linearizing the circular structure, thereby presenting the oligonucleotide of the functional domain and allowing it to perform its function.

24. A pharmaceutical composition comprising a CPN according to claim 1 and a pharmaceutically acceptable carrier.

25. The pharmaceutical composition according to claim 24, further comprising one or more agents selected from a small molecule, a peptide, a vaccine, an antigen, an antibody, a cytotoxic agent, a kinase inhibitor, an allergen, an antibiotic, an siRNA molecule, an antisense oligonucleotide, a TLR antagonist, a chemotherapeutic agent, a targeted therapeutic agent, an activated cell, a protein, a gene therapy vector, a peptide vaccine, a protein vaccine, a DNA vaccine, an adjuvant, and a co-stimulatory molecule, or combinations thereof.

26. A method for inhibiting gene expression comprising administering a CPN according to claim 1 or a composition thereof.

27. A method of treating a disease or disorder in a subject wherein modulating RNA would be beneficial to treat the subject, the method comprising administering a CPN according to claim 1 or a composition thereof.

28. A method of treating a disease or disorder in a subject wherein inhibiting gene expression would be beneficial to treat the subject, the method comprising administering a CPN according to claim 1 or a composition thereof.

29. A method of treating a disease or disorder in a subject wherein inducing an immune response would be beneficial to treat the subject, the method comprising administering a CPN according to claim 1 or a composition thereof.

30. A method of treating a disease or disorder in a subject wherein inhibiting an immune response would be beneficial to treat the subject, the method comprising administering a cyclic CPN according to claim 1 or a composition thereof.

31. A method of inducing nonsense mediated decay of a target RNA comprising administering a CPN according to claim 1 or a composition thereof.

32. A method of increasing a level of mRNA encoding a protein or a functional mRNA and increasing expression of the protein or the functional mRNA comprising administering a CPN according to claim 1 or a composition thereof.

33-36. (canceled)


SEQUENCE LISTING (192 sequences)

SEQ ID NO: 1 - DNA, 19 bp, synthetic construct
  source 1..19: mol_type = other DNA
ggtctcctccatcagcacc

SEQ ID NO: 2 - DNA, 20 bp, synthetic construct
  source 1..20: mol_type = other DNA
  modified_base 10: mod_base = OTHER; note = uracil
  modified_base 15..16: mod_base = OTHER; note = uracil
  misc_feature 15..16: note = RNA
  modified_base 1..16: mod_base = OTHER; note = 2'OME or 2'MOE ribonucleotide
  misc_feature 17..20: note = DNA phosphodiester
  misc_feature 1..9: note = DNA
  misc_feature 11..14: note = DNA
  misc_feature 17..20: note = DNA
  misc_feature 10: note = RNA
ggccaaacctcggcttacct

SEQ ID NO: 3 - DNA, 20 bp, synthetic construct
  source 1..20: mol_type = other DNA
tcttggttacatgaaatccc

SEQ ID NO: 4 - DNA, 20 bp, synthetic construct
  source 1..20: mol_type = other DNA
ggacattgccagtaatcgca

SEQ ID NO: 5 - DNA, 18 bp, synthetic construct
  source 1..18: mol_type = other DNA
  modified_base 1..18: mod_base = OTHER; note = 2'OME or 2'MOE ribonucleotide
tcactttcataatgctgg

SEQ ID NO: 6 - DNA, 20 bp, synthetic construct
  source 1..20: mol_type = other DNA
tgctccgttggtgcttgttc

SEQ ID NO: 7 - DNA, 20 bp, synthetic construct
  source 1..20: mol_type = other DNA
caggatacatttctacagct

SEQ ID NO: 8 - RNA, 12 nt, synthetic construct
  source 1..12: mol_type = other RNA
ctagtaccgtaa

SEQ ID NO: 9 - DNA, 12 bp, synthetic construct
  source 1..12: mol_type = other DNA
ctagtaccgtaa

SEQ ID NO: 10 - RNA, 12 nt, synthetic construct
  source 1..12: mol_type = other RNA
ttacggtactag

SEQ ID NO: 11 - DNA, 12 bp, synthetic construct
  source 1..12: mol_type = other DNA
ttacggtactag

SEQ ID NO: 12 - RNA, 17 nt, synthetic construct
  source 1..17: mol_type = other RNA
cgtatctagtaccgtaa

SEQ ID NO: 13 - DNA, 17 bp, synthetic construct
  source 1..17: mol_type = other DNA
cgtatctagtaccgtaa

SEQ ID NO: 14 - RNA, 16 nt, synthetic construct
  source 1..16: mol_type = other RNA
ttacgtactagatacg

SEQ ID NO: 15 - DNA, 16 bp, synthetic construct
  source 1..16: mol_type = other DNA
ttacgtactagatacg

SEQ ID NO: 16 - DNA, 14 bp, synthetic construct
  source 1..14: mol_type = other DNA
  misc_feature 1..14: note = DNA phosphodiester
tccctagtgtctaa

SEQ ID NO: 17 - DNA, 10 bp, synthetic construct
  source 1..10: mol_type = other DNA
  misc_feature 1..10: note = DNA phosphodiester
tccctagtgt

SEQ ID NO: 18 - skipped

SEQ ID NO: 19 - DNA, 14 bp, synthetic construct
  source 1..14: mol_type = other DNA
  misc_feature 1..14: note = DNA phosphodiester
ttagacactaggga

SEQ ID NO: 20 - DNA, 10 bp, synthetic construct
  source 1..10: mol_type = other DNA
  misc_feature 1..10: note = DNA phosphodiester
acactaggga

SEQ ID NO: 21 - skipped

SEQ ID NO: 22 - DNA, 14 bp, synthetic construct
  source 1..14: mol_type = other DNA
  misc_feature 1..14: note = DNA phosphodiester
agtcatctagacat

SEQ ID NO: 23 - DNA, 10 bp, synthetic construct
  source 1..10: mol_type = other DNA
  misc_feature 1..10: note = DNA phosphodiester
agtcatctag

SEQ ID NO: 24 - skipped

SEQ ID NO: 25 - DNA, 14 bp, synthetic construct
  source 1..14: mol_type = other DNA
  misc_feature 1..14: note = DNA phosphodiester
atgtctagatgact

SEQ ID NO: 26 - DNA, 10 bp, synthetic construct
  source 1..10: mol_type = other DNA
  misc_feature 1..10: note = DNA phosphodiester
atgtctagat

SEQ ID NO: 27 - skipped

SEQ ID NO: 28 - DNA, 14 bp, synthetic construct
  source 1..14: mol_type = other DNA
  misc_feature 1..4: note = DNA phosphodiester
  misc_feature 5..14: note = RNA phosphodiester
  misc_feature 1..4: note = DNA
  misc_feature 5..14: note = RNA
ttagacactaggga

SEQ ID NO: 29 - RNA, 10 nt, synthetic construct
  source 1..10: mol_type = other RNA
  misc_feature 1..10: note = RNA phosphodiester
acactaggga

SEQ ID NO: 30 - skipped

SEQ ID NO: 31 - DNA, 20 bp, synthetic construct
  source 1..20: mol_type = other DNA
  misc_feature 1..8: note = 2'MOE ribonucleotide with phosphorothioate linkage
caggatacatttctacagct

SEQ ID NO: 32 - DNA, 20 bp, synthetic construct
  source 1..20: mol_type = other DNA
gcttcttgtccagctttatt

SEQ ID NO: 33 - DNA, 20 bp, synthetic construct
  source 1..20: mol_type = other DNA
  misc_feature 1..5: note = 2'MOE ribonucleotide with phosphorothioate linkage
  misc_feature 16..20: note = 2'MOE ribonucleotide with phosphorothioate linkage
gcttcttgtccagctttatt

SEQ ID NO: 34 - DNA, 20 bp, synthetic construct
  source 1..20: mol_type = other DNA
  misc_feature 1..8: note = 2'MOE ribonucleotide with phosphorothioate linkage
gcttcttgtccagctttatt

SEQ ID NO: 35 - DNA, 18 bp, synthetic construct
  source 1..18: mol_type = other DNA
  misc_feature 1..7: note = 2'MOE ribonucleotide with phosphorothioate linkage
ccgttttcttaccaccct

SEQ ID NO: 36 - DNA, 20 bp, synthetic construct
  source 1..20: mol_type = other DNA
ccaaatcttataataactac

SEQ ID NO: 37 - DNA, 18 bp, synthetic construct
  source 1..18: mol_type = other DNA
ccgttttcttaccaccct

SEQ ID NO: 38 - DNA, 20 bp, synthetic construct
  source 1..20: mol_type = other DNA
gcctcagtctgcttcgcacc

SEQ ID NO: 39 - DNA, 20 bp, synthetic construct
  source 1..20: mol_type = other DNA
agcttcttgtccagctttat

SEQ ID NO: 40 - DNA, 20 bp, synthetic construct
  source 1..20: mol_type = other DNA
gctccttccactgatcctgc

SEQ ID NO: 41 - DNA, 20 bp, synthetic construct
  source 1..20: mol_type = other DNA
ccttggccacgccggcatcc

SEQ ID NO: 42 - DNA, 16 bp, synthetic construct
  source 1..16: mol_type = other DNA
ctatttggatgtcagc

SEQ ID NO: 43 - DNA, 20 bp, synthetic construct
  source 1..20: mol_type = other DNA
tgcaagtctcttggcaaaca

SEQ ID NO: 44 - DNA, 20 bp, synthetic construct
  source 1..20: mol_type = other DNA
tgccatttaatgagcttcac

SEQ ID NO: 45 - DNA, 18 bp, synthetic construct
  source 1..18: mol_type = other DNA
tcactttcataatgctgg

SEQ ID NO: 46 - DNA, 20 bp, synthetic construct
  source 1..20: mol_type = other DNA
  modified_base 4: mod_base = OTHER; note = uracil
  modified_base 16: mod_base = OTHER; note = uracil
  misc_feature 1..3: note = DNA
  misc_feature 5..15: note = DNA
  misc_feature 4: note = RNA
  misc_feature 16: note = RNA
  misc_feature 17..20: note = DNA
ccatactttccactgtaacg

SEQ ID NO: 47 - DNA, 20 bp, synthetic construct
  source 1..20: mol_type = other DNA
tgctccgttggtgcttgttc

SEQ ID NO: 48 - DNA, 20 bp, synthetic construct
  source 1..20: mol_type = other DNA
  misc_feature 1: note = DNA
  misc_feature 2: note = RNA
  misc_feature 3..20: note = DNA
  modified_base 2: mod_base = OTHER; note = uracil
ctcagtaacattgacaccac

SEQ ID NO: 49 - DNA, 20 bp, synthetic construct
  source 1..20: mol_type = other DNA
gcagaggtgaagcgaagtgc

SEQ ID NO: 50 - DNA, 16 bp, synthetic construct
  source 1..16: mol_type = other DNA
gggcctgtttagtcgc

SEQ ID NO: 51 - DNA, 20 bp, synthetic construct
  source 1..20: mol_type = other DNA
acggcattggtgcacagttt

SEQ ID NO: 52 - DNA, 20 bp, synthetic construct
  source 1..20: mol_type = other DNA
ctttattccaaagggcagct

SEQ ID NO: 53 - DNA, 18 bp, synthetic construct
  source 1..18: mol_type = other DNA
ccgttttcttaccaccct

SEQ ID NO: 54 - DNA, 19 bp, synthetic construct
  source 1..19: mol_type = other DNA
atggtttattccatggcca

SEQ ID NO: 55 - DNA, 21 bp, synthetic construct
  source 1..21: mol_type = other DNA
  misc_feature 1: note = DNA
  misc_feature 2..4: note = RNA
  misc_feature 5..21: note = DNA
  modified_base 2..4: mod_base = OTHER; note = uracil
ctttcagacacgggaacacgg

SEQ ID NO: 56 - DNA, 20 bp, synthetic construct
  source 1..20: mol_type = other DNA
cttcacatttcgatccaaca

SEQ ID NO: 57 - DNA, 18 bp, synthetic construct
  source 1..18: mol_type = other DNA
agttggagcaagattatc

SEQ ID NO: 58 - DNA, 22 bp, synthetic construct
  source 1..22: mol_type = other DNA
caatgccatcctggagttcctg

SEQ ID NO: 59 - DNA, 25 bp, synthetic construct
  source 1..25: mol_type = other DNA
gttgcctccggttctgaaggtgttc

SEQ ID NO: 60 - DNA, 30 bp, synthetic construct
  source 1..30: mol_type = other DNA
ctccaacatcaaggaagatggcatttctag

SEQ ID NO: 61 - DNA, 20 bp, synthetic construct
  source 1..20: mol_type = other DNA
  modified_base 1..8: mod_base = OTHER; note = 2'OME or 2'MOE ribonucleotide
gcctcagtctgcttcgcacc

SEQ ID NO: 62 - DNA, 20 bp, synthetic construct
  source 1..20: mol_type = other DNA
  modified_base 1..9: mod_base = OTHER; note = 2'OME or 2'MOE ribonucleotide
gcctcagtctgcttcgcacc

SEQ ID NO: 63 - DNA, 20 bp, synthetic construct
  source 1..20: mol_type = other DNA
  misc_feature 1..8: note = 2'MOE ribonucleotide with phosphorothioate linkage
gcctcagtctgcttcgcacc

SEQ ID NO: 64 - DNA, 20 bp, synthetic construct
  source 1..20: mol_type = other DNA
  misc_feature 1..9: note = 2'MOE ribonucleotide with phosphorothioate linkage
gcctcagtctgcttcgcacc

SEQ ID NO: 65 - DNA, 20 bp, synthetic construct
  source 1..20: mol_type = other DNA
  modified_base 1..8: mod_base = OTHER; note = 2'OME or 2'MOE ribonucleotide
tcttggttacatgaaatccc

SEQ ID NO: 66 - DNA, 20 bp, synthetic construct
  source 1..20: mol_type = other DNA
  modified_base 1..9: mod_base = OTHER; note = 2'OME or 2'MOE ribonucleotide
tcttggttacatgaaatccc

SEQ ID NO: 67 - DNA, 20 bp, synthetic construct
  source 1..20: mol_type = other DNA
  misc_feature 1..8: note = 2'MOE ribonucleotide with phosphorothioate linkage
tcttggttacatgaaatccc

SEQ ID NO: 68 - DNA, 20 bp, synthetic construct
  source 1..20: mol_type = other DNA
  misc_feature 1..9: note = 2'MOE ribonucleotide with phosphorothioate linkage
tcttggttacatgaaatccc

SEQ ID NO: 69 - DNA, 20 bp, synthetic construct
  source 1..20: mol_type = other DNA
  modified_base 1..8: mod_base = OTHER; note = 2'OME or 2'MOE ribonucleotide
agcttcttgtccagctttat

SEQ ID NO: 70 - DNA, 20 bp, synthetic construct
  source 1..20: mol_type = other DNA
  modified_base 1..9: mod_base = OTHER; note = 2'OME or 2'MOE ribonucleotide
agcttcttgtccagctttat

SEQ ID NO: 71 - DNA, 20 bp, synthetic construct
  source 1..20: mol_type = other DNA
  misc_feature 1..8: note = 2'MOE ribonucleotide with phosphorothioate linkage
agcttcttgtccagctttat

SEQ ID NO: 72 - DNA, 20 bp, synthetic construct
  source 1..20: mol_type = other DNA
  misc_feature 1..9: note = 2'MOE ribonucleotide with phosphorothioate linkage
agcttcttgtccagctttat

SEQ ID NO: 73 - DNA, 20 bp, synthetic construct
  source 1..20: mol_type = other DNA
  modified_base 1..8: mod_base = OTHER; note = 2'OME or 2'MOE ribonucleotide
gctccttccactgatcctgc

SEQ ID NO: 74 - DNA, 20 bp, synthetic construct
  source 1..20: mol_type = other DNA
  modified_base 1..9: mod_base = OTHER; note = 2'OME or 2'MOE ribonucleotide
gctccttccactgatcctgc

SEQ ID NO: 75 - DNA, 20 bp, synthetic construct
  source 1..20: mol_type = other DNA
  misc_feature 1..8: note = 2'MOE ribonucleotide with phosphorothioate linkage
gctccttccactgatcctgc

SEQ ID NO: 76 - DNA, 20 bp, synthetic construct
  source 1..20: mol_type = other DNA
  misc_feature 1..9: note = 2'MOE ribonucleotide with phosphorothioate linkage
gctccttccactgatcctgc

SEQ ID NO: 77 - DNA, 20 bp, synthetic construct
  source 1..20: mol_type = other DNA
  modified_base 1..8: mod_base = OTHER; note = 2'OME or 2'MOE ribonucleotide
ccttggccacgccggcatcc

SEQ ID NO: 78 - DNA, 20 bp, synthetic construct
  source 1..20: mol_type = other DNA
  modified_base 1..9: mod_base = OTHER; note = 2'OME or 2'MOE ribonucleotide
ccttggccacgccggcatcc

SEQ ID NO: 79 - DNA, 20 bp, synthetic construct
  source 1..20: mol_type = other DNA
  misc_feature 1..8: note = 2'MOE ribonucleotide with phosphorothioate linkage
ccttggccacgccggcatcc

SEQ ID NO: 80 - DNA, 20 bp, synthetic construct
  source 1..20: mol_type = other DNA
  misc_feature 1..9: note = 2'MOE ribonucleotide with phosphorothioate linkage
ccttggccacgccggcatcc

SEQ ID NO: 81 - DNA, 16 bp, synthetic construct
  source 1..16: mol_type = other DNA
  modified_base 1..4: mod_base = OTHER; note = 2'OME or 2'MOE ribonucleotide
ctatttggatgtcagc

SEQ ID NO: 82 - DNA, 16 bp, synthetic construct
  source 1..16: mol_type = other DNA
  modified_base 1..5: mod_base = OTHER; note = 2'OME or 2'MOE ribonucleotide
ctatttggatgtcagc

SEQ ID NO: 83 - DNA, 16 bp, synthetic construct
  source 1..16: mol_type = other DNA
  misc_feature 1..4: note = 2'MOE ribonucleotide with phosphorothioate linkage
ctatttggatgtcagc

SEQ ID NO: 84 - DNA, 16 bp, synthetic construct
  source 1..16: mol_type = other DNA
  misc_feature 1..5: note = 2'MOE ribonucleotide with phosphorothioate linkage
ctatttggatgtcagc

SEQ ID NO: 85 - DNA, 20 bp, synthetic construct
  source 1..20: mol_type = other DNA
  modified_base 1..8: mod_base = OTHER; note = 2'OME or 2'MOE ribonucleotide
ggacattgccagtaatcgca

SEQ ID NO: 86 - DNA, 20 bp, synthetic construct
  source 1..20: mol_type = other DNA
  modified_base 1..9: mod_base = OTHER; note = 2'OME or 2'MOE ribonucleotide
ggacattgccagtaatcgca

SEQ ID NO: 87 - DNA, 20 bp, synthetic construct
  source 1..20: mol_type = other DNA
  misc_feature 1..8: note = 2'MOE ribonucleotide with phosphorothioate linkage
ggacattgccagtaatcgca

SEQ ID NO: 88 - DNA, 20 bp, synthetic construct
  source 1..20: mol_type = other DNA
  misc_feature 1..9: note = 2'MOE ribonucleotide with phosphorothioate linkage
ggacattgccagtaatcgca

SEQ ID NO: 89 - DNA, 20 bp, synthetic construct
  source 1..20: mol_type = other DNA
  modified_base 1..8: mod_base = OTHER; note = 2'OME or 2'MOE ribonucleotide
tgcaagtctcttggcaaaca

SEQ ID NO: 90 - DNA, 20 bp, synthetic construct
  source 1..20: mol_type = other DNA
  modified_base 1..9: mod_base = OTHER; note = 2'OME or 2'MOE ribonucleotide
tgcaagtctcttggcaaaca

SEQ ID NO: 91 - DNA, 20 bp, synthetic construct
  source 1..20: mol_type = other DNA
  misc_feature 1..8: note = 2'MOE ribonucleotide with phosphorothioate linkage
tgcaagtctcttggcaaaca

SEQ ID NO: 92 - DNA, 20 bp, synthetic construct
  source 1..20: mol_type = other DNA
  misc_feature 1..9: note = 2'MOE ribonucleotide with phosphorothioate linkage
tgcaagtctcttggcaaaca

SEQ ID NO: 93 - DNA, 20 bp, synthetic construct
  source 1..20: mol_type = other DNA
  modified_base 1..8: mod_base = OTHER; note = 2'OME or 2'MOE ribonucleotide
tgccatttaatgagcttcac

SEQ ID NO: 94 - DNA, 20 bp, synthetic construct
  source 1..20: mol_type = other DNA
  modified_base 1..9: mod_base = OTHER; note = 2'OME or 2'MOE ribonucleotide
tgccatttaatgagcttcac

SEQ ID NO: 95 - DNA, 20 bp, synthetic construct
  source 1..20: mol_type = other DNA
  misc_feature 1..8: note = 2'MOE ribonucleotide with phosphorothioate linkage
tgccatttaatgagcttcac

SEQ ID NO: 96 - DNA, 20 bp, synthetic construct
  source 1..20: mol_type = other DNA
  misc_feature 1..9: note = 2'MOE ribonucleotide with phosphorothioate linkage
tgccatttaatgagcttcac

SEQ ID NO: 97 - DNA, 20 bp, synthetic construct
  source 1..20: mol_type = other DNA
  modified_base 1..8: mod_base = OTHER; note = 2'OME or 2'MOE ribonucleotide
caggatacatttctacagct

SEQ ID NO: 98 - DNA, 20 bp, synthetic construct
  source 1..20: mol_type = other DNA
  modified_base 1..7: mod_base = OTHER; note = 2'OME or 2'MOE ribonucleotide
caggatacatttctacagct

SEQ ID NO: 99 - DNA, 20 bp, synthetic construct
  source 1..20: mol_type = other DNA
  misc_feature 1..8: note = 2'MOE ribonucleotide with phosphorothioate linkage
caggatacatttctacagct

SEQ ID NO: 100 - DNA, 20 bp, synthetic construct
  source 1..20: mol_type = other DNA
  misc_feature 1..7: note = 2'MOE ribonucleotide with phosphorothioate linkage
caggatacatttctacagct

SEQ ID NO: 101 - DNA, 20 bp, synthetic construct
  source 1..20: mol_type = other DNA
  misc_feature 1: note = 2'MOE ribonucleotide with phosphorothioate linkage
  misc_feature 3: note = 2'MOE ribonucleotide with phosphorothioate linkage
  misc_feature 5: note = 2'MOE ribonucleotide with phosphorothioate linkage
  misc_feature 7..8: note = 2'MOE ribonucleotide with phosphorothioate linkage
caggatacatttctacagct

SEQ ID NO: 102 - DNA, 20 bp, synthetic construct
  source 1..20: mol_type = other DNA
  misc_feature 1: note = 2'MOE ribonucleotide with phosphorothioate linkage
  misc_feature 3: note = 2'MOE ribonucleotide with phosphorothioate linkage
  misc_feature 5: note = 2'MOE ribonucleotide with phosphorothioate linkage
  misc_feature 7: note = 2'MOE ribonucleotide with phosphorothioate linkage
caggatacatttctacagct

SEQ ID NO: 103 - DNA, 20 bp, synthetic construct
  source 1..20: mol_type = other DNA
  modified_base 1..8: mod_base = OTHER; note = 2'OME or 2'MOE ribonucleotide
caggatacatttctacagct

SEQ ID NO: 104 - DNA, 20 bp, synthetic construct
  source 1..20: mol_type = other DNA
  modified_base 1..7: mod_base = OTHER; note = 2'OME or 2'MOE ribonucleotide
caggatacatttctacagct

SEQ ID NO: 105 - DNA, 20 bp, synthetic construct
  source 1..20: mol_type = other DNA
  misc_feature 1..8: note = 2'MOE ribonucleotide with phosphorothioate linkage
caggatacatttctacagct

SEQ ID NO: 106 - DNA, 20 bp, synthetic construct
  source 1..20: mol_type = other DNA
  misc_feature 1..7: note = 2'MOE ribonucleotide with phosphorothioate linkage
caggatacatttctacagct

SEQ ID NO: 107 - DNA, 20 bp, synthetic construct
  source 1..20: mol_type = other DNA
  misc_feature 1: note = 2'MOE ribonucleotide with phosphorothioate linkage
  misc_feature 3: note = 2'MOE ribonucleotide with phosphorothioate linkage
  misc_feature 5: note = 2'MOE ribonucleotide with phosphorothioate linkage
  misc_feature 7..8: note = 2'MOE ribonucleotide with phosphorothioate linkage
caggatacatttctacagct

SEQ ID NO: 108 - DNA, 20 bp, synthetic construct
  source 1..20: mol_type = other DNA
  misc_feature 1: note = 2'MOE ribonucleotide with phosphorothioate linkage
  misc_feature 3: note = 2'MOE ribonucleotide with phosphorothioate linkage
  misc_feature 5: note = 2'MOE ribonucleotide with phosphorothioate linkage
  misc_feature 7: note = 2'MOE ribonucleotide with phosphorothioate linkage
caggatacatttctacagct

SEQ ID NO: 109 - DNA, 20 bp, synthetic construct
  source 1..20: mol_type = other DNA
  modified_base 1..8: mod_base = OTHER; note = 2'OME or 2'MOE ribonucleotide
  modified_base 4: mod_base = OTHER; note = uracil
  modified_base 16: mod_base = OTHER; note = uracil
  misc_feature 1..3: note = DNA
  misc_feature 4: note = RNA
  misc_feature 5..15: note = DNA
  misc_feature 16: note = RNA
  misc_feature 17..20: note = DNA
ccatactttccactgtaacg

SEQ ID NO: 110 - DNA, 20 bp, synthetic construct
  source 1..20: mol_type = other DNA
  misc_feature 1..8: note = 2'MOE ribonucleotide with phosphorothioate linkage
  modified_base 4: mod_base = OTHER; note = uracil
  modified_base 16: mod_base = OTHER; note = uracil
  misc_feature 1..3: note = DNA
  misc_feature 4: note = RNA
  misc_feature 5..15: note = DNA
  misc_feature 16: note = RNA
  misc_feature 17..20: note = DNA
ccatactttccactgtaacg

SEQ ID NO: 111 - DNA, 20 bp, synthetic construct
  source 1..20: mol_type = other DNA
  misc_feature 1..9: note = 2'MOE ribonucleotide with phosphorothioate linkage
  modified_base 4: mod_base = OTHER; note = uracil
  modified_base 16: mod_base = OTHER; note = uracil
  misc_feature 1..3: note = DNA
  misc_feature 4: note = RNA
  misc_feature 5..15: note = DNA
  misc_feature 16: note = RNA
  misc_feature 17..20: note = DNA
ccatactttccactgtaacg

SEQ ID NO: 112 - DNA, 20 bp, synthetic construct
  source 1..20: mol_type = other DNA
  modified_base 1..9: mod_base = OTHER; note = 2'OME or 2'MOE ribonucleotide
tgctccgttggtgcttgttc

SEQ ID NO: 113 - DNA, 20 bp, synthetic construct
  source 1..20: mol_type = other DNA
  modified_base 1..8: mod_base = OTHER; note = 2'OME or 2'MOE ribonucleotide
tgctccgttggtgcttgttc

SEQ ID NO: 114 - DNA, 20 bp, synthetic construct
  source 1..20: mol_type = other DNA
  misc_feature 1..9: note = 2'MOE ribonucleotide with phosphorothioate linkage
tgctccgttggtgcttgttc

SEQ ID NO: 115 - DNA, 20 bp, synthetic construct
  source 1..20: mol_type = other DNA
  misc_feature 1..8: note = 2'MOE ribonucleotide with phosphorothioate linkage
tgctccgttggtgcttgttc

SEQ ID NO: 116 - DNA, 20 bp, synthetic construct
  source 1..20: mol_type = other DNA
  modified_base 1..9: mod_base = OTHER; note = 2'OME or 2'MOE ribonucleotide
  modified_base 2: mod_base = OTHER; note = uracil
  misc_feature 1: note = DNA
  misc_feature 2: note = RNA
  misc_feature 3..20: note = DNA
ctcagtaacattgacaccac

SEQ ID NO: 117 - DNA, 20 bp, synthetic construct
  source 1..20: mol_type = other DNA
  modified_base 1..10: mod_base = OTHER; note = 2'OME or 2'MOE ribonucleotide
  misc_feature 1: note = DNA
  misc_feature 2: note = RNA
  misc_feature 3..20: note = DNA
  modified_base 2: mod_base = OTHER; note = uracil
ctcagtaacattgacaccac

SEQ ID NO: 118 - DNA, 20 bp, synthetic construct
  source 1..20: mol_type = other DNA
  misc_feature 1..10: note = 2'MOE ribonucleotide with phosphorothioate linkage
  misc_feature 1: note = DNA
  misc_feature 2: note = RNA
  misc_feature 3..20: note = DNA
  modified_base 2: mod_base = OTHER; note = uracil
ctcagtaacattgacaccac

SEQ ID NO: 119 - DNA, 20 bp, synthetic construct
  source 1..20: mol_type = other DNA
  misc_feature 1..9: note = 2'MOE ribonucleotide with phosphorothioate linkage
  misc_feature 1: note = DNA
  misc_feature 2: note = RNA
  misc_feature 3..20: note = DNA
  modified_base 2: mod_base = OTHER; note = uracil
ctcagtaacattgacaccac

SEQ ID NO: 120 - DNA, 20 bp, synthetic construct
  source 1..20: mol_type = other DNA
  modified_base 1..9: mod_base = OTHER; note = 2'OME or 2'MOE ribonucleotide
gcagaggtgaagcgaagtgc

SEQ ID NO: 121 - DNA, 20 bp, synthetic construct
  source 1..20: mol_type = other DNA
  modified_base 1..8: mod_base = OTHER; note = 2'OME or 2'MOE ribonucleotide
gcagaggtgaagcgaagtgc

SEQ ID NO: 122 - DNA, 20 bp, synthetic construct
  source 1..20: mol_type = other DNA
  misc_feature 1..9: note = 2'MOE ribonucleotide with phosphorothioate linkage
gcagaggtgaagcgaagtgc

SEQ ID NO: 123 - DNA, 20 bp, synthetic construct
  source 1..20: mol_type = other DNA
  misc_feature 1..8: note = 2'MOE ribonucleotide with phosphorothioate linkage
gcagaggtgaagcgaagtgc

SEQ ID NO: 124 - DNA, 16 bp, synthetic construct
  source 1..16: mol_type = other DNA
  modified_base 1..5: mod_base = OTHER; note = 2'OME or 2'MOE ribonucleotide
gggcctgtttagtcgc

SEQ ID NO: 125 - DNA, 16 bp, synthetic construct
  source 1..16: mol_type = other DNA
  modified_base 1..4: mod_base = OTHER; note = 2'OME or 2'MOE ribonucleotide
gggcctgtttagtcgc

SEQ ID NO: 126 - DNA, 16 bp, synthetic construct
  source 1..16: mol_type = other DNA
  misc_feature 1..5: note = 2'MOE ribonucleotide with phosphorothioate linkage
gggcctgtttagtcgc

SEQ ID NO: 127 - DNA, 16 bp, synthetic construct
  source 1..16: mol_type = other DNA
  misc_feature 1..4: note = 2'MOE ribonucleotide with phosphorothioate linkage
gggcctgtttagtcgc

SEQ ID NO: 128 - DNA, 20 bp, synthetic construct
  source 1..20: mol_type = other DNA
  modified_base 1..9: mod_base = OTHER; note = 2'OME or 2'MOE ribonucleotide
cacaaacaagctggtcggtt

SEQ ID NO: 129 - DNA, 20 bp, synthetic construct
  source 1..20: mol_type = other DNA
  modified_base 1..8: mod_base = OTHER; note = 2'OME or 2'MOE ribonucleotide
cacaaacaagctggtcggtt

SEQ ID NO: 130 - DNA, 20 bp, synthetic construct
  source 1..20: mol_type = other DNA
  misc_feature 1..9: note = 2'MOE ribonucleotide with phosphorothioate linkage
cacaaacaagctggtcggtt

SEQ ID NO: 131 - DNA, 20 bp, synthetic construct
  source 1..20: mol_type = other DNA
  misc_feature 1..8: note = 2'MOE ribonucleotide with phosphorothioate linkage
cacaaacaagctggtcggtt

SEQ ID NO: 132 - DNA, 20 bp, synthetic construct
  source 1..20: mol_type = other DNA
  modified_base 1..8: mod_base = OTHER; note = 2'OME or 2'MOE ribonucleotide
acggcattggtgcacagttt

SEQ ID NO: 133 - DNA, 20 bp, synthetic construct
  source 1..20: mol_type = other DNA
  modified_base 1..9: mod_base = OTHER; note = 2'OME or 2'MOE ribonucleotide
acggcattggtgcacagttt

SEQ ID NO: 134 - DNA, 20 bp, synthetic construct
  source 1..20: mol_type = other DNA
  misc_feature 1..8: note = 2'MOE ribonucleotide with phosphorothioate linkage
acggcattggtgcacagttt

SEQ ID NO: 135 - DNA, 20 bp, synthetic construct
  source 1..20: mol_type = other DNA
  misc_feature 1..9: note = 2'MOE ribonucleotide with phosphorothioate linkage
acggcattggtgcacagttt

SEQ ID NO: 136 - DNA, 18 bp, synthetic construct
  source 1..18: mol_type = other DNA
  modified_base 1..14: mod_base = OTHER; note = 2'OME or 2'MOE ribonucleotide
tcactttcataatgctgg

SEQ ID NO: 137 - DNA, 18 bp, synthetic construct
  source 1..18: mol_type = other DNA
  misc_feature 1..14: note = 2'MOE ribonucleotide with phosphorothioate linkage
tcactttcataatgctgg

SEQ ID NO: 138 - DNA, 20 bp, synthetic construct
  source 1..20: mol_type = other DNA
  modified_base 1..8: mod_base = OTHER; note = 2'OME or 2'MOE ribonucleotide
ctttattccaaagggcagct

SEQ ID NO: 139 - DNA, 20 bp, synthetic construct
  source 1..20: mol_type = other DNA
  modified_base 1..9: mod_base = OTHER; note = 2'OME or 2'MOE ribonucleotide
ctttattccaaagggcagct

SEQ ID NO: 140 - DNA, 20 bp, synthetic construct
  source 1..20: mol_type = other DNA
  misc_feature 1..8: note = 2'MOE ribonucleotide with phosphorothioate linkage
ctttattccaaagggcagct

SEQ ID NO: 141 - DNA, 20 bp, synthetic construct
  source 1..20: mol_type = other DNA
  misc_feature 1..9: note = 2'MOE ribonucleotide with phosphorothioate linkage
ctttattccaaagggcagct

SEQ ID NO: 142 - DNA, 18 bp, synthetic construct
  source 1..18: mol_type = other DNA
  modified_base 1..7: mod_base = OTHER; note = 2'OME or 2'MOE ribonucleotide
ccgttttcttaccaccct

SEQ ID NO: 143 - DNA, 18 bp, synthetic construct
  source 1..18: mol_type = other DNA
  modified_base 1..6: mod_base = OTHER; note = 2'OME or 2'MOE ribonucleotide
ccgttttcttaccaccct

SEQ ID NO: 144 - DNA, 18 bp, synthetic construct
  source 1..18: mol_type = other DNA
  misc_feature 1..7: note = 2'MOE ribonucleotide with phosphorothioate linkage
ccgttttcttaccaccct

SEQ ID NO: 145 - DNA, 18 bp, synthetic construct
  source 1..18: mol_type = other DNA
  misc_feature 1..6: note = 2'MOE ribonucleotide with phosphorothioate linkage
ccgttttcttaccaccct

SEQ ID NO: 146 - DNA, 19 bp, synthetic construct
  source 1..19: mol_type = other DNA
  modified_base 1..8: mod_base = OTHER; note = 2'OME or 2'MOE ribonucleotide
atggtttattccatggcca

SEQ ID NO: 147 - DNA, 19 bp, synthetic construct
  source 1..19: mol_type = other DNA
  modified_base 1..7: mod_base = OTHER; note = 2'OME or 2'MOE ribonucleotide
atggtttattccatggcca

SEQ ID NO: 148 - DNA, 19 bp, synthetic construct
  source 1..19: mol_type = other DNA
  misc_feature 1..8: note = 2'MOE ribonucleotide with phosphorothioate linkage
atggtttattccatggcca

SEQ ID NO: 149 - DNA, 19 bp, synthetic construct
  source 1..19: mol_type = other DNA
  misc_feature 1..7: note = 2'MOE ribonucleotide with phosphorothioate linkage
atggtttattccatggcca

SEQ ID NO: 150 - DNA, 21 bp, synthetic construct
  source 1..21: mol_type = other DNA
  modified_base 1..10: mod_base = OTHER; note = 2'OME or 2'MOE ribonucleotide
  misc_feature 1: note = DNA
  misc_feature 2..4: note = RNA
  misc_feature 5..21: note = DNA
  modified_base 2..4: mod_base = OTHER; note = uracil
ctttcagacacgggaacacgg

SEQ ID NO: 151 - DNA, 21 bp, synthetic construct
  source 1..21: mol_type = other DNA
  modified_base 1..9: mod_base = OTHER; note = 2'OME or 2'MOE ribonucleotide
  misc_feature 1: note = DNA
  misc_feature 2..4: note = RNA
  misc_feature 5..21: note = DNA
  modified_base 2..4: mod_base = OTHER; note = uracil
ctttcagacacgggaacacgg

SEQ ID NO: 152 - DNA, 21 bp, synthetic construct
  source 1..21: mol_type = other DNA
  misc_feature 1..10: note = 2'MOE ribonucleotide with phosphorothioate linkage
  misc_feature 1: note = DNA
  misc_feature 5..21: note = DNA
  misc_feature 2..4: note = RNA
  modified_base 2..4: mod_base = OTHER; note = uracil
ctttcagacacgggaacacgg

SEQ ID NO: 153 - DNA, 21 bp, synthetic construct
  source 1..21: mol_type = other DNA
  misc_feature 1..9: note = 2'MOE ribonucleotide with phosphorothioate linkage
  misc_feature 1: note = DNA
  misc_feature 2..4: note = RNA
  misc_feature 5..21: note = DNA
  modified_base 2..4: mod_base = OTHER; note = uracil
ctttcagacacgggaacacgg

SEQ ID NO: 154 - DNA, 20 bp, synthetic construct
  source 1..20: mol_type = other DNA
  modified_base 1..9: mod_base = OTHER; note = 2'OME or 2'MOE ribonucleotide
cttcacatttcgatccaaca

SEQ ID NO: 155 - DNA, 20 bp, synthetic construct
  source 1..20: mol_type = other DNA
  modified_base 1..8: mod_base = OTHER; note = 2'OME or 2'MOE ribonucleotide
cttcacatttcgatccaaca

SEQ ID NO: 156 - DNA, 20 bp, synthetic construct
  source 1..20: mol_type = other DNA
  misc_feature 1..9: note = 2'MOE ribonucleotide with phosphorothioate linkage
cttcacatttcgatccaaca

SEQ ID NO: 157 - DNA, 20 bp, synthetic construct
  source 1..20: mol_type = other DNA
  misc_feature 1..8: note = 2'MOE ribonucleotide with phosphorothioate linkage
cttcacatttcgatccaaca

SEQ ID NO: 158 - DNA, 18 bp, synthetic construct
  source 1..18: mol_type = other DNA
  modified_base 1..14: mod_base = OTHER; note = 2'OME or 2'MOE ribonucleotide
agttggagcaagattatc

SEQ ID NO: 159 - DNA, 18 bp, synthetic construct
  source 1..18: mol_type = other DNA
  misc_feature 1..14: note = 2'MOE ribonucleotide with phosphorothioate linkage
agttggagcaagattatc

SEQ ID NO: 160 - DNA, 22 bp, synthetic construct
  source 1..22: mol_type = other DNA
  modified_base 1..18: mod_base = OTHER; note = 2'OME or 2'MOE ribonucleotide
caatgccatcctggagttcctg

SEQ ID NO: 161 - DNA, 22 bp, synthetic construct
  source 1..22: mol_type = other DNA
  misc_feature 1..18: note = 2'MOE ribonucleotide with phosphorothioate linkage
caatgccatcctggagttcctg

SEQ ID NO: 162 - DNA, 25 bp, synthetic construct
  source 1..25: mol_type = other DNA
  modified_base 1..21: mod_base = OTHER; note = 2'OME or 2'MOE ribonucleotide
gttgcctccggttctgaaggtgttc

SEQ ID NO: 163 - DNA, 25 bp, synthetic construct
  source 1..25: mol_type = other DNA
  misc_feature 1..21: note = 2'MOE ribonucleotide with phosphorothioate linkage
gttgcctccggttctgaaggtgttc

SEQ ID NO: 164 - DNA, 30 bp, synthetic construct; identical to SEQ ID NO: 60
  source 1..30: mol_type = other DNA
  modified_base 1..26: mod_base = OTHER; note = 2'OME or 2'MOE ribonucleotide

SEQ ID NO: 165 - DNA, 30 bp, synthetic construct; identical to SEQ ID NO: 60
  source 1..30: mol_type = other DNA
  misc_feature 1..26: note = 2'MOE ribonucleotide with phosphorothioate linkage

SEQ ID NO: 166 - DNA, 20 bp, synthetic construct
  source 1..20: mol_type = other DNA
  modified_base 1..9: mod_base = OTHER; note = 2'OME or 2'MOE ribonucleotide
  modified_base 4: mod_base = OTHER; note = uracil
  modified_base 16: mod_base = OTHER; note = uracil
  misc_feature 1..3: note = DNA
  misc_feature 4: note = RNA
  misc_feature 5..15: note = DNA
  misc_feature 16: note = RNA
  misc_feature 17..20: note = DNA
ccatactttccactgtaacg

SEQ ID NO: 167 - DNA, 20 bp, synthetic construct
  source 1..20: mol_type = other DNA
  misc_feature 1..9: note = 2'MOE ribonucleotide with phosphorothioate linkage
ctcagtaacattgacaccac

SEQ ID NO: 168 - DNA, 20 bp, synthetic construct
  source 1..20: mol_type = other DNA
  misc_feature 1..8: note = 2'MOE ribonucleotide with phosphorothioate linkage
ctcagtaacattgacaccac

SEQ ID NO: 169 - DNA, 20 bp, synthetic construct
  source 1..20: mol_type = other DNA
  misc_feature 2: note = 2'MOE ribonucleotide with phosphorothioate linkage
  misc_feature 4: note = 2'MOE ribonucleotide with phosphorothioate linkage
  misc_feature 6: note = 2'MOE ribonucleotide with phosphorothioate linkage
  misc_feature 8..9: note = 2'MOE ribonucleotide with phosphorothioate linkage
  modified_base 1: mod_base = OTHER; note = 2'MOE phosphodiester
  modified_base 3: mod_base = OTHER; note = 2'MOE phosphodiester
  modified_base 5: mod_base = OTHER; note = 2'MOE phosphodiester
  modified_base 7: mod_base = OTHER; note = 2'MOE phosphodiester
ctcagtaacattgacaccac

SEQ ID NO: 170 - DNA, 20 bp, synthetic construct
  source 1..20: mol_type = other DNA
  misc_feature 2: note = 2'MOE ribonucleotide with phosphorothioate linkage
  misc_feature 4: note = 2'MOE ribonucleotide with phosphorothioate linkage
  misc_feature 8: note = 2'MOE ribonucleotide with phosphorothioate linkage
  misc_feature 6: note = 2'MOE ribonucleotide with phosphorothioate linkage
  modified_base 1: mod_base = OTHER; note = 2'MOE phosphodiester
  modified_base 3: mod_base = OTHER; note = 2'MOE phosphodiester
  modified_base 5: mod_base = OTHER; note = 2'MOE phosphodiester
  modified_base 7: mod_base = OTHER; note = 2'MOE phosphodiester
  modified_base 9: mod_base = OTHER; note = 2'MOE phosphodiester
  misc_feature 10..11: note = RNA
  misc_feature 1..9: note = DNA
  misc_feature 12..20: note = DNA
ctcagtaacattgacaccac

SEQ ID NO: 171 - DNA, 20 bp, synthetic construct
  source 1..20: mol_type = other DNA
  misc_feature 2: note = 2'MOE ribonucleotide with phosphorothioate linkage
  misc_feature 4: note = 2'MOE ribonucleotide with phosphorothioate linkage
  misc_feature 8..9: note = 2'MOE ribonucleotide with phosphorothioate linkage
  misc_feature 6: note = 2'MOE ribonucleotide with phosphorothioate linkage
  modified_base 1: mod_base = OTHER; note = 2'MOE phosphodiester
  modified_base 3: mod_base = OTHER; note = 2'MOE phosphodiester
  modified_base 5: mod_base = OTHER; note = 2'MOE phosphodiester
  modified_base 7: mod_base = OTHER; note = 2'MOE phosphodiester
  misc_feature 10..12: note = RNA
  misc_feature 1..9: note = DNA
  misc_feature 13..20: note = DNA
ctcagtaacattgacaccac

SEQ ID NO: 172 - DNA, 20 bp, synthetic construct
  source 1..20: mol_type = other DNA
  misc_feature 2: note = 2'MOE ribonucleotide with phosphorothioate linkage
  misc_feature 4: note = 2'MOE ribonucleotide with phosphorothioate linkage
  misc_feature 8..9: note = 2'MOE ribonucleotide with phosphorothioate linkage
  misc_feature 6: note = 2'MOE ribonucleotide with phosphorothioate linkage
  modified_base 1: mod_base = OTHER; note = 2'MOE phosphodiester
  modified_base 3: mod_base = OTHER; note = 2'MOE phosphodiester
  modified_base 5: mod_base = OTHER; note = 2'MOE phosphodiester
  modified_base 7: mod_base = OTHER; note = 2'MOE phosphodiester
  misc_feature 11..13: note = RNA
  misc_feature 1..10: note = DNA
  misc_feature 14..20: note = DNA
ctcagtaacattgacaccac

SEQ ID NO: 173 - DNA, 20 bp, synthetic construct
  source 1..20: mol_type = other DNA
  misc_feature 2: note = 2'MOE ribonucleotide with phosphorothioate linkage
  misc_feature 4: note = 2'MOE ribonucleotide with phosphorothioate linkage
  misc_feature 8..9: note = 2'MOE ribonucleotide with phosphorothioate linkage
  misc_feature 6: note = 2'MOE ribonucleotide with phosphorothioate linkage
  modified_base 1: mod_base = OTHER; note = 2'MOE phosphodiester
  modified_base 3: mod_base = OTHER; note = 2'MOE phosphodiester
  modified_base 5: mod_base = OTHER; note = 2'MOE phosphodiester
  modified_base 7: mod_base = OTHER; note = 2'MOE phosphodiester
  misc_feature 10..11: note = RNA
  misc_feature 1..9: note = DNA
  misc_feature 12..20: note = DNA
ctcagtaacattgacaccac

SEQ ID NO: 174 - DNA, 20 bp, synthetic construct
  source 1..20: mol_type = other DNA
  misc_feature 2: note = 2'MOE ribonucleotide with phosphorothioate linkage
  misc_feature 4: note = 2'MOE ribonucleotide with phosphorothioate linkage
  misc_feature 8..9: note = 2'MOE ribonucleotide with phosphorothioate linkage
  misc_feature 6: note = 2'MOE ribonucleotide with phosphorothioate linkage
  modified_base 1: mod_base = OTHER; note = 2'MOE phosphodiester
  modified_base 3: mod_base = OTHER; note = 2'MOE phosphodiester
  modified_base 5: mod_base = OTHER; note = 2'MOE phosphodiester
  modified_base 7: mod_base = OTHER; note = 2'MOE phosphodiester
  misc_feature 11..12: note = RNA
  misc_feature 1..10: note = DNA
  misc_feature 13..20: note = DNA
ctcagtaacattgacaccac

SEQ ID NO: 175 - DNA, 18 bp, synthetic construct
  source 1..18: mol_type = other DNA
  misc_feature 1..7: note = 2'MOE ribonucleotide with phosphorothioate linkage
ccgttttcttaccaccct

SEQ ID NO: 176 - DNA, 18 bp, synthetic construct
  source 1..18: mol_type = other DNA
  misc_feature 1..2: note = 2'MOE ribonucleotide with phosphorothioate linkage
  misc_feature 7: note = 2'MOE ribonucleotide with phosphorothioate linkage
  modified_base 3..6: mod_base = OTHER; note = 2'MOE phosphodiester
ccgttttcttaccaccct

SEQ ID NO: 177 - DNA, 20 bp, synthetic construct
  source 1..20: mol_type = other DNA
  misc_feature 3..8: note = 2'MOE ribonucleotide with phosphorothioate linkage
  modified_base 1..2: mod_base = OTHER; note = 2'MOE phosphodiester
caggatacatttctacagct

SEQ ID NO: 178 - DNA, 20 bp, synthetic construct
  source 1..20: mol_type = other DNA
  misc_feature 4..8: note = 2'MOE ribonucleotide with phosphorothioate linkage
  modified_base 1..3: mod_base = OTHER; note = 2'MOE phosphodiester
caggatacatttctacagct

SEQ ID NO: 179 - DNA, 20 bp, synthetic construct
  source 1..20: mol_type = other DNA
  misc_feature 5..8: note = 2'MOE ribonucleotide with phosphorothioate linkage
  modified_base 1..4: mod_base = OTHER; note = 2'MOE phosphodiester
caggatacatttctacagct

SEQ ID NO: 180 - DNA, 26 bp, synthetic construct
  source 1..26: mol_type = other DNA
  misc_feature 1: note = 2'MOE ribonucleotide with phosphorothioate linkage
  misc_feature 3: note = 2'MOE ribonucleotide with phosphorothioate linkage
  misc_feature 5: note = 2'MOE ribonucleotide with phosphorothioate linkage
  misc_feature 17: note = 2'MOE ribonucleotide with phosphorothioate linkage
  misc_feature 19..20: note = 2'MOE ribonucleotide with phosphorothioate linkage
  modified_base 2: mod_base = OTHER; note = 2'MOE phosphodiester
  modified_base 4: mod_base = OTHER; note = 2'MOE phosphodiester
  modified_base 16: mod_base = OTHER; note = 2'MOE phosphodiester
  modified_base 18: mod_base = OTHER; note = 2'MOE phosphodiester
  misc_feature 21..26: note = DNA phosphodiester
caggatacatttctacagctacagct

SEQ ID NO: 181 - DNA, 26 bp, synthetic construct
  source 1..26: mol_type = other DNA
  misc_feature 3..8: note = 2'MOE ribonucleotide with phosphorothioate linkage
  modified_base 1..2: mod_base = OTHER; note = 2'MOE phosphodiester
  misc_feature 21..26: note = DNA phosphodiester
caggatacatttctacagctacagct

SEQ ID NO: 182 - DNA, 26 bp, synthetic construct
  source 1..26: mol_type = other DNA
  misc_feature 4..8: note = 2'MOE ribonucleotide with phosphorothioate linkage
  modified_base 1..3: mod_base = OTHER; note = 2'MOE phosphodiester
  misc_feature 21..26: note = DNA phosphodiester
caggatacatttctacagctacagct

SEQ ID NO: 183 - DNA, 26 bp, synthetic construct
  source 1..26: mol_type = other DNA
  misc_feature 1..9: note = 2'MOE ribonucleotide with phosphorothioate linkage
  misc_feature 21..26: note = DNA phosphodiester
caggatacatttctacagctagcagg

SEQ ID NO: 184 - DNA, 26 bp, synthetic construct
  source 1..26: mol_type = other DNA
  misc_feature 4..9: note = 2'MOE ribonucleotide with phosphorothioate linkage
  modified_base 1..3: mod_base = OTHER; note = 2'MOE phosphodiester
  misc_feature 21..26: note = DNA phosphodiester
caggatacatttctacagctagcagg

SEQ ID NO: 185 - DNA, 20 bp, synthetic construct
  source 1..20: mol_type = other DNA
  misc_feature 1..5: note = 2'MOE ribonucleotide with phosphorothioate linkage
  misc_feature 16..20: note = 2'MOE ribonucleotide with phosphorothioate linkage
caggatacatttctacagct

SEQ ID NO: 186 - DNA, 27 bp, synthetic construct
  source 1..27: mol_type = other DNA
  misc_feature 1..5: note = 2'MOE ribonucleotide with phosphorothioate linkage
  misc_feature 17: note = 2'MOE ribonucleotide with phosphorothioate linkage
  misc_feature 19..20: note = 2'MOE ribonucleotide with phosphorothioate linkage
  modified_base 16: mod_base = OTHER; note = 2'MOE phosphodiester
  modified_base 18: mod_base = OTHER; note = 2'MOE phosphodiester
  misc_feature 21..27: note = DNA phosphodiester
caggatacatttctacagcttagcagg

SEQ ID NO: 187 - DNA, 27 bp, synthetic construct
  source 1..27: mol_type = other DNA
  misc_feature 1: note = 2'MOE ribonucleotide with phosphorothioate linkage
  misc_feature 3: note = 2'MOE ribonucleotide with phosphorothioate linkage
  misc_feature 5: note = 2'MOE ribonucleotide with phosphorothioate linkage
  misc_feature 17: note = 2'MOE ribonucleotide with phosphorothioate linkage
  misc_feature 19..20: note = 2'MOE ribonucleotide with phosphorothioate linkage
  modified_base 2: mod_base = OTHER; note = 2'MOE phosphodiester
  modified_base 4: mod_base = OTHER; note = 2'MOE phosphodiester
  modified_base 16: mod_base = OTHER; note = 2'MOE phosphodiester
  modified_base 18: mod_base = OTHER; note = 2'MOE phosphodiester
  misc_feature 21..27: note = DNA phosphodiester
caggatacatttctacagcttagcagg

SEQ ID NO: 188 - DNA, 20 bp, synthetic construct
  source 1..20: mol_type = other DNA
  misc_feature 1..9: note = 2'MOE ribonucleotide with phosphorothioate linkage
gcttcttgtccagctttatt

SEQ ID NO: 189 - DNA, 20 bp, synthetic construct
  source 1..20: mol_type = other DNA
  misc_feature 4..9: note = 2'MOE ribonucleotide with phosphorothioate linkage
  modified_base 1..3: mod_base = OTHER; note = 2'MOE phosphodiester
gcttcttgtccagctttatt

SEQ ID NO: 190 - DNA, 20 bp, synthetic construct
  source 1..20: mol_type = other DNA
  misc_feature 1..4: note = 2'MOE ribonucleotide with phosphorothioate linkage
  misc_feature 6..9: note = 2'MOE ribonucleotide with phosphorothioate linkage
gcttcttgtccagctttatt

SEQ ID NO: 191 - DNA, 25 bp, synthetic construct
  source 1..25: mol_type = other DNA
  misc_feature 1..9: note = 2'MOE ribonucleotide with phosphorothioate linkage
  misc_feature 21..25: note = DNA phosphodiester
gcttcttgtccagctttattgggag

SEQ ID NO: 192 - DNA, 25 bp, synthetic construct
  source 1..25: mol_type = other DNA
  misc_feature 4..9: note = 2'MOE ribonucleotide with phosphorothioate linkage
  modified_base 1..3: mod_base = OTHER; note = 2'MOE phosphodiester
  misc_feature 21..25: note = DNA phosphodiester
gcttcttgtccagctttattgggag